(19) United States
(12) Patent Application Publication
(10) Pub. No.: US 2025/0333528 A1
(43) Pub. Date: Oct. 30, 2025

(54) TRANSFERRIN RECEPTOR-BINDING DOMAINS AND PROTEINS COMPRISING THE SAME

(71) Applicant: Denali Therapeutics Inc., South San Francisco, CA (US)

(72) Inventors: Joshua I. Park, Castro Valley, CA (US); Hai L. Tran, San Francisco, CA (US)

(21) Appl. No.: 18/866,243

(22) PCT Filed: May 16, 2023
(86) PCT No.: PCT/US2023/022334

§ 371 Date: Nov. 15, 2024

Related U.S. Application Data
(60) Provisional application No. 63/342,313, filed on May 16, 2022.

(51) Int. Cl. C07K 16/28; C07K 14/47; C12N 9/24

(52) U.S. Cl. CPC C07K 16/2881; C07K 14/47; C12N 9/2402; C12Y 302/01045; C07K 2317/24; C07K 2317/52; C07K 2317/569; C07K 2317/92; C07K 2319/00

(57) ABSTRACT

Provided herein are transferrin receptor-binding domains, polypeptides comprising a transferrin receptor-binding domain, and antibodies and antibody fragments comprising a transferrin receptor-binding domain, and uses of the same.

### CROSS-REFERENCE TO RELATED APPLICATIONS

This application claims priority to U.S. Provisional Patent Application Ser. No. 63/342,313, filed May 16, 2022; the entire contents of which are herein incorporated by reference.

### SEQUENCE LISTING

The instant application contains a Sequence Listing which has been submitted electronically as an XML file name “47364-0022WO1.” The XML file, created on May 15, 2023, is 254,608 bytes in size. The material in the XML file is hereby incorporated by reference in its entirety.

### TECHNICAL FIELD

The present disclosure relates generally to polypeptides and protein constructs that include an antigen-binding domain that specifically bind to a transferrin receptor.

### BACKGROUND

The human brain is a highly vascular organ containing approximately 400 miles of blood vessels. These blood vessels are lined by closely linked endothelial cells to form the blood-brain barrier (BBB), which protects the brain from toxins by regulating the transfer of proteins, nutrients, and waste products. Direct delivery of therapeutics into the CNS has been attempted; however, this represents a highly invasive approach and distribution is often limited to areas close to the site of administration. Delivery of therapeutics across the BBB is less-invasive manner of treatment but presents its own challenges due to the relative impermeability of the BBB to large molecules. Accordingly, compositions that contain or result in expression of brain-penetrant therapeutics, and methods of treatment using the same, would represent a major advancement in effective treatments that target the CNS.

Transferrin receptor is the cognate receptor for transferrin that, among other functions, is needed for the import of iron into the cell and is regulated in response to intracellular iron concentration. Transferrin receptors are expressed on the endothelium of the blood-brain-barrier, and can mediate transcytosis of its ligands across the blood-brain barrier. Protein therapeutics containing a binding domain that specifically and reversibly interacts with transferrin receptor may achieve higher brain exposure than standard biotherapeutic molecules.

### SUMMARY

Provided herein are transferrin receptor-binding proteins including a camelid heavy chain variable domain. In some embodiments, the transferrin receptor-binding protein binds to human transferrin receptor with a K_{D} of from about 1 nM to about 6 μM (e.g., about 1 nM to about 1 mM, about 1 nM to about 500 nM, about 1 nM to about 100 nM, about 1 nM to about 50 nM, or about 1 nM to about 10 nM). In some embodiments, the transferrin receptor-binding protein binds to cynomolgus transferrin receptor with a K_{D} of from about 1 nM to about 2.5 mM (e.g., about 1 nM to about 1 mM, about 1 nM to about 500 nM, about 1 nM to about 100 nM, about 1 nM to about 50 nM, or about 1 nM to about 10 nM).

Also provided herein are transferrin receptor-binding domains that include: (a) a first sequence of G-X₁-T-X₂-X₃-X₄-X₅-X₆-M-X₇ (SEQ ID NO: 1), wherein X₁ is L or F, X₂ is S or F, X₃ is D or S or E, X₄ is T or S, X₅ is G or Y, X₆ is G or A, or X₇ is G or S; (b) a second sequence of A-I-X₈-X₉-X₁₀-X₁₁-X₁₂-X₁₃-T-X₁₄-Y-A-D-S-V-K-G (SEQ ID NO: 2), wherein X₈ is T or S, X₉ is W or S or G or F or Y, X₁₀ is S or N, X₁₁ is G or A, X₁₂ is R or S or G, X₁₃ is H or A, or X₁₄ is L or Y; and (c) a third sequence of A-X₁₅-D-X₁₆-X₁₇-X₁₈-X₁₉-X₂₀-X₂₁-X₂₂-X₂₃-X₂₄-X₂₅-X₂₆-D-Y (SEQ ID NO: 3), wherein X₁₅ is L or R, X₁₆ is V or A, X₁₇ is V or A, X₁₈ is G or A, X₁₉ is I or A, X₂₀ is G or A, X₂₁ is I or A, X₂₂ is E or A, X₂₃ V or A, X₂₄ is Q or A, X₂₅ is T or A, or X₂₆ is Y or F.

In some embodiments, the transferrin receptor-binding domain includes: (a) a first sequence selected from the group consisting of: GLTSDTGGMG (SEQ ID NO: 4), GFTFSTGGMG (SEQ ID NO: 12), GFTSDTGGMG (SEQ ID NO: 5), GLTFDTGGMG (SEQ ID NO: 6), GLTSSTGGMG (SEQ ID NO: 7), GLTSETGGMG (SEQ ID NO: 8), GLTSDTYGMG (SEQ ID NO: 9), GLTSDTGAMG (SEQ ID NO: 10), GLTSDTGGMS (SEQ ID NO: 11), GFTFSTGGMS (SEQ ID NO: 13), and GFTFSSGGMS (SEQ ID NO: 14); (b) a second sequence selected from the group consisting of: AITWSGRHTLYADSVKG (SEQ ID NO: 15), AISWSGRHTLYADSVKG (SEQ ID NO: 16), AITSSGRHTLYADSVKG (SEQ ID NO: 17), AITGSGRHTLYADSVKG (SEQ ID NO: 18), AITWNGRHTLYADSVKG (SEQ ID NO: 19), AITWSGSHTLYADSVKG (SEQ ID NO: 20), AITWSGGHTLYADSVKG (SEQ ID NO: 21), AITWSGRHTYYADSVKG (SEQ ID NO: 22), AITWSARHTLYADSVKG (SEQ ID NO: 23), AITWSGRATLYADSVKG (SEQ ID NO: 24), AITFSGRHTLYADSVKG (SEQ ID NO: 25), and AITYSGRHTLYADSVKG (SEQ ID NO: 26); and (c) a third sequence selected from the group consisting of: ALDVVGIGIEVQTYDY (SEQ ID NO: 27), ARDVVGIGIEVQTYDY (SEQ ID NO: 28), ALDAVGIGIEVQTYDY (SEQ ID NO: 29), ALDVAGIGIEVQTYDY (SEQ ID NO: 30), ALDVVAIGIEVQTYDY (SEQ ID NO: 31), ALDVVGAGIEVQTYDY (SEQ ID NO: 32), ALDVVGIAIEVQTYDY (SEQ ID NO: 33), ALDVVGIGAEVQTYDY (SEQ ID NO: 34), ALDVVGIGIAVQTYDY (SEQ ID NO: 35), ALDVVGIGIEAQTYDY (SEQ ID NO: 36), ALDVVGIGIEVATYDY (SEQ ID NO: 37), ALDVVGIGIEVQAYDY (SEQ ID NO: 38), and ALDVVGIGIEVQTEDY (SEQ ID NO: 39).

Also provided herein are transferrin receptor-binding domains that include: (a) a first sequence of G-X₁-T-L-D-X₂-X₃-A-I-X₄ (SEQ ID NO: 183), wherein X₁ is S or F, wherein X₂ is D, H, or Y, X₃ is Y or F, and X₄ is G or A; (b) a second sequence of CI-S-X₅-X₆-D-G-X₇-T-X₈-Y-X₉-D-X₁₀-V-K-G (SEQ ID NO: 184), wherein X₅ is S or R, X₆ is S or G, X₇ is I or R, X₈ is F or Y, X₉ is A or G, and X₁₀ is F or S; and (c) a third sequence of A-X₁₁-X₁₂-X₁₃-G-P-N-X₁₄-C-R-G-W-L-W-X₁₅-P-X₁₆-X₁₇-S-G-S(SEQ ID NO: 185), wherein X₁₁ is A or S, X₁₂ is K or H, X₁₃ is Y or D, X₁₄ is I or V, X₁₅ is V or E, X₁₆ is P or Q, and X₁₇ is V, I, or L (SEQ ID NO: 185).

In some embodiments, the transferrin receptor-binding domain includes: (a) a first sequence selected from the group consisting of: GSTLDDYAIG (SEQ ID NO: 186), GSTLDHYAIG (SEQ ID NO: 187), and GFTLDYFAIA (SEQ ID NO: 188); (b) a second sequence selected from the group consisting of: CISSSDGITFYGDFVKG (SEQ ID NO: 189), CISRSDGITYYADSVKG (SEQ ID NO: 190), and CISSGDGRTFYADSVKG (SEQ ID NO: 191); and (c) a third sequence selected from the group consisting of: AAKYGPNICRGWLWVPPVSGS (SEQ ID NO: 192), AAHYGPNVCRGWLWEPPISGS (SEQ ID NO: 193), and ASHDGPNVCRGWLWVPQLSGS (SEQ ID NO: 194).

Also provided herein are transferrin receptor-binding domains that includes: (a) a first sequence of G-X₁-T-X₂-X₃-X₄-X₅-X₆-M-X₇ (SEQ ID NO: 195), wherein X₁ is L or F, X₂ is S or F, X₃ is D or S or E, X₄ is T or S, X₅ is G or Y, X₆ is G or A, and X₇ is G or S; (b) a second sequence of A-I-X₈-X₉-X₁₀-X₁₁-X₁₂-X₁₃-T-X₁₄-Y-A-D-S-V-K-G (SEQ ID NO: 196), wherein X₈ is T or S, X₉ is W, S, G, F, or Y, X₁₀ is S or N, X₁₁ is G or A, X₁₂ is R, S, or G, X₁₃ is H or A, or X₁₄ is L or Y; and (c) a third sequence of A-X₁₅-D-X₁₆-X₁₇-X₁₈-X₁₉-X₂₀-X₂₁-X₂₂-X₂₃-X₂₄-X₂₅-X₂₆-D-Y (SEQ ID NO: 197), wherein X₁₅ is L or R, X₁₆ is V or A, X₁₇ is V or A, X₁₈ is G or A, X₁₉ is I or A, X₂₀ is G or A, X₂₁ is I or A, X₂₂ is E or A, X₂₃ V or A, X₂₄ is Q or A, X₂₅ is T or A, and X₂₆ is Y or F.

In some embodiments of any of the transferrin receptor-binding domains described herein, the transferrin receptor-binding domain is a VHH domain. In some embodiments, the VHH domain is humanized.

Also provided herein are polypeptides that include any of the transferrin receptor-binding domains described herein. In some embodiments, the polypeptide also includes an Fc polypeptide. In some embodiments of any of the polypeptides described herein, the polypeptide further includes a fusion partner. In some embodiments, the fusion partner is selected from the group consisting of: an enzyme, a hormone, a growth factor, a cytokine, a chemokine, a glycolipid, a lipid, a soluble protein, a nucleic acid, and an additional antigen-binding domain.

In some embodiments of any of the polypeptides described herein, the polypeptide includes a linker between the fusion partner and the transferrin receptor-binding domain. In some embodiments of any of the polypeptides described herein, the fusion partner is directly adjacent to the transferrin receptor-binding domain.

In some embodiments of any of the polypeptides described herein, the polypeptide is a single polypeptide. In some embodiments, the single polypeptide includes a sequence at least 80% identical to any one of SEQ ID NOs: 216-221. In some embodiments, the single polypeptide includes a sequence at least 90% identical to any one of SEQ ID NOs: 216-221. In some embodiments, the single polypeptide includes a sequence of any one of SEQ ID NOs: 216-221.

In some embodiments of any of the polypeptides described herein, the polypeptide is part of a protein complex.

Also provided herein are antibodies and antibody fragments including any of the transferrin receptor-binding domains described herein. In some embodiments, the antibody or antibody fragment includes a VHH domain. In some embodiments, the antibody is humanized.

In some embodiments, the antibody or antibody fragment further includes a fusion partner. In some embodiments, the fusion partner is selected from the group consisting of: an enzyme, a hormone, a growth factor, a cytokine, a chemokine, a glycolipid, a lipid, a soluble protein, a nucleic acid, and an additional antigen-binding domain.

In some embodiments, the fusion partner further includes a linker between the fusion partner and the transferrin receptor-binding domain. In some embodiments, the fusion partner is directly adjacent to the transferrin receptor-binding domain.

Also provided herein are compositions including (i) any of the transferrin receptor-binding domains described herein, any of the polypeptides described herein, any of the antibodies described herein, or any of the antibody fragments described herein, and (ii) a pharmaceutically acceptable carrier.

Also provided herein are kits including any of the compositions described herein.

Also provided herein are nucleic acids encoding any of the transferrin receptor-binding domains described herein, any of the polypeptides described herein, any of the antibodies described herein, or any of the antibody fragments described herein. Also provided herein are expression vectors including any of the nucleic acids described herein. Also provided herein are host cells transfected with any of the nucleic acids described herein or transduced with any of the expression vectors described herein.

Provided herein are methods of producing the transferrin receptor-binding domain, the polypeptide, the antibody, or the antibody fragment that include: (a) culturing any of the host cells described herein in a culture medium under conditions sufficient to allow for the production of the transferrin receptor-binding domain, the polypeptide, the antibody, or the antibody fragment; and (b) harvesting the transferrin receptor-binding domain, the polypeptide, the antibody, or the antibody fragment from the host cell or the culture medium.

In some embodiments of any of the methods described herein, the method further includes isolating the transferrin receptor-binding domain, the polypeptide, the antibody, or the antibody fragment.

In some embodiments of any of the methods described herein, the method further includes formulating the isolated transferrin receptor-binding domain, polypeptide, antibody, or antibody fragment.

Provided herein are transferrin receptor-binding domains that include a sequence that is at least 80% identical to any one of SEQ ID NOs: 40-88, 147, and 210-215. In some embodiments, the transferrin receptor-binding domain includes a sequence that is at least 90% identical to any one of SEQ ID NOs: 40-88, 147, and 210-215. In some embodiments, the transferrin receptor-binding domain includes a sequence that is any one of SEQ ID Nos: 40-88, 147, and 210-215.

In some embodiments of any of the transferrin receptor-binding domains described herein, the transferrin receptor-binding domain is a VHH domain. In some embodiments, the VHH domain is humanized.

Provided herein are polypeptides including any of the transferrin receptor-binding domains described herein. In some embodiments, the polypeptide also includes an Fc polypeptide. In some embodiments of any of the polypeptides described herein, the polypeptide further includes a fusion partner. In some embodiments, the fusion partner is selected from the group consisting of: an enzyme, a hormone, a growth factor, a cytokine, a chemokine, a glycolipid, a lipid, a soluble protein, a nucleic acid, and an additional antigen-binding domain.

In some embodiments of any of the polypeptides described herein, the polypeptide includes a linker between the fusion partner and the transferrin receptor-binding domain. In some embodiments of any of the polypeptides described herein, the fusion partner is directly adjacent to the transferrin receptor-binding domain.

In some embodiments of any of the polypeptides described herein, the polypeptide is a single polypeptide.

In some embodiments, the single polypeptide includes a sequence at least 80% identical to any one of SEQ ID Nos: 216-221. In some embodiments, the single polypeptide includes a sequence at least 90% identical to any one of SEQ ID Nos: 216-221. In some embodiments, the single polypeptide includes a sequence of any one of SEQ ID Nos: 216-221.

In some embodiments of any of the polypeptides described herein, the polypeptide is part of a protein complex.

Also provided herein are antibodies that include any of the transferrin receptor-binding domains described herein.

In some embodiments, the antibody is humanized.

In some embodiments of any of the antibodies described herein, the antibody further includes a fusion partner. In some embodiments, the fusion partner is selected from the group consisting of: an enzyme, a hormone, a growth factor, a cytokine, a chemokine, a glycolipid, a lipid, a soluble protein, a nucleic acid, and an additional antigen-binding domain.

In some embodiments of any of the antibodies described herein, the fusion partner further includes a linker between the fusion partner and the transferrin receptor-binding domain. In some embodiments of any of the antibodies described herein, the fusion partner is directly adjacent to the transferrin receptor-binding domain.

Also provided herein are antibody fragments that include any of the transferrin receptor-binding domains described herein. In some embodiments, the antibody fragment includes a VHH domain.

In some embodiments of any of the antibody fragments described herein, the antibody fragment is humanized.

In some embodiments of any of the antibody fragments described herein, the antibody fragment further includes a fusion partner. In some embodiments, the fusion partner is selected from the group consisting of: an enzyme, a hormone, a growth factor, a cytokine, a chemokine, a glycolipid, a lipid, a soluble protein, a nucleic acid, and an additional antigen-binding domain.

In some embodiments of any of the antibody fragments described herein, the fusion partner further includes a linker between the fusion partner and the transferrin receptor-binding domain. In some embodiments of any of the antibody fragments described herein, the fusion partner is directly adjacent to the transferrin receptor-binding domain.

Also provided herein are compositions including (i) any of the transferrin receptor-binding domains described herein, any of the polypeptides described herein, any of the antibodies described herein, or any of the antibody fragments described herein, and (ii) a pharmaceutically acceptable carrier.

Also provided herein are kits including any of the compositions described herein.

Also provided herein are nucleic acids encoding any of the transferrin receptor-binding domains described herein, any of the polypeptides described herein, any of the antibodies described herein, or any of the antibody fragments described herein.

Also provided herein are expression vectors including any of the nucleic acids described herein. Also provided herein are host cells transfected with any of the nucleic acids described herein or transduced with any of the expression vectors described herein.

Also provided herein are methods of producing any of the transferrin receptor-binding domains described herein, any of the polypeptides described herein, any of the antibodies described herein, or any of the antibody fragments described herein that include: (a) culturing any of the host cells described herein in a culture medium under conditions sufficient to allow for the production of the transferrin receptor-binding domain, the polypeptide, the antibody, or the antibody fragment; and (b) harvesting the transferrin receptor-binding domain, the polypeptide, the antibody, or the antibody fragment from the host cell or the culture medium.

In some embodiments of any of the methods described herein, the method further includes isolating the transferrin receptor-binding domain, the polypeptide, the antibody, or the antibody fragment.

In some embodiments of any of the methods described herein, the method further includes formulating the isolated transferrin receptor-binding domain, polypeptide, antibody, or antibody fragment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows a series of fluorescent images showing uptake of a polypeptide including an exemplary antigen-binding domain that binds specifically to transferrin receptor after culturing HEK293T cells (human transferrin receptor positive cells), CHO:cyTfR cells (cyno transferrin receptor-positive cells) and CHO cells (human and cyno transferrin receptor-negative cells) in the presence of 4.0 nM to 1000 nM of the polypeptide. Detection was performed using a fluorophore-conjugated secondary antibody that binds specifically to the polypeptide.

FIG. 2A shows a series of representative cell uptake/binding curves for exemplary transferrin receptor-binding domains generated as described herein. Cell uptake/binding curves were generated in HEK293T cells (human transferrin receptor positive cells).

FIG. 2B shows a series of representative cell uptake/binding curves for exemplary transferrin receptor-binding domains generated as described herein. Cell uptake/binding curves were generated in CHO:cyTfR cells (cyno transferrin receptor-positive cells).

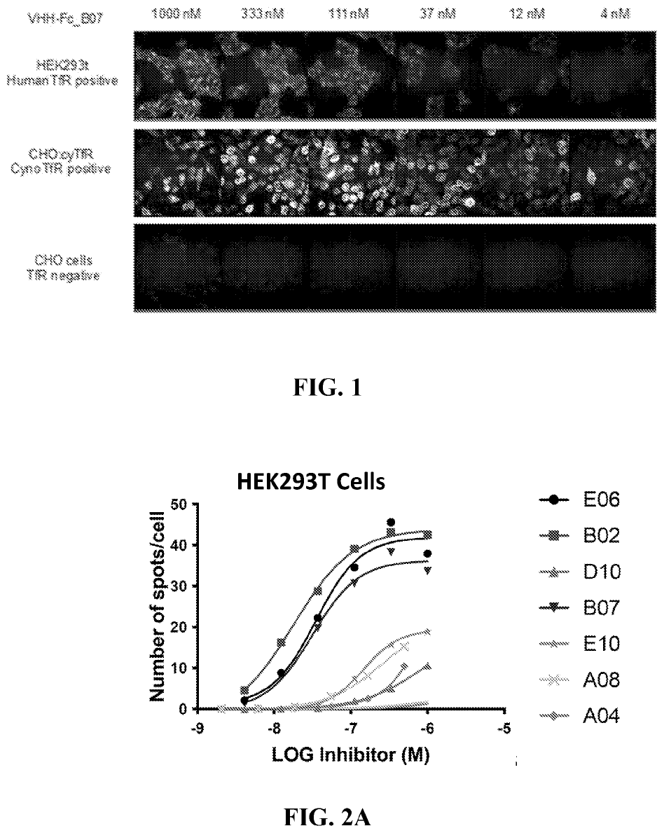

### DETAILED DESCRIPTION

Provided herein are transferrin receptor-binding proteins including a camelid heavy chain variable domain.

Also provided are polypeptides, antibodies, or antibody fragments that include any of the transferrin receptor-binding domains described herein. In some embodiments, the polypeptide is a single polypeptide. In other embodiments, the polypeptide is part of a protein complex.

Also provided herein are compositions that include any of the transferrin receptor-binding domains, polypeptides, antibodies, or antibody fragments described herein. Also provided herein are kits that include any of the compositions described herein. Also provided herein are nucleic acids that encode any of the transferrin receptor-binding domains, polypeptides, antibodies, or antibody fragments described herein. Also provided are host cells comprising any of the nucleic acids described herein. Also provided are methods of producing any of the transferrin receptor-binding domains, polypeptides, antibodies, and antibody fragments described herein that include culturing any of the host cells described herein.

In some embodiments of any of the antibodies, antibody fragments, or polypeptides described herein can include a total of about 50 to about 250 amino acids, about 100 to about 300 amino acids, about 150 to about 350 amino acids, about 200 to about 400 amino acids, about 600 to about 1,500 amino acids, about 650 to about 1,400 amino acids, about 700 to about 1,300 amino acids, about 750 to about 1,200 amino acids about 800 to about 1,100 amino acids, or about 900 to about 1,000 amino acids.

The term “a” and “an” refers to one or more (i.e., at least one) of the grammatical object of the article. By way of example, “a polypeptide” encompasses one or more polypeptides.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

Unless otherwise specified, a “nucleotide sequence encoding a protein” includes all nucleotide sequences that are degenerate versions of each other and thus encode the same amino acid sequence.

The term “exogenous” refers to any material introduced from or originating from outside a cell, a tissue or an organism that is not produced by or does not originate from the same cell, tissue, or organism in which it is being introduced.

The term “transduced,” “transfected,” or “transformed” refers to a process by which exogenous nucleic acid is introduced or transferred into a cell. A “transduced,” “transfected,” or “transformed” cell (e.g., a mammalian cell, a hepatocyte) is one that has been transduced, transfected or transformed with exogenous nucleic acid (e.g., an expression vector) that includes an exogenous nucleic acid encoding a polypeptide).

The term “subject” is intended to include any mammal. In some embodiments, the subject is cat, a dog, a goat, a human, a non-human primate, a rodent (e.g., a mouse or a rat), a pig, or a sheep. In some embodiments, the subject has or is at risk of developing a CNS disorder or disease. In some embodiments, the subject has previously been identified or diagnosed as having a CNS disorder or disease.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

The term “antigen-binding domain” is used to refer to one or more antibody variable domain(s) (e.g., formed from amino acids from a single polypeptide or formed from amino acids from two or more polypeptides (e.g., the same or different polypeptides)) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

The term “antibody” refers to a protein with an immunoglobulin fold that specifically binds to an antigen via its variable region or regions. The term “antibody” as used herein can refer to camelid (e.g., camel, llama, alpaca, dromedary) heavy-chain antibodies devoid of a light chain. These heavy-chain antibodies interact with an antigen through one single variable domain referred to as a VHH, VHH domain, or VHH antibody. The antigen binding site of a VHH domain resembles that of the heavy chain variable domain of conventional antibodies with differences in the framework and complementarity determining regions. A target antigen generally has numerous binding sites, also called epitopes, recognized by CDRs (complementarity determining regions) on multiple antibodies. Each antibody that specifically binds to a different epitope has a different structure. Thus, one antigen may have more than one corresponding antibody. An antibody can, e.g., include a full-length immunoglobulin molecule or an immunologically active portion of a full-length immunoglobulin molecule, i.e., a molecule that contains an antigen-binding site that immunospecifically binds an antigen of a target of interest or part thereof (e.g., transferrin receptor). The immunoglobulin disclosed herein can be of any subclass (e.g., IgG₁, IgG₂, IgG₃) of immunoglobulin molecule.

Antibodies bound to various types of molecules, such as polyethylene glycols (PEGs), may be used as modified antibodies. Methods for modifying antibodies are already established in the art.

The term “antibody fragments” refers to a portion of a full-length antibody or a polypeptide that includes a portion of a full-length antibody that retains antigen-binding activity via its variable region or regions. The term “antibody fragment” as used herein can refer to a portion of a full length camelid heavy chain antibody that retains antigen-binding activity via its variable region or regions. Non-limiting examples of antibody fragments that include a camelid heavy chain antibody that retains its antigen-binding activity include VHH domains, single-domain antibody, nanobodies, single-domain antibodies fused to Fc domains, and VHH domains fused to Fc domains (Bannas et al., Front. Immunol., 8: doi: 10.3389/fimmu.2017.01603 (2017)), Additional non-limiting examples of antibody fragments include “diabodies; linear antibodies; minibodies (Olafsen et al. (2004) Protein Eng. Design & Sel. 17(4):315-323), fragments produced by a Fab expression library, single-chain antibody molecules; and multispecific antibodies formed from antibody fragments.

The term “variable region” or “variable domain” refers to a domain in an antibody heavy chain or a domain in an antibody light chain that is encoded by a combination of a germline Variable (V) gene segment, Diversity (D) gene segment, or Joining (J) gene segment, and that confers on an antibody its specificity for binding to an antigen. As described herein, camelid heavy chain antibodies include a single variable heavy chain domain (e.g., a VHH domain) that is encoded by a combination of germline Variable (V) gene segments that confer the antibody with its specificity for binding to an antigen (e.g., a transferrin receptor).

The term “complementarity determining region” or “CDR” refers to one of the three hypervariable regions (or HVRs) that are known to confer (at least in part) antigen-binding specificity in each antibody light chain and each antibody heavy chain variable domain (e.g., VHH domain). The three CDRs in the antibody heavy chain and the antibody light chain interrupt four framework regions in the heavy chain variable domain and the light chain variable domain. The CDRs of each chain are typically referred to as CDR1, CDR2, and CDR3, numbered sequentially starting from the N-terminus, and are also typically identified by the chain in which the particular CDR is located. Thus, a VH CDR3 or CDR-H3 is the third CDR located in the heavy chain variable domain, whereas a VL CDR1 or CDR-L1 is the first CDR from the light chain variable domain. As used herein, VH CDR1 is used interchangeably with “a first sequence,” VH CDR2 is used interchangeably with “a second sequence,” and VH CDR3 is used interchangeably with “a third sequence.”

The “framework regions” or “FRs” of heavy immunoglobulin chains serve to position and align the CDRs in three-dimensional space. Framework sequences can be obtained from public DNA databases or published references that include germline antibody gene sequences.

The amino acid sequences of the CDRs and framework regions can be determined using various well-known definitions in the art, e.g., Kabat.

The term “protein complex” means a complex of two or more polypeptides (e.g., the same or different polypeptides) that associate through non-covalent bonds. For example, a protein complex can include at least one antigen-binding domain. Non-limiting examples and aspects of protein complexes are described herein. Additional examples and aspects of protein complexes are known in the art. In some embodiments, a protein complex includes one or more single polypeptides.

The term “treating” means a reduction in the number, frequency, severity, and/or duration of one or more (e.g., two, three, four, five, or six) symptoms of a disease or disorder in a subject (e.g., any of the subjects described herein), and/or results in a decrease in the rate of development and/or worsening of one or more symptoms of a disease or disorder in a subject over time.

The term “administer” refers to a method of delivering agents, compounds, or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. In one embodiment, the compositions described herein are administered intravenously.

The term “promoter” means a DNA sequence recognized by enzymes/proteins in a cell (e.g., a mammalian cell, a hepatocyte) required to initiate the transcription of an operably linked coding sequence (e.g., a nucleic acid encoding a polypeptide (e.g., any of the exemplary polypeptides described herein). A promoter typically refers, to e.g. a nucleotide sequence to which an RNA polymerase and/or any associated factor binds and at which transcription is initiated. The promoter can be constitutive, inducible, or tissue-specific (e.g., a liver-specific promoter).

The term “enhancer” refers to a nucleotide sequence that can increase the transcription of an operably linked nucleic acid (e.g., a nucleic acid encoding a polypeptide (e.g., any of the exemplary polypeptides described herein). An enhancer can increase the level of transcription by providing additional binding sites for transcription-associated proteins (e.g., transcription factors). Unlike promoters, enhancers can act at distances further away from the transcription start site (e.g., as compared to a promoter).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The term “affinity” refers to the strength of the sum of all non-covalent interactions between an antigen-binding site and its antigen. Unless otherwise indicated, “affinity” refers to intrinsic binding affinity, which reflects a 1:1 interaction between an antigen-binding domain and an antigen. Affinity can be measured, e.g., using surface plasmon resonance (SPR) technology (e.g., BIACORE®) or biolayer interferometry (e.g., FORTEBIO®). Additional methods for determining the affinity of an antigen-binding domain and its antigen are known in the art.

### Transferrin Receptor-Binding Domains and Polypeptides

Provided herein are transferrin receptor-binding proteins including a camelid heavy chain variable domain. In some embodiments, the protein binds to human transferrin receptor with a K_{D} of from about 0.1 nM to about 1 μM (e.g., about 0.1 nM to about 1 μM, about 0.5 nM to about 500 nM, about 1 nM to about 250 nM, about 1 nM to about 100 nM, about 1 nM to about 50 nM, or about 1 nM to about 10 nM). In some embodiments, the protein binds to cynomolgus transferrin receptor with a K_{D} of from about 1 nM to about 2.5 μM (e.g., about 1 nM to about 1 μM, about 1 nM to about 500 nM, about 1 nM to about 250 nM, about 1 nM to about 100 nM, about 1 nM to about 50 nM, or about 1 nM to about 10 nM).

Also provided herein are transferrin receptor-binding domains that include: (a) a first sequence of G-X₁-T-X₂-X₃-X₄-X₅-X₆-M-X₇ (SEQ ID NO: 1), wherein X₁ is L or F, X₂ is S or F, X₃ is D or S or E, X₄ is T or S, X₅ is G or Y, X₆ is G or A, or X₇ is G or S; (b) a second sequence of A-I-X₈-X₉-X₁₀-X₁₁-X₁₂-X₁₃-T-X₁₄-Y-A-D-S-V-K-G (SEQ ID NO: 2), wherein X₈ is T or S, X₉ is W or S or G or For Y, X₁₀ is S or N, X₁₁ is G or A, X₁₂ is R or S or G, X₁₃ is H or A, or X₁₄ is L or Y; and (c) a third sequence of A-X₁₅-D-X₁₆-X₁₇-X₁₈-X₁₉-X₂₀-X₂₁-X₂₂-X₂₃-X₂₄-X₂₅-X₂₆-D-Y (SEQ ID NO: 3), wherein X₁₅ is L or R, X₁₆ is V or A, X₁₇ is V or A, X₁₈ is G or A, X₁₉ is I or A, X₂₀ is G or A, X₂₁ is I or A, X₂₂ is E or A, X₂₃ V or A, X₂₄ is Q or A, X₂₅ is T or A, or X₂₆ is Y or F.

In some embodiments, the transferrin receptor-binding domain includes: (a) a first sequence selected from the group consisting of: GLTSDTGGMG (SEQ ID NO: 4), GFTSDTGGMG (SEQ ID NO: 5), GLTFDTGGMG (SEQ ID NO: 6), GLTSSTGGMG (SEQ ID NO: 7), GLTSETGGMG (SEQ ID NO: 8), GLTSDTYGMG (SEQ ID NO: 9), GLTSDTGAMG (SEQ ID NO: 10), GLTSDTGGMS (SEQ ID NO: 11), GFTFSTGGMG (SEQ ID NO: 12), GFTFSTGGMS (SEQ ID NO: 13), and GFTFSSGGMS (SEQ ID NO: 14); (b) a second sequence selected from the group consisting of: AITWSGRHTLYADSVKG (SEQ ID NO: 15), AISWSGRHTLYADSVKG (SEQ ID NO: 16), AITSSGRHTLYADSVKG (SEQ ID NO: 17), AITGSGRHTLYADSVKG (SEQ ID NO: 18), AITWNGRHTLYADSVKG (SEQ ID NO: 19), AITWSGSHTLYADSVKG (SEQ ID NO: 20), AITWSGGHTLYADSVKG (SEQ ID NO: 21), AITWSGRHTYYADSVKG (SEQ ID NO: 22), AITWSARHTLYADSVKG (SEQ ID NO: 23), AITWSGRATLYADSVKG (SEQ ID NO: 24), AITFSGRHTLYADSVKG (SEQ ID NO: 25), and AITYSGRHTLYADSVKG (SEQ ID NO: 26); and (c) a third sequence selected from the group consisting of: ALDVVGIGIEVQTYDY (SEQ ID NO: 27), ARDVVGIGIEVQTYDY (SEQ ID NO: 28), ALDAVGIGIEVQTYDY (SEQ ID NO: 29), ALDVAGIGIEVQTYDY (SEQ ID NO: 30), ALDVVAIGIEVQTYDY (SEQ ID NO: 31), ALDVVGAGIEVQTYDY (SEQ ID NO: 32), ALDVVGIAIEVQTYDY (SEQ ID NO: 33), ALDVVGIGAEVQTYDY (SEQ ID NO: 34), ALDVVGIGIAVQTYDY (SEQ ID NO: 35), ALDVVGIGIEAQTYDY (SEQ ID NO: 36), ALDVVGIGIEVATYDY (SEQ ID NO: 37), ALDVVGIGIEVQAYDY (SEQ ID NO: 38), and ALDVVGIGIEVQTEDY (SEQ ID NO: 39).

In some embodiments of any of the transferrin receptor-binding domains described herein, the transferrin receptor-binding domain includes: (a) GLTSDTGGMG (SEQ ID NO: 4), AITWSGRHTLYADSVKG (SEQ ID NO: 15), and ALDVVGIGIEVQTYDY (SEQ ID NO: 27); (b) GFTSDTGGMG (SEQ ID NO: 5), AITWSGRHTLYADSVKG (SEQ ID NO: 15), and ALDVVGIGIEVQTYDY (SEQ ID NO: 27); (c) GLTFDTGGMG (SEQ ID NO: 6), AITWSGRHTLYADSVKG (SEQ ID NO: 15), and ALDVVGIGIEVQTYDY (SEQ ID NO: 27); (d) GLTSSTGGMG (SEQ ID NO: 7), AITWSGRHTLYADSVKG (SEQ ID NO: 15), and ALDVVGIGIEVQTYDY (SEQ ID NO: 27); (e) GLTSETGGMG (SEQ ID NO: 8), AITWSGRHTLYADSVKG (SEQ ID NO: 15), and ALDVVGIGIEVQTYDY (SEQ ID NO: 27); (f) GLTSDTYGMG (SEQ ID NO: 9), AITWSGRHTLYADSVKG (SEQ ID NO: 15), and ALDVVGIGIEVQTYDY (SEQ ID NO: 27); (g) GLTSDTGAMG (SEQ ID NO: 10), AITWSGRHTLYADSVKG (SEQ ID NO: 15), and ALDVVGIGIEVQTYDY (SEQ ID NO: 27); (h) GLTSDTGGMS (SEQ ID NO: 11), AITWSGRHTLYADSVKG (SEQ ID NO: 15), and ALDVVGIGIEVQTYDY (SEQ ID NO: 27); (i) GLTSDTGGMG (SEQ ID NO: 4), AISWSGRHTLYADSVKG (SEQ ID NO: 16), and ALDVVGIGIEVQTYDY (SEQ ID NO: 27); (j) GLTSDTGGMG (SEQ ID NO: 4), AITSSGRHTLYADSVKG (SEQ ID NO: 17), and ALDVVGIGIEVQTYDY (SEQ ID NO: 27); (k) GLTSDTGGMG (SEQ ID NO: 4), AITGSGRHTLYADSVKG (SEQ ID NO: 18), and ALDVVGIGIEVQTYDY (SEQ ID NO: 27); (1) GLTSDTGGMG (SEQ ID NO: 4), AITWNGRHTLYADSVKG (SEQ ID NO: 19), and ALDVVGIGIEVQTYDY (SEQ ID NO: 27); (m) GLTSDTGGMG (SEQ ID NO: 4), AITWSGSHTLYADSVKG (SEQ ID NO: 20), and ALDVVGIGIEVQTYDY (SEQ ID NO: 27); (n) GLTSDTGGMG (SEQ ID NO: 4), AITWSGGHTLYADSVKG (SEQ ID NO: 21), and ALDVVGIGIEVQTYDY (SEQ ID NO: 27); (o) GLTSDTGGMG (SEQ ID NO: 4), AITWSGRHTYYADSVKG (SEQ ID NO: 22), and ALDVVGIGIEVQTYDY (SEQ ID NO: 27); (p) GLTSDTGGMG (SEQ ID NO: 4), AITWSGRHTLYADSVKG (SEQ ID NO: 15), and ARDVVGIGIEVQTYDY (SEQ ID NO: 28); (q) GLTSDTGGMG (SEQ ID NO: 4), AITWSGRHTLYADSVKG (SEQ ID NO: 15), and ALDAVGIGIEVQTYDY (SEQ ID NO: 29); (r) GLTSDTGGMG (SEQ ID NO: 4), AITWSGRHTLYADSVKG (SEQ ID NO: 15), and ALDVAGIGIEVQTYDY (SEQ ID NO: 30); (s) GLTSDTGGMG (SEQ ID NO: 4), AITWSGRHTLYADSVKG (SEQ ID NO: 15), and ALDVVAIGIEVQTYDY (SEQ ID NO: 31); (t) GLTSDTGGMG (SEQ ID NO: 4), AITWSGRHTLYADSVKG (SEQ ID NO: 15), and ALDVVGAGIEVQTYDY (SEQ ID NO: 32); (u) GLTSDTGGMG (SEQ ID NO: 4), AITWSGRHTLYADSVKG (SEQ ID NO: 15), and ALDVVGIAIEVQTYDY (SEQ ID NO: 33); (v) GLTSDTGGMG (SEQ ID NO: 4), AITWSGRHTLYADSVKG (SEQ ID NO: 15), and ALDVVGIGAEVQTYDY (SEQ ID NO: 34); (w) GLTSDTGGMG (SEQ ID NO: 4), AITWSGRHTLYADSVKG (SEQ ID NO: 15), and ALDVVGIGIAVQTYDY (SEQ ID NO: 35); (x) GLTSDTGGMG (SEQ ID NO: 4), AITWSGRHTLYADSVKG (SEQ ID NO: 15), and ALDVVGIGIEAQTYDY (SEQ ID NO: 36); (y) GLTSDTGGMG (SEQ ID NO: 4), AITWSGRHTLYADSVKG (SEQ ID NO: 15), and ALDVVGIGIEVATYDY (SEQ ID NO: 37); (z) GLTSDTGGMG (SEQ ID NO: 4), AITWSGRHTLYADSVKG (SEQ ID NO: 15), and ALDVVGIGIEVQAYDY (SEQ ID NO: 38); (aa) GLTSDTGGMG (SEQ ID NO: 4), AITWSGRHTLYADSVKG (SEQ ID NO: 15), and ALDVVGIGIEVQTEDY (SEQ ID NO: 39); (bb) GLTSDTGGMG (SEQ ID NO: 4), AITWSARHTLYADSVKG (SEQ ID NO: 23), and ALDVVGIGIEVQTYDY (SEQ ID NO: 27); (cc) GLTSDTGGMG (SEQ ID NO: 4), AITWSGRATLYADSVKG (SEQ ID NO: 24), and ALDVVGIGIEVQTYDY (SEQ ID NO: 27); (dd) GLTSDTGGMG (SEQ ID NO: 4), AITFSGRHTLYADSVKG (SEQ ID NO: 25), and ALDVVGIGIEVQTYDY (SEQ ID NO: 27); (ee) GFTFSTGGMG (SEQ ID NO: 12), AITWSGRHTLYADSVKG (SEQ ID NO: 15), and ALDVVGIGIEVQTYDY (SEQ ID NO: 27); (ff) GFTFSTGGMS (SEQ ID NO: 13), AITWSGRHTLYADSVKG (SEQ ID NO: 15), and ALDVVGIGIEVQTYDY (SEQ ID NO: 27); (gg) GFTFSSGGMS (SEQ ID NO: 14), AITWSGRHTLYADSVKG (SEQ ID NO: 15), and ALDVVGIGIEVQTYDY (SEQ ID NO: 27); (hh) GFTFSTGGMS (SEQ ID NO: 13), AITFSGRHTLYADSVKG (SEQ ID NO: 25), and ALDVVGIGIEVQTYDY (SEQ ID NO: 27); or (ii) GFTFSTGGMS (SEQ ID NO: 13), AITYSGRHTLYADSVKG (SEQ ID NO: 26), and ALDVVGIGIEVQTYDY (SEQ ID NO: 27).

Also provided herein are transferrin receptor-binding domains that include: (a) a first sequence of G-X₁-T-L-D-X₂-X₃-A-I-X₄ (SEQ ID NO: 183), wherein X₁ is S or F, wherein X₂ is D, H, or Y, X₃ is Y or F, and X₄ is G or A; (b) a second sequence of C-I-S-X₅-X₆-D-G-X₇-T-X₈-Y-X₉-D-X₁₀-V-K-G (SEQ ID NO: 184), wherein X₅ is S or R, X₆ is S or G, X₇ is I or R, X₈ is F or Y, X₉ is A or G, and X₁₀ is F or S; and (c) a third sequence of A-X₁₁-X₁₂-X₁₃-G-P-N-X₁₄-C-R-G-W-L-W-X₁₅-P-X₁₆-X₁₇-S-G-S(SEQ ID NO: 185), wherein X₁₁ is A or S, X₁₂ is K or H, X₁₃ is Y or D, X₁₄ is I or V, X₁₅ is V or E, X₁₆ is P or Q, and X₁₇ is V, I, or L (SEQ ID NO: 185).

In some embodiments, the transferrin receptor-binding domain includes: (a) a first sequence selected from the group consisting of: GSTLDDYAIG (SEQ ID NO: 186), GSTLDHYAIG (SEQ ID NO: 187), and GFTLDYFAIA (SEQ ID NO: 188); (b) a second sequence selected from the group consisting of: CISSSDGITFYGDFVKG (SEQ ID NO: 189), CISRSDGITYYADSVKG (SEQ ID NO: 190), and CISSGDGRTFYADSVKG (SEQ ID NO: 191); and (c) a third sequence selected from the group consisting of:

(SEQ ID NO: 192)
AAKYGPNICRGWLWVPPVSGS,
(SEQ ID NO: 193)
AAHYGPNVCRGWLWEPPISGS,
and
(SEQ ID NO: 194)
ASHDGPNVCRGWLWVPQLSGS.

In some embodiments of any of the transferring receptor-binding domains described herein, the transferrin receptor-binding domain includes: (a) GSTLDDYAIG (SEQ ID NO: 186), CISSSDGITFYGDFVKG (SEQ ID NO: 189), and AAKYGPNICRGWLWVPPVSGS (SEQ ID NO: 192); (b) GSTLDHYAIG (SEQ ID NO: 187), CISRSDGITYYADSVKG (SEQ ID NO: 190), and AAHYGPNVCRGWLWEPPISGS (SEQ ID NO: 193); or (c) GFTLDYFAIA (SEQ ID NO: 188), CISSGDGRTFYADSVKG (SEQ ID NO: 191), and ASHDGPNVCRGWLWVPQLSGS (SEQ ID NO: 194).

Also provided herein are transferrin receptor-binding domains that includes: (a) a first sequence of G-X₁-T-X₂-X₃-X₄-X₅-X₆-M-X₇ (SEQ ID NO: 195), wherein X₁ is L or F, X₂ is S or F, X₃ is D or S or E, X₄ is T or S, X₅ is G or Y, X₆ is G or A, and X₇ is G or S; (b) a second sequence of A-I-X₈-X₉-X₁₀-X₁₁-X₁₂-X₁₃-T-X₁₄-Y-A-D-S-V-K-G (SEQ ID NO: 196), wherein X₈ is T or S, X₉ is W, S, G, F, or Y, X₁₀ is S or N, X₁₁ is G or A, X₁₂ is R, S, or G, X₁₃ is H or A, or X₁₄ is L or Y; and (c) a third sequence of A-X₁₅-D-X₁₆-X₁₇-X₁₈-X₁₉-X₂₀-X₂₁-X₂₂-X₂₃-X₂₄-X₂₅-X₂₆-D-Y (SEQ ID NO: 197), wherein X₁₅ is L or R, X₁₆ is V or A, X₁₇ is V or A, X₁₈ is G or A, X₁₉ is I or A, X₂₀ is G or A, X₂₁ is I or A, X₂₂ is E or A, X₂₃ V or A, X₂₄ is Q or A, X₂₅ is T or A, and X₂₆ is Y or F. In some embodiments, the transferrin receptor-binding domain includes: GLTSDTGGMG (SEQ ID NO: 198), AITWSGRHTLYADSVKG (SEQ ID NO: 199), and ALDVVGIGIEVQTYDY (SEQ ID NO: 200).

Provided herein are transferrin receptor-binding domains that include: ESAFSLNAIG (SEQ ID NO: 201), GIGTDGITTYYADFVKD (SEQ ID NO: 202), and NAGSWRTVLSGTHVSRS (SEQ ID NO: 203).

Provided herein are transferrin receptor-binding domains that include:

(SEQ ID NO: 204)
GRDYNHFQRA,
(SEQ ID NO: 205)
RITWSGTITYNESVKG,
and
(SEQ ID NO: 206)
ALKTQPPLSQDAGDYTY.

Provided herein are transferrin receptor-binding domains that include:

(SEQ ID NO: 186)
GSTLDDYAIG,
(SEQ ID NO: 189)
CISSSDGITFYGDFVKG,
and
(SEQ ID NO: 192)
AAKYGPNICRGWLWVPPVSGS.

Provided herein are transferrin receptor-binding domains that include:

(SEQ ID NO: 210)
GRSLSTYVMG,
(SEQ ID NO: 211)
ARNGMSTYYTDSVKD,
and
(SEQ ID NO: 212)
AGDRSWSRLLRGEYEY.

Provided herein are transferrin receptor-binding domains that include:

(SEQ ID NO: 187)
GSTLDHYAIG,
(SEQ ID NO: 190)
CISRSDGITYYADSVKG,
and
(SEQ ID NO: 193)
AAHYGPNVCRGWLWEPPISGS.

Provided herein are transferrin receptor-binding domains that include: GFTLDYFAIA (SEQ ID NO: 188), CISSGDGRTFYADSVKG (SEQ ID NO: 191), and ASHDGPNVCRGWLWVPQLSGS (SEQ ID NO: 194).

Provided herein are transferrin receptor-binding domains that include:

(SEQ ID NO: 198)
GLTSDTGGMG,
(SEQ ID NO: 199)
AITWSGRHTLYADSVKG,
and
(SEQ ID NO: 200)
ALDVVGIGIEVQTYDY.

In some embodiments of any of the transferrin receptor-binding domains described herein, the transferrin receptor-binding domain is a humanized transferrin receptor-binding domain. In some embodiments of any of the antibodies including a transferrin receptor-binding domain described herein, the first, second, and third sequences are humanized.

In some embodiments of any of the transferrin receptor-binding domains described herein, the transferrin receptor-binding domain is a VHH domain. In some embodiments, the VHH domain is humanized.

Provided herein are transferrin receptor-binding domains that include a sequence that is at least 80% (e.g., at least 85%, at least 90%, at least 95%, at least 96%, at least 97%, at least 98%, at least 99%, or 100%) identical to any one of SEQ Id NOs: 40-88, 147, and 210-215.

Also provided herein are polypeptides that include any of the transferrin receptor-binding domains described herein. In some embodiments, the polypeptide also includes an Fc polypeptide.

In some embodiments, the polypeptide further comprises a fusion partner. In some embodiments, the fusion partner is selected from the group consisting of: an enzyme, a hormone, a growth factor, a cytokine, a chemokine, a glycolipid, a lipid, a soluble protein, a nucleic acid, and an additional antigen-binding domain.

In some embodiments, the polypeptide comprises a linker between the fusion partner and the transferrin receptor-binding domain. In some embodiments, the linker includes a total of about 1 amino acid to about 50 amino acids (e.g., about 1 amino acid to about 40 amino acids, about 1 amino acid to about 30 amino acids, about 1 amino acid to about 20 amino acids, about 1 amino acid to about 15 amino acids, about 1 amino acid to about 10 amino acids, or about 1 amino acid to about 5 amino acids). Non-limiting examples of linkers include: (G4S)n (SEQ ID NO: 153), wherein n is an integer between 1 and 10, including GGGGS (SEQ ID NO: 154), GGGGSGGGGS (SEQ ID NO: 155), GGGGSGGGGSGGGGS (SEQ ID NO: 156), or GGGGSGGGGSGGGGGGGGS (SEQ ID NO: 157).

In some embodiments, the fusion partner is directly adjacent to the transferrin receptor-binding domain.

In some embodiments, the polypeptide is a single polypeptide. In some embodiments, the single polypeptide comprises a transferrin receptor-binding domain comprising a sequence that is at least 80% (e.g., at least 85%, at least 90%, at least 95%, at least 96%, at least 98%, at least 99%, or 100%) identical to any one of SEQ ID Nos: 40-88, 147, and 210-215. In some embodiments, the single polypeptide comprises a sequence at least 80% (e.g., at least 85%, at least 90%, at least 95%, at least 96%, at least 98%, at least 99%, or 100%) identical to any one of SEQ ID Nos. 89-138 and 216-221.

In some embodiments of any of the single polypeptides described herein, the single polypeptide can be or include a nanobody, a nanobody-HSA, a camelid heavy chain antibody BiTE, a single variable domain, a monomer VHH domain, a polypeptide comprising two or more VHH domains, a polypeptide comprising a VHH domain and a second functional domain, a VHH-scFv, an IgG-VHH-scFv, or a Fc polypeptide-VHH-scFv.

In some embodiments of any of the single polypeptides described herein, the transferrin receptor-binding domain is fused to an Fc polypeptide.

In some embodiments of any of the single polypeptides described herein, the single polypeptide further includes a fusion partner (e.g., a heterologous fusion partner). In some embodiments, the fusion partner is an enzyme, a hormone, a growth factor, a cytokine, a chemokine, a glycolipid, a lipid, a soluble protein, a nucleic acid, or an additional antigen-binding domain (e.g., any of the exemplary antigen-binding domains described herein that bind to specifically to transferrin receptor, e.g., human transferrin receptor, or an antigen-binding domain that binds to a different antigen). In some embodiments, the fusion partner is glucosylceramidase beta (GBA). In some embodiments, the fusion partner is granulin precursor or progranulin (PGRN).

In some embodiments of any of the single polypeptides including GBA as a fusion partner, the single polypeptide includes a sequence that is at least 80% (e.g., at least 85%, at least 90%, at least 95%, at least 96%, at least 98%, at least 99%, or 100%) identical to SEQ ID NO: 179. In some embodiments of any of the single polypeptides including GBA as a fusion partner, the single polypeptide includes a sequence that is at least 80% (e.g., at least 85%, at least 90%, at least 95%, at least 96%, at least 98%, at least 99%, or 100%) identical to SEQ ID Nos: 139-144.

In some embodiments of any of the single polypeptides including PGRN as a fusion partner, the single polypeptide includes a sequence that is at least 80% (e.g., at least 85%, at least 90%, at least 95%, at least 96%, at least 98%, at least 99%, or 100%) identical to SEQ ID NO: 181. In some embodiments of any of the single polypeptides including PGRN as a fusion partner, the single polypeptide includes a sequence that is at least 80% (e.g., at least 85%, at least 90%, at least 95%, at least 96%, at least 98%, at least 99%, or 100%) identical to SEQ ID NO: 145, 146, or 148. In some embodiments of any of the single polypeptides including PGRN as a fusion partner, the single polypeptide includes a sequence that is at least 80% (e.g., at least 85%, at least 90%, at least 95%, at least 96%, at least 98%, at least 99%, or 100%) identical to SEQ ID NO: 147.

In some embodiments, the single polypeptide further includes a linker (e.g., any of the exemplary linkers described herein) between the fusion partner and the transferrin receptor-binding domain. In some embodiments, the fusion partner and the transferrin receptor-binding domain are directly adjacent to each other. In some embodiments, the single polypeptide comprises, from N-terminus to the C-terminus: a transferrin receptor-binding domain, a linker, and a fusion partner. In some embodiments, the single polypeptide comprises, from N-terminus to the C-terminus: a fusion partner, a linker, and a transferrin receptor-binding domain. In some embodiments, the linker includes a total of about 1 amino acid to about 50 amino acids (e.g., or any of the subranges of this range described herein). Non-limiting examples of linkers include: (G4S)n (SEQ ID NO: 153), wherein n is an integer between 1 and 10, including GGGGS (SEQ ID NO: 154), GGGGSGGGGS (SEQ ID NO: 155), GGGGSGGGGSGGGGS (SEQ ID NO: 156), or GGGGSGGGGSGGGGSGGGGS (SEQ ID NO: 157).

In some embodiments of any of the single polypeptides described herein, the single polypeptide does not include any other targeting domain.

In some embodiments, the polypeptide is part of a protein complex. In some embodiments, the protein complex includes at least a first polypeptide including any of the exemplary transferrin receptor-binding domains described herein.

In some embodiments, a protein complex can include at least a first polypeptide including a transferrin receptor-binding domain that includes a sequence that is at least 80% (e.g., at least 85%, at least 90%, at least 95%, at least 96%, at least 98%, at least 99%, or 100%) identical to any one of SEQ ID NOs: 40-88, 147, and 210-215. In some embodiments, the protein complex can include at least a first polypeptide including a sequence that is at least 80% (e.g., at least 85%, at least 90%, at least 95%, at least 96%, at least 98%, at least 99%, or 100%) identical to any one of SEQ ID NOs. 89-138 and 216-221.

In some embodiments of any of the protein complexes described herein, the first polypeptide and/or a second polypeptide further include a fusion partner (e.g., a heterologous fusion partner). In some embodiments, the fusion partner is an enzyme, a hormone, a growth factor, a cytokine, a chemokine, a glycolipid, a lipid, a soluble protein, a nucleic acid, or an additional antigen-binding domain (e.g., any of the exemplary antigen-binding domains described herein that bind to specifically to transferrin receptor, e.g., human transferrin receptor, or an antigen-binding domain that binds to a different antigen). In some embodiments, the fusion partner is glucosylceramidase beta (GBA). In some embodiments, the fusion partner is granulin precursor or progranulin (PGRN). In some embodiments of any of the protein complexes described herein, the protein complex includes a polypeptide containing a transferrin receptor-binding domain and a fusion partner, wherein the polypeptide is optionally fused to an Fc domain. In some embodiments, the polypeptide can include a linker (e.g., any of the exemplary linkers described herein) between the polypeptide and the Fc domain.

In some embodiments of any of the protein complexes having a first and/or a second polypeptide including GBA as a fusion partner, the first polypeptide and/or second polypeptide can include a sequence that is at least 80% (e.g., at least 85%, at least 90%, at least 95%, at least 96%, at least 98%, at least 99%, or 100%) identical to SEQ ID NO: 179. In some embodiments, the first polypeptide and/or second polypeptide includes a sequence that is at least 80% (e.g., at least 85%, at least 90%, at least 95%, at least 96%, at least 98%, at least 99%, or 100%) identical to SEQ ID NOs: 139-144.

In some embodiments of any of the protein complexes having a first and/or a second polypeptide including PGRN as a fusion partner, the first polypeptide and/or second polypeptide can include a sequence that is at least 80% (e.g., at least 85%, at least 90%, at least 95%, at least 96%, at least 98%, at least 99%, or 100%) identical to SEQ ID NO: 181. In some embodiments, the first polypeptide and/or second polypeptide can include a sequence that is at least 80% (e.g., at least 85%, at least 90%, at least 95%, at least 96%, at least 98%, at least 99%, or 100%) identical to SEQ ID NO: 145, 146, or 148.

In some embodiments of any of the protein complexes having a first and/or a second polypeptide including a fusion partner (e.g., a GBA polypeptide or a PGRN polypeptide), the first polypeptide and/or the second polypeptide can further include a linker (e.g., any of the exemplary linkers described herein) between the fusion partner and the transferrin receptor-binding domain. In some embodiments, the fusion partner and the transferrin receptor-binding domain are directly adjacent to each other. In some embodiments, the first polypeptide and/or the second polypeptide comprises, from N-terminus to the C-terminus: a transferrin receptor-binding domain, a linker, and a fusion partner (e.g., a GBA or a PGRN polypeptide). In some embodiments, the first polypeptide and/or the second polypeptide comprises, from N-terminus to the C-terminus direction: a fusion partner (e.g., a GBA or a PGRN polypeptide), a linker, and transferrin receptor-binding domain.

In some embodiments of any of the protein complexes having a first and/or a second polypeptide including a fusion partner (e.g., a GBA polypeptide or a PGRN polypeptide), the linker includes a total of about 1 amino acid to about 50 amino acids (e.g., or any of the subranges of this range described herein). Non-limiting examples of linkers include: (G4S)n (SEQ ID NO: 153), wherein n is an integer between 1 and 10, including GGGGS (SEQ ID NO: 154), GGGGSGGGGS (SEQ ID NO: 155), GGGGSGGGGSGGGGS (SEQ ID NO: 156), or GGGGSGGGGSGGGGSGGGGS (SEQ ID NO: 157).

In some embodiments of any of the protein complexes described herein, the protein complex includes: a camelid heavy chain antibody, a nanobody, a nanobody-HSA, a humanized camelid heavy chain antibody, a bispecific humanized camelid heavy chain antibody, a camelid heavy chain antibody BiTE, a single variable domain, a monomer VHH domain, a dimer of VHH domains, a bispecific dimer of VHH domains, a dimer containing one VHH domain and a second functional domain, VHH-Fc, VHH-IgG, IgG-VHH, a knobs-in-holes assembly, a VHH-scFv, a IgG-VHH-scFv, or a Fc-VHH-scFv.

In some embodiments, the first polypeptide and the second polypeptide form a human or a humanized antibody. In some embodiments of any of the protein complexes described herein, the complex does not include any other targeting domain.

In some embodiments of any of the protein complexes described herein, the protein complex includes at least a first polypeptide including a transferrin receptor-binding domain. Some embodiments of these protein complexes further include a second polypeptide that includes a fusion partner (e.g., any of the exemplary fusion partners described herein, e.g., a GBA or PGRN polypeptide). In such embodiments, the first polypeptide and/or second polypeptide can be fused or linked to an Fc domain.

In some embodiments of any of the protein complexes described herein, the protein complex can be human or humanized.

Also provided herein are nucleic acids that encode any of the transferrin receptor-binding domains or polypeptides described herein. Also provided herein are expression vectors that include any of the nucleic acids described herein that encode any of the transferrin receptor-binding domains or polypeptides described herein. In some examples, the expression vector described herein can further include a promoter (e.g., any of the exemplary promoters described herein or known in the art) and/or an enhancer (e.g., any of the exemplary enhancers described herein or known in the art), where the promoter and/or enhancer is/are operably linked to the nucleic acid sequence encoding any of the transferrin receptor-binding domains or polypeptides described herein.

Also provided herein are nucleic acids encoding each of the polypeptides in any of the protein complexes described herein. Also provided herein are pairs or sets of nucleic acids that together encode each of the polypeptides in any of the protein complexes described herein.

Also provided herein are expression vectors that include any of the nucleic acids described herein. Also provided are pairs or sets of expression vectors that together encode each of the polypeptides in any of the protein complexes described herein. In some examples, the expression vector(s) described herein can further include a promoter (e.g., any of the exemplary promoters described herein or known in the art) and/or an enhancer (e.g., any of the exemplary enhancers described herein or known in the art), where the promoter and/or enhancer is/are operably linked to the nucleic acid sequence encoding any of the polypeptides described herein. Additional aspects and examples of expression vectors are described herein.

### Antibodies and Antibody Fragments

Provided herein are antibodies or antibody fragments that include any of the transferrin receptor-binding domains described herein. In some embodiments, the transferrin receptor-binding domain is a humanized transferrin receptor-binding domain. In some embodiments, the first, second, and third sequences of the transferrin receptor-binding domain are humanized.

In some embodiments, the antibody or antibody fragment can include a transferrin-receptor binding domain having a sequence that is at least 80% (e.g., at least 85%, at least 90%, at least 95%, at least 96%, at least 98%, at least 99%, or 100%) identical to any one of SEQ ID NOs. 40-88, 147, and 210-215.

In some embodiments, the antibody or antibody fragment includes a VHH domain. In some embodiments, the antibody or antibody fragment can include one or more additional single variable domains (e.g., VHH domain) that include an antigen-binding domain that binds specifically to a transferrin receptor (a transferrin receptor-binding domain, e.g., any of the exemplary transferrin receptor-binding domains described herein). In some embodiments, the antibody or antibody fragment includes one or more additional single variable domains that bind to a different antigen. In some embodiments, the antibody or antibody fragment does not include any other targeting domain.

In some embodiments, the antibody can be or can include an antibody, camelid heavy chain antibody, a nanobody, a nanobody-HSA, a humanized camelid heavy chain antibody, a bispecific humanized camelid heavy chain antibody, a camelid heavy chain antibody BiTE, a single variable domain, a monomer VHH domain, a dimer of VHH domains, a bispecific dimer of VHH domains, a dimer containing one VHH domain and a second functional domain, VHH-Fc, VHH-IgG, IgG-VHH, a knobs-in-holes assembly, a VHH-scFv, a IgG-VHH-scFv, or a Fc-VHH-scFv. In some embodiments, the antibody fragment can be or include: a camelid heavy chain antibody, a nanobody, a nanobody-HSA, a humanized camelid heavy chain antibody, a bispecific humanized camelid heavy chain antibody, a camelid heavy chain antibody BiTE, a single variable domain, a monomer VHH domain, a dimer of VHH domains, a bispecific dimer of VHH domains, a dimer containing one VHH domain and a second functional domain, VHH-Fc, VHH-IgG, IgG-VHH, a knobs-in-holes assembly, a VHH-scFv, a IgG-VHH-scFv, or a Fc-VHH-scFv.

In some embodiments, the antibody or antibody fragment includes an antigen-binding domain that binds specifically to a transferrin receptor (e.g., a transferrin receptor-binding domain), where the antigen-binding domain is fused to an Fc domain. In some embodiments, the antibody or antibody fragment can include a sequence that is at least 80% (e.g., at least 85%, at least 90%, at least 95%, at least 96%, at least 98%, at least 99%, or 100%) identical to any one of SEQ ID NOs: 89-138 or 216-221.

In some embodiments, the antibody or antibody fragment can be a human or a humanized antibody or antibody fragment.

In some embodiments, the antibody or antibody fragment further includes a fusion partner (e.g., a heterologous fusion partner) (e.g., any of the exemplary fusion partners described herein). In some embodiments, the fusion partner is an enzyme, a hormone, a growth factor, a cytokine, a chemokine, a glycolipid, a lipid, a soluble protein, a nucleic acid, or an additional antigen-binding domain (e.g., any of the exemplary antigen-binding domains described herein that bind to specifically to transferrin receptor, e.g., human transferrin receptor, or an antigen-binding domain that binds to a different antigen). In some embodiments, the fusion partner is a glucosylceramidase beta (GBA) polypeptide. In some embodiments, the fusion partner is a granulin precursor or progranulin (PGRN) polypeptide.

In some embodiments, the antibody or antibody fragment includes a GBA polypeptide as a fusion partner, wherein the GBA polypeptide has a sequence that is at least 80% (e.g., at least 85%, at least 90%, at least 95%, at least 96%, at least 98%, at least 99%, or 100%) identical to SEQ ID NO: 179. In some embodiments, the antibody or antibody fragment includes a sequence that is at least 80% (e.g., at least 85%, at least 90%, at least 95%, at least 96%, at least 98%, at least 99%, or 100%) identical to any one of SEQ ID NOs: 139-144.

In some embodiments, the antibody or antibody fragment includes a PGRN polypeptide as a fusion partner, wherein the PGRN polypeptide has a sequence that is at least 80% (e.g., at least 85%, at least 90%, at least 95%, at least 96%, at least 98%, at least 99%, or 100%) identical to SEQ ID NO: 181. In some embodiments, the antibody or antibody fragment includes a sequence that is at least 80% (e.g., at least 85%, at least 90%, at least 95%, at least 96%, at least 98%, at least 99%, or 100%) identical to any one of SEQ ID NOs: 145, 146, or 148.

In some embodiments, the antibody or antibody fragment further includes a linker (e.g., any of the exemplary linkers described herein) between the fusion partner and the transferrin receptor-binding domain. In some embodiments, the fusion partner and the transferrin receptor-binding domain are directly adjacent to each other. In some embodiments, the antibody or antibody fragment comprises, from N-terminus to the C-terminus: a transferrin receptor-binding domain, a linker, and a fusion partner (e.g., a GBA or a PGRN polypeptide). In some embodiments, the antibody or antibody fragment comprises, from N-terminus to the C-terminus direction: a fusion partner (e.g., a GBA or a PGRN polypeptide), a linker, and transferrin receptor-binding domain.

In some embodiments, the linker includes a total of about 1 amino acid to about 50 amino acids (e.g., or any of the subranges of this range described herein). Non-limiting examples of linkers include: (G4S)n (SEQ ID NO: 153), wherein n is an integer between 1 and 10, including GGGGS (SEQ ID NO: 154), GGGGSGGGGS (SEQ ID NO: 155), GGGGSGGGGSGGGGS (SEQ ID NO: 156), or GGGGSGGGGSGGGGSGGGGS (SEQ ID NO: 157).

Also provided herein are nucleic acids that encode any of the antibodies or antibody fragments described herein. Also provided herein are expression vectors that include any of the nucleic acids described herein that encode any of the antibodies or antibody fragments described herein. In some examples, the expression vector described herein can further include a promoter (e.g., any of the exemplary promoters described herein or known in the art) and/or an enhancer (e.g., any of the exemplary enhancers described herein or known in the art), where the promoter and/or enhancer is/are operably linked to the nucleic acid sequence encoding any of the antibodies or antibody fragments described herein. Additional aspects and examples of expression vectors are described herein.

### Additional Exemplary Aspects

The transferrin receptor-binding domains, single polypeptides, protein complexes, antibodies, and antibody fragments described herein include at least one transferrin receptor-binding domain. Exemplary human wildtype transferrin receptor proteins can include a sequence of SEQ ID NO: 149, 150, 151, or 152.

In some embodiments, the transferrin receptor-binding domain binds to a transferrin receptor (e.g., a human transferrin receptor) with a dissociation constant (K_{D}) of less than about 1×10⁻⁵ M, less than about 1×10⁻⁶ M, or less than about 1×10⁻⁷ M (e.g., less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M). In some embodiments, the affinity of the transferrin receptor-binding domain is determined by surface plasmon resonance (e.g., performed in phosphate buffered saline).

In some embodiments wherein the antibody, antibody fragment, single polypeptide, or protein complex includes a transferrin receptor-binding domain fused to an Fc polypeptide or Fc domain, the antibody, antibody fragment, or single polypeptide can include a linker (e.g., any of the exemplary linkers described herein) between the transferrin receptor-binding domain and the Fc region. In some embodiments, the linker includes a total of about 1 amino acid to about 50 amino acids (e.g., or any of the subranges of this range described herein). Non-limiting examples of linkers include: (G4S)n (SEQ ID NO: 153), wherein n is an integer between 1 and 10, including GGGGS (SEQ ID NO: 154), GGGGSGGGGS (SEQ ID NO: 155), GGGGSGGGGSGGGGS (SEQ ID NO: 156), or GGGGSGGGGSGGGGSGGGGS (SEQ ID NO: 157). In some embodiments, the transferrin receptor-binding domain and the Fc domain are directly adjacent.

Non-limiting examples of sequences that bind specifically to human transferrin receptor include SEQ Id NOs: 40-88, 147, and 89-138.

In some embodiments, any of the transferrin receptor-binding domains, single polypeptides, protein complexes, antibodies, and antibody fragments described herein can include a modified Fc region (e.g., a modified CH2 and/or CHD3 domain of a human Fc region (e.g., a human IgG1 Fc region, a human IgG2 Fc region, a human IgG3 Fc region, or a human IgG4 Fc region)).

### Expression Vectors

Non-limiting examples of expression vectors include plasmids and viral vectors. In some embodiments, the expression vectors are plasmids, adeno-associated viral (AAV) vectors, lentiviral vectors, sindbis virus vectors, alphavirus-based vectors, or adenoviral vectors. AAV vectors are generally described in, e.g., Asokan et al., Mol. Ther. 20:699-708, 2012, and B. J. Carter, in “Handbook of Parvoviruses”, Ed., P. Tijsser, CRC Press, pp. 155-168, 1990. Adenoviral vectors are generally described in, e.g., Wold and Toth, Curr. Gene Ther. 13 (6): 421-433, 2013; Baron et al., Curr. Opin. Virol. 29:1-7, 2018; and Barry, Expert Rev. Vaccines 17(2):163-173, 2018. Lentiviral vectors are generally described in, e.g., Milone and O'Doherty, Leukemia 32(7):1529-1541, 2018, Zheng et al., Anat. Rec. 301(5): 825-836, 2018; and Cai et al., Curr. Gene Ther. 16(3): 194-206, 2016.

Some embodiments of any of the expression vectors described herein, can include a promoter and/or enhancer operably linked to a nucleic acid encoding a transferrin receptor-binding domain (e.g., any of the exemplary transferrin receptor-binding domains described herein), a polypeptide (e.g., any of the exemplary polypeptides described herein), an antibody (e.g., any of the exemplary antibodies described herein), an antibody fragment (e.g., any of the exemplary antibody fragments described herein), or a protein complex (e.g., any of the exemplary protein complexes described herein).

In some embodiments, the expression vector can be an AAV vector. For example, an AAV vector can be selected from the group of: an AAV2 vector, an AAV5 vector, and an AAV8 vector, an AAV1 vector, an AAV7 vector, an AAV9 vector, an AAV3 vector, an AAV6 vector, an AAV10 vector, and an AAV11 vector. In any of the exemplary AAV vectors described herein, the AAV vector can include a AAV8 capsid protein, an AAV5 capsid protein, an AAV-LK03 capsid protein, an AAV-NK59 capsid protein, an AAV1 capsid protein, an AAV2 capsid protein, an AAV3 capsid protein, an AAV4 capsid protein, an AAV6 capsid protein, an AAV7 capsid protein, and an AAV9 capsid protein.

In some embodiments, the nucleic acid further includes a promoter and/or enhancer operably linked to the sequence encoding any of the transferrin receptor-binding domains, antibodies, antibody fragments, single polypeptide, or protein complexes described herein. In some embodiments, the promoter is constitutive. In some embodiments, the promoter is inducible. In some embodiments, the promoter is a tissue-specific promoter. Exemplary promoters that are constitutive, inducible, and/or tissue-specific are known in the art. Non-limiting examples of promoters include a CMV promoter, a CAG promoter, human alpha-1-antitrypsin (hAAT) promoter (e.g. SEQ ID NO: 167), an HLP promoter (e.g. SEQ ID NO: 166), an HCB promoter (e.g. SEQ ID NO:168), and a transthyretin promoter (e.g. SEQ ID NO: 169). Non-limiting examples of enhancers are an apolipoprotein E (ApoE) enhancer (e.g. SEQ ID NO:174) and serpin enhancer (e.g. SEQ ID NO:175). Other promoters and enhancers are described herein.

In some embodiments, the nucleic acid includes a promoter operably linked to the nucleic acid sequence encoding a fusion partner (e.g., any of the exemplary fusion partners described herein, e.g., a heterologous fusion partner)). In some embodiments, the nucleic acid includes an enhancer operably linked to the nucleic acid sequence encoding the fusion partner (e.g., any of the exemplary fusion partners described herein). In some embodiments, the nucleic acid includes an enhancer and a promoter operably linked to the nucleic acid sequence encoding the fusion partner (e.g., any of the exemplary fusion partners described herein).

In some embodiments, the nucleic acid that includes a promoter and/or a enhancer operably linked to a nucleic acid encoding a first polypeptide, a second polypeptide, or both (e.g., any of the exemplary first polypeptides described herein, any of the exemplary second polypeptides described herein) is flanked at its 5′ and 3′ end by viral inverted terminal repeat (ITR) sequences (e.g., any of the exemplary ITR sequences described herein or known in the art). In some embodiments, the nucleic acid includes a promoter and/or an enhancer operably linked to the nucleic acid sequence encoding a first polypeptide, a second polypeptide, or both (e.g., any of the exemplary first polypeptides described herein, any of the exemplary second polypeptides described herein) flanked at its 5′ and 3′ end by viral ITR sequences (e.g., any of the viral ITR sequences described herein or known in the art).

Exemplary ITR sequences are described in B. J. Carter, in “Handbook of Parvoviruses”, ed., P. Tijsser, CRC Press, pp. 155-168, 1990, and U.S. Pat. No. 9,150,882 (incorporated herein by reference). Typical AAV ITR sequences are about 80 to about 200 nucleotides in length (e.g., about 80 to about 200 nucleotides, about 80 to about 180 nucleotides, about 80 to about 160 nucleotides, about 80 to about 140 nucleotides, about 80 to about 120 nucleotides, about 80 to about 100 nucleotides, about 100 to about 200 nucleotides, about 100 to about 180 nucleotides, about 100 to about 160 nucleotides, about 100 to about 150 nucleotides, about 100 to about 140 nucleotides, about 100 to about 120 nucleotides, about 120 to about 200 nucleotides, about 120 to about 180 nucleotides, about 120 to about 160 nucleotides, about 120 to about 150 nucleotides, about 120 to about 140 nucleotides, about 140 to about 200 nucleotides, about 140 to about 180 nucleotides, about 140 to about 160 nucleotides, about 140 to about 150 nucleotides, about 160 to about 200 nucleotides, about 160 to about 180 nucleotides, or about 180 to about 200 nucleotides).

The ability to modify ITR sequences are within the skill of the art. See, e.g., texts such as Sambrook et al., “Molecular Cloning: A Laboratory Manual,” 2d Ed., Cold Spring Harbor Laboratory, New York, 1989; and Fischer et al., J. Virol. 70:520-532, 1996). Additional examples of ITR sequences may obtained from any known AAV (an AAV ITR). In some examples, the AAV ITR sequences can be AAV2 ITR sequences, or functional variants thereof. In some embodiments of any of the AAV vectors described herein, the ITR sequences are self-complementary ITR (scITR) sequences.

In some embodiments, the nucleic acid encoding the polypeptide, antibody, antibody fragment, or protein complex includes a single antigen-binding domain that specifically binds to the transferrin receptor. For example, in some embodiments, the nucleic acid sequence encoding the polypeptide, antibody fragment, antibody, or protein complex encodes a single binding domain that specifically binds to the transferrin receptor and does not encode another binding domain.

In some embodiments, the expression vector does not include any other targeting domain or amino acid sequence. In some embodiments of any of the expression vectors described herein, the expression vector includes only the first sequence, a linker, and the second sequence.

In some embodiments, the expression vectors described herein can include a nucleic acid encoding a protein tag sequence (e.g., a human influenza hemagglutinin (HA) tag, a c-Myc tag, or any other protein tag sequence known in the art). The presence of the HA-tag in a cell or in the CNS of a subject (e.g., a hepatocyte) can be detected by protein binding assays (e.g., Western blot, immunohistochemistry, radioimmunoassay (RIA)), or chemiluminescence).

In some embodiments, the expression vectors described herein include one or more (e.g., two, three, four, five, or six) of a promoter (e.g., any of the promoters described herein or known in the art), an enhancer (e.g., any of the enhancers described herein or known in the art), a Kozak sequence (e.g., any of the Kozak sequences described herein or known in the art), an RNA splicing sequence, a polyadenylation (poly(A)) signal sequence (e.g., any of the poly(A) signals described herein), and an internal ribosome entry site (IRES) sequence (e.g., any of the IRES sequences described herein or known in the art).

### Methods of Producing a Transferrin Receptor-Binding Domain, a Polypeptide, an Antibody, or Antibody Fragment

Also provided herein are methods of producing any of the transferrin receptor-binding domains, polypeptides, antibodies, or antibody fragments described herein that include: (a) culturing a cell (e.g., any of the cells described herein) including any of the nucleic acids encoding any of the transferrin receptor-binding domains, polypeptides, antibodies, or antibody fragments described herein, or any of the expression vectors described herein that include a nucleic acid encoding any of the transferrin receptor-binding domains, polypeptides, antibodies, or antibody fragments described herein, in a culture medium under conditions sufficient to allow for the production of the transferrin receptor-binding domain, polypeptide, antibody, or antibody fragment; and (b) harvesting the transferrin receptor-binding domain, polypeptide, antibody, or antibody fragment from the host cell or the culture medium. In some embodiments of any of the methods described herein, the method further includes isolating the transferrin receptor-binding domain, polypeptide, antibody, or antibody fragment (e.g., through performance of one or more column chromatography steps, ultrafiltration/diafiltration, and/or viral inactivation). In some embodiments of any of the methods described herein, the method further includes formulating the isolated transferrin receptor-binding domain, polypeptide, antibody, or antibody fragment into a composition (e.g., a pharmaceutical composition).

Any of the transferrin receptor-binding domains, polypeptides, antibodies, or antibody fragments described herein can be produced by any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. For example, a host cell can be a CHO cell or a HEK cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or expression vectors (e.g., any of the exemplary expression vectors described herein (e.g., an AAV vector)) into cells (e.g., mammalian cells) are known in the art. Non-limiting examples of methods that can be used to introduce a nucleic acid (e.g., any of the exemplary nucleic acids described herein) and/or an expression vector (e.g., any of the exemplary expression vectors described herein (e.g., an AAV vector)) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

Also provided herein are methods that further include isolation of the transferrin receptor-binding domain, polypeptide, antibody, or antibody fragment from cell culture medium or from a cell (e.g., a mammalian cell) using techniques well-known in the art (e.g., ion exchange chromatography (anionic or cation), metal-affinity chromatography, ligand-affinity chromatography, size exclusion chromatography, hydrophobic interaction chromatography, and precipitation (e.g., ammonium sulfate precipitation, polyethylene glycol precipitation).

### Cells

Also provided herein is a cell (e.g., a peripheral mammalian cell, a mammalian hepatocyte, e.g., a human hepatocyte) that includes any of the expression vectors, pairs of expression vectors, nucleic acids, or pairs of nucleic acids described herein. Also provided is a cell (e.g., a mammalian cell, a mammalian hepatocyte, e.g. a human hepatocyte) that is transduced with any of the expression vectors or pairs of expression vectors described herein, edited using lentiviral or CRISPR technologies, or otherwise engineered or modified to express any of the polypeptides or protein complexes described herein. Skilled practitioners will appreciate that the expression vectors and nucleic acids described herein can be introduced into any cell (e.g., any mammalian cell, any hepatocyte) and that a variety of technologies can be utilized for modifying the genome of cells (e.g., mammalian cells). Non-limiting examples of expression vectors and methods for introducing expression vectors and nucleic acids into cells (e.g., any mammalian cell, any hepatocyte, e.g., a human hepatocyte) are described herein.

In some embodiments, the cell is a mammalian cell. In some embodiments, the mammalian cell is a human cell, a rodent cell (e.g., a rat cell or a mouse cell), a rabbit cell, a dog cell, a cat cell, a porcine cell, or a non-human primate cell. In some embodiments, the cell is a hepatocyte. In some embodiments, the cell is present in a subject (e.g., a mammalian subject, a human subject). In some embodiments, the cell is an autologous cell obtained from a subject (e.g., a mammalian subject, a human subject) and cultured ex vivo. In some embodiments, the cell is in vitro.

Without further description, it is believed that one of ordinary skill in the art can, using the preceding description and the following illustrative examples, make and utilize the expression vectors of the present invention and practice the claimed methods. The following working examples specifically point out various aspects of the present invention, and are not to be construed as limiting in any way the remainder of the disclosure.

### Compositions and Kits

Also provided herein are compositions (e.g., pharmaceutical compositions) that include any of the transferrin receptor-binding domains, polypeptides, antibodies, antibody fragments, expression vectors, nucleic acids, and cells containing the same as described herein. Any of the pharmaceutical compositions can include any of the transferrin receptor-binding domains, polypeptides, antibodies, or antibody fragments described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the expression vectors or nucleic acids described herein into a cell (e.g., a mammalian cell, a hepatocyte) (e.g., a liposome or cationic lipid).

In some embodiments, any of the expression vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

The pharmaceutical compositions provided herein can be, e.g., formulated to be compatible with their intended route of administration. In some embodiments, the compositions are formulated for subcutaneous, intramuscular, intravenous, or intrahepatic administration. In some examples, the compositions include a therapeutically effective amount of any of the polypeptides, protein complexes, or expression vectors described herein. Single or multiple administrations of any of the pharmaceutical compositions described herein can be given (e.g., administered) to a subject depending on, for example, the frequency and the dosage required and tolerated by the subject. A dosage of the pharmaceutical composition including any of the polypeptides described herein, any of the protein complexes described herein, or any of the expression vectors described herein should provide a sufficient quantity to effectively ameliorate or treat symptoms, conditions or diseases.

Also provided are kits that include any of the compositions (e.g., pharmaceutical compositions) described herein that include any of the nucleic acids, any of the transferrin receptor-binding domains, any of the polypeptides, any of the antibodies, any of the antibody fragments, any of the expression vectors, or any cells containing the same as described herein. In some embodiments, a kit can include a solid composition (e.g., a lyophilized composition including any of the expression vectors, polypeptides, or protein complexes described herein) and a liquid for solubilizing the lyophilized composition.

In some embodiments, a kit can include at least one dose of any of the compositions (e.g., any of the pharmaceutical compositions) described herein. In some embodiments, a kit can include a pre-loaded syringe including any of the pharmaceutical compositions described herein. In some embodiments, the kit includes a vial including any of the pharmaceutical compositions described herein (e.g., formulated as an aqueous pharmaceutical composition). In some embodiments, the kit can include instructions for performing any of the methods described herein.

### EXAMPLES

### Example 1: Generation of Camelid Antibodies Against Human Transferrin Receptor

A set of experiments was performed to generate camelid antibodies against human transferrin receptor. In these experiments, Creative Biolabs (Shirley, New York) carried out llama immunization, peripheral blood mononuclear cells (PBMCs) isolation, and VHH phage library construction. Briefly, Llama was immunized with the recombinant human transferrin receptor (“TfR”)-apical domain every three weeks over a three month period. Serum titers were measured against the human TfR-apical domain to confirm immune response. On the final day of immunization schedule (after final serum titer confirmation), PBMCs were isolated to purify total RNA to construct a VHH phage library in the pComb3xss phagemid.

### Phage Selection to Isolate Anti-TfR VHH Antibodies

A set of experiments was performed to isolate anti-TfR VHH antibodies from a VHH phage library. The first round of phage panning using the VHH immune phage library was done on the human TfR (full-length) followed by the second round of phage panning on the human TfR-apical or Cynomolgus (“cyno”) TfR-apical domain. After two rounds of phage panning, TG1 E. coli was infected with the phage eluate and plated overnight to isolate individual E. coli colonies. Phage from individual colonies were grown in 96-well plates by adding M13 helper phage in the culture medium and incubating overnight at 30° C. Phage ELISA was carried out on human and cyno TfR (full-length), and human TfR apical domain. A counter screen was also performed in parallel to remove any non-specific binding to streptavidin. Sanger sequencing was done to sequence VHH from the clones that had positive binding on human and cyno TfR.

### Generation of VHH Antibodies

The variable heavy domains of TfR binders identified from the phage panning of immune llama libraries were sub-cloned onto the Fc domain of human IgG1. The expression plasmids contained a CMV promoter to drive expression and a signal peptide for secretion. The heavy chain plasmid was transfected in Expi293 cells. Supernatants were collected after five days and monoclonal antibodies were purified by protein A chromatography. Affinity variants and humanization variants of B07 were sub-cloned onto the Fc domain of human IgG1. These variants were expressed and purified in the same manner. A select set of B07 and humanized B07 variants were expressed as a VHH domain alone. These variants were also expressed and purified in the same manner.

### Example 2: Cell Binding and Uptake Experiments Following Treatment with VHH Antibodies

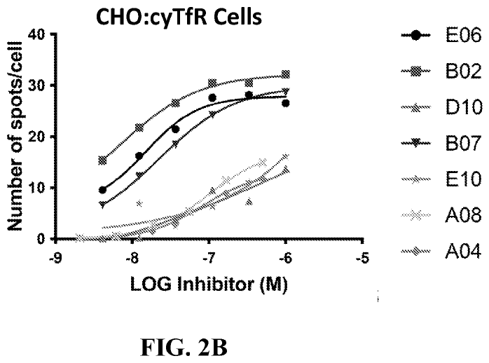

A set of experiments was performed to assess cell binding and uptake of a set of VHH antibodies. In these experiments, HEK293T, CHO:cyTfR, and CHO cells were plated at 40,000 cells/well of 96 well plates in standard growth media (DMEM (Gibco™ Cat. No. 11995073)+10% FBS (VWR Cat No. 89510-188)+1× Pencillin/Streptomycin (Gibco Cat. No. 15140122). Approximately 24 hours later, molecules were diluted into standard growth media warmed to 37° C. Old media was removed from the cells, and the diluted molecules were added to the cells. Cells were incubated at 37° C. for 45 minutes. Cells were then washed with PBS and then fixed for 10 minutes in 4% PFA (Electron Microscopy Sciences Cat. No. 15714-S). Cells were washed with PBS and then blocked with 5% BSA, 0.3% Triton X100 in PBS for 30 minutes. Cells were stained with anti-human IgG-488 (1:1000; Jackson Immuno Research Cat No. 109-545-003), cell mask (1:10,000; Thermo Fisher Cat. No. H32721), and DAPI (1:2000; Thermo Fisher Cat. No. D1306) diluted in 1% BSA, 0.3% Triton X100 in PBS for at least 30 minutes. Cells were washed with PBS, imaged on an Opera Phenix, and images were analyzed with Harmony software. FIGS. 2A and B illustrate exemplary hTfR binding curves generated from the VHH antibodies generated as described in Example 1.

### Example 3: Assessment of Binding Affinity of VHH-Fc Fusions

A set of experiments was performed to assess the binding affinities of VHH-Fc fusions for human and cyno TfR apical domain. In these experiments, binding affinities were determined by surface plasmon resonance using a Biacore™ 8K instrument in 1×HBS-EP+running buffer (GE Healthcare Cat. No. BR100669). Biacore™ Series S CM5 sensor chips were treated with a Human antibody capture kit (GE Healthcare, Cat. No. BR100839). VHH-Fc fusions were captured on each flow cell and serial 3-fold dilutions of human and cyno TfR apical domain (300, 100, 33, 11, 3.7, and 0 nM) were injected at a flow rate of 30 μL/min using the multi cycle kinetics method. Each sample was analyzed with a 270-second association and a 10-minute dissociation. Some VHH-Fc fusions were measured with serial 3-fold dilutions of human and cyno TfR apical domain (100, 33, 11, and 0 nM) with a 300-second association and 10-minute dissociation. After each cycle, the chip was regenerated using 3 M magnesium chloride for 30 seconds at 50 μL/minute. Binding response was corrected by subtracting the RU from a reference flow cell. A 1:1 Langmuir model of simultaneous fitting of kₒₙ and k_{off} was used for kinetics analysis using Biacore™ 8K Evaluation Software. Table 1 shows binding of various VHH clones generated as described in Example 1 (huTfR-AD=human transferrin receptor apical domain; cyTfR-AD=cyno transferrin receptor apical domain).

TABLE 1

| VHH | VHH-Fc | VHH | | | | huTfR-AD | cyTfR-AD |
|---|---|---|---|---|---|---|---|
| Clone | sequence | sequence | CDR1 | CDR2 | CDR3 | K_{D} (M) | K_{D} (M) |
| B07 | SEQ ID | SEQ ID | SEQ ID | SEQ ID | SEQ ID | 1.04 × 10⁻⁹ | 3.13 × 10⁻⁹ |
| | NO: 104 | NO: 40 | NO: 198 | NO: 199 | NO: 200 | | |
| E06 | SEQ ID | SEQ ID | SEQ ID | SEQ ID | SEQ ID | 3.20 × 10⁻⁷ | 1.34 × 10⁻⁶ |
| | NO: 216 | NO: 210 | NO: 207 | NO: 208 | NO: 209 | | |
| B02 | SEQ ID | SEQ ID | SEQ ID | SEQ ID | SEQ ID | 9.85 × 10⁻⁷ | 2.50 × 10⁻⁶ |
| | NO: 217 | NO: 211 | NO: 201 | NO: 202 | NO: 203 | | |
| D10 | SEQ ID | SEQ ID | SEQ ID | SEQ ID | SEQ ID | 6.41 × 10⁻⁸ | 2.85 × 10⁻⁷ |
| | NO: 218 | NO: 212 | NO: 204 | NO: 205 | NO: 206 | | |
| E10 | SEQ ID | SEQ ID | SEQ ID | SEQ ID | SEQ ID | 4.13 × 10⁻¹⁰ | 2.07 × 10⁻⁹ |
| | NO: 219 | NO: 213 | NO: 186 | NO: 189 | NO: 192 | | |
| A08 | SEQ ID | SEQ ID | SEQ ID | SEQ ID | SEQ ID | 2.72 × 10⁻⁹ | 1.83 × 10⁻⁸ |
| | NO: 220 | NO: 214 | NO: 187 | NO: 190 | NO: 193 | | |
| A04 | SEQ ID | SEQ ID | SEQ ID | SEQ ID | SEQ ID | 5.79 × 10⁻⁹ | 1.18 × 10⁻⁸ |
| | NO: 221 | NO: 215 | NO: 188 | NO: 191 | NO: 194 | | |

### Example 4: Generating Affinity Variants of B07

To generate affinity variants of B07, select residues in the CDR regions were mutated with mutation listed in Table 2 with Kabat numbering. The variable domains containing the mutation were sub-cloned onto the corresponding constant heavy domains of human IgG1. The heavy chain plasmids were transfected in Expi293 cells. Supernatants were collected after five days and monoclonal antibodies were purified by protein A chromatography. In order to assess affinities of the B07 mutants for human and cyno TfR apical domain, binding affinities were determined by surface plasmon resonance using a Biacore™ 8K instrument as described herein. Table 2 shows the impact of each mutation in B07 on binding affinity for human and cyno TfR-apical domain.

TABLE 2

| | | | huTfR- | cyTfR- |
|---|---|---|---|---|
| | Affinity | | AD KD | AD KD |
| SEQ ID NO: | variant | Mutation | (nM) | (nM) |
| SEQ ID NO: 40 | WT B07 | | 0.8 | 2.0 |
| SEQ ID NO: 55 | B07v1 | L27F | 1.0 | 2.8 |
| SEQ ID NO: 56 | B07v2 | S29F | 0.9 | 2.5 |
| SEQ ID NO: 57 | B07v3 | D30S | 0.7 | 1.9 |
| SEQ ID NO: 58 | B07v4 | D30E | 0.8 | 2.1 |
| SEQ ID NO: 59 | B07v5 | G32Y | 4.7 | 17.0 |
| SEQ ID NO: 60 | B07v6 | G33A | 36.0 | 120.0 |
| SEQ ID NO: 61 | B07v7 | G35S | 1.2 | 3.2 |
| SEQ ID NO: 62 | B07v8 | T52S | 3.2 | 8.9 |
| SEQ ID NO: 63 | B07v9 | W52aS | 2.7 | 8.7 |
| SEQ ID NO: 64 | B07v10 | W52aG | 3.6 | 11.0 |
| SEQ ID NO: 65 | B07v11 | S53N | 21.0 | 53.0 |
| SEQ ID NO: 66 | B07v12 | R55S | 8.3 | 25.0 |
| SEQ ID NO: 67 | B07v13 | R55G | 8.1 | 22.0 |
| SEQ ID NO: 68 | B07v14 | L58Y | 8.2 | 9.5 |
| SEQ ID NO: 69 | B07v15 | L94R | 3.1 | 10.0 |
| DC2676 | B07v16 | D95A | NB | NB |
| SEQ ID NO: 70 | B07v17 | V96A | 1.0 | 2.9 |
| SEQ ID NO: 71 | B07v18 | V97A | 5.5 | 18.0 |
| SEQ ID NO: 72 | B07v19 | G98A | 330.0 | 160.0 |
| SEQ ID NO: 73 | B07v20 | I99A | 20.0 | 55.0 |
| SEQ ID NO: 74 | B07v21 | G100A | 220.0 | 370.0 |
| SEQ ID NO: 75 | B07v22 | I100aA | 8.9 | 28.0 |
| SEQ ID NO: 76 | B07v23 | E100bA | 2.0 | 5.0 |
| SEQ ID NO: 77 | B07v24 | V100cA | 1.0 | 2.0 |
| SEQ ID NO: 78 | B07v25 | Q100dA | 1.1 | 2.8 |
| SEQ ID NO: 79 | B07v26 | T100eA | 2.4 | 6.6 |
| SEQ ID NO: 80 | B07v27 | Y100fF | 2.8 | 6.7 |
| SEQ ID NO: 81 | B07v28 | G54A | 1.0 | 2.3 |
| SEQ ID NO: 82 | B07v29 | H56A | 5.1 | 11.0 |
| SEQ ID NO: 83 | B07v30 | W52aF | 1.2 | 1 |

NB = No binding

### Example 5: Humanization of B07

A set of experiments was performed to assess impact of humanization on B07 VHH antibodies. In these experiments, B07 VHH was humanized by first aligning the heavy chain variable domain to human antibody germline consensus frameworks. The human H3 consensus sequence was identified as having the highest homology to the B07 framework, so the H3 sequence was as the starting point for humanization. B07 CDRs were grafted into the human H3 consensus framework, and then designed to contain additional variants in which combinations of specific amino acid positions were back-mutated to the original llama residue (37F, 44E, 45R, 47F, 73K, 78V; Kabat numbering). All variants were generated as fusions to a human IgG1 Fc domain as described previously. Variants were expressed recombinantly and tested for binding to human and cyno TfR. Table 3 shows the impact of humanization on the B07 VHH antibodies.

TABLE 3

| | | | | | | | | | huTfR- | cy TfR- |
|---|---|---|---|---|---|---|---|---|---|---|
| SEQ ID | Humanization | | | | | | | | AD KD | AD KD |
| NO: | Variant | Framework | 37F | 44E | 45R | 47F | 73K | 78V | (nM) | (nM) |
| SEQ ID | WT B07 | Llama | ✓ | ✓ | ✓ | ✓ | ✓ | ✓ | 0.8 | 2.0 |
| NO: 40 | | | | | | | | | | |
| DC2644 | hB07v1 | hu VH3 | | | | | | | NB | NB |
| SEQ ID | hB07v2 | hu VH3 | ✓ | | | | | | 4.0 | 9.0 |
| NO: 90 | | | | | | | | | | |
| SEQ ID | hB07v3 | hu VH3 | ✓ | ✓ | | | | | 4.0 | 9.5 |
| NO: 91 | | | | | | | | | | |
| SEQ ID | hB07v4 | hu VH3 | ✓ | | ✓ | | | | 7.0 | 13.4 |
| NO: 92 | | | | | | | | | | |
| SEQ ID | hB07v5 | hu VH3 | ✓ | ✓ | ✓ | | | | 6.7 | 13.8 |
| NO: 93 | | | | | | | | | | |
| DC2649 | hB07v6 | hu VH3 | ✓ | ✓ | ✓ | ✓ | | | NT | NT |
| SEQ ID | hB07v7 | hu VH3 | ✓ | ✓ | ✓ | ✓ | | ✓ | 1.0 | 2.8 |
| NO: 94 | | | | | | | | | | |
| SEQ ID | hB07v8 | hu VH3 | ✓ | ✓ | ✓ | ✓ | ✓ | ✓ | 0.7 | 1.9 |
| NO: 95 | | | | | | | | | | |
| SEQ ID | hB07v9 | hu VH3 | ✓ | | | | ✓ | ✓ | 1.3 | 1.5 |
| NO: 96 | | | | | | | | | | |
| SEQ ID | hB07v10 | hu VH3 | ✓ | ✓ | | | | ✓ | 1.3 | 3.1 |
| NO: 97 | | | | | | | | | | |
| SEQ ID | hB07v11 | hu VH3 | ✓ | | ✓ | | | ✓ | 2.0 | 4.2 |
| NO: 98 | | | | | | | | | | |
| SEQ ID | hB07v12 | hu VH3 | ✓ | ✓ | ✓ | | | ✓ | 2.0 | 4.3 |
| NO: 99 | | | | | | | | | | |
| SEQ ID | hB07v14 | hu VH3 | ✓ | | | | | ✓ | 1.3 | 2.9 |
| NO: 100 | | | | | | | | | | |
| SEQ ID | hB07v15 | hu VH3 | ✓ | ✓ | | | ✓ | ✓ | 0.8 | 1.9 |
| NO: 101 | | | | | | | | | | |
| SEQ ID | hB07v16 | hu VH3 | ✓ | | ✓ | | ✓ | ✓ | 1.1 | 3.3 |
| NO: 102 | | | | | | | | | | |
| SEQ ID | hB07v17 | hu VH3 | ✓ | ✓ | ✓ | | ✓ | ✓ | 0.4 | 2.3 |
| NO: 103 | | | | | | | | | | |

NB = No binding
NT = Not tested

hB07v17 was further humanized in the CDR H1 region. Additional humanization variants were generated containing reversions of CDR H1 residues to the human VH3 consensus H1 sequence. Mutations at position 47 and 52a (Kabat numbering) were also tested. Table 4 shows a summary of mutations made to hB07v17. All variants were generated as fusions to a human IgG1 Fc domain as described herein. Variants were expressed recombinantly and tested for binding to human and cyno TfR. Table 4 shows the impact of humanization in the CDR H1 region on binding affinity of hB07v17 to human and cyno TfR.

TABLE 4

| | Humanized | | | | | | | | | | | | | | |
|---|---|---|---|---|---|---|---|---|---|---|---|---|---|---|---|
| | H1 Variant | | | | | | | | | | | | CDR | huTfR- | cyTfR- |
| SEQ ID | Kabat | CDR H1 | | | | | | | | | | FW2 | H2 | AD KD | AD KD |
| NO: | position | 26 | 27 | 28 | 29 | 30 | 31 | 32 | 33 | 34 | 35 | 47 | W52a | (nM) | (nM) |
| | Human VH3 | G | F | T | F | S | S | Y | A | M | S | W | S | | |
| | consensus | | | | | | | | | | | | | | |
| SEQ ID | B07 | G | L | T | S | D | T | G | G | M | G | F | W | 0.9 | 2.2 |
| NO: 40 | | | | | | | | | | | | | | | |
| SEQ ID | hB07v17 | G | L | T | S | D | T | G | G | M | G | W | W | 1.2 | 2.5 |
| NO: 103 | | | | | | | | | | | | | | | |
| SEQ ID | hB07v18 | G | F | T | F | S | T | G | G | M | G | W | W | 2.6 | 5.0 |
| NO: 134 | | | | | | | | | | | | | | | |
| SEQ ID | hB07v19 | G | F | T | F | S | T | G | G | M | S | W | W | 17 | 32 |
| NO: 135 | | | | | | | | | | | | | | | |
| SEQ ID | hB07v20 | G | F | T | F | S | S | G | G | M | S | W | W | 25 | 47 |
| NO: 136 | | | | | | | | | | | | | | | |
| SEQ ID | hB07v21 | G | F | T | F | S | T | G | G | M | S | W | F | 14 | 25 |
| NO: 137 | | | | | | | | | | | | | | | |
| SEQ ID | hB07v22 | G | F | T | F | S | T | G | G | M | S | W | Y | 37 | 77 |
| NO: 138 | | | | | | | | | | | | | | | |

### Example 6: Generation of B07-GBA Fusion Polypeptides

A set of experiments was performed to assess a fusion polypeptide including a B07 VHH domain or variation thereof fused to a glucosylceramidase beta (GBA) polypeptide. In these experiments, a B07 VHH domain or a humanized B07 VHH domain was fused to the N-terminus or C-terminus of human GBA. These constructs were used for in vitro GBA activity experiments. Additional combinations of B07 variants fused to GBA with various linker lengths were also generated.

A non-limiting example of a nucleic acid sequence encoding a human GBA polypeptide includes SEQ ID NO: 178. A non-limiting example of an amino acid sequence for GBA includes SEQ ID NO: 179.

For in vitro GBA activity experiments, a GBA polypeptide comprising SEQ ID NO: 179 was used as a control.

In a non-limiting example, a B07-GBA fusion polypeptide was created. The amino acid sequence of an exemplary B07-PGRN fusion polypeptide includes SEQ ID NO: 139.

In a non-limiting example, a B07-GBA fusion polypeptide with a 2×GGGGS linker between the B07 and GBA domains was created. The amino acid sequence of an exemplary B07-GBA fusion polypeptide includes SEQ ID NO: 140.

In a non-limiting example, constructs were also created with an N-terminal GBA in a GBA-B07 fusion polypeptide. The amino acid sequence of an exemplary GBA-B07 fusion polypeptide includes SEQ ID NO: 141.

In a non-limiting example, a GBA-B07 fusion polypeptide with a 2×GGGGS linker between the GBA and B07 was created. The amino acid sequence of an GBA-B07 fusion polypeptide includes SEQ ID NO: 142.

In a non-limiting example, a humanzed B07-GBA fusion polypeptide was created. The amino acid sequence of an exemplary humanized B07-GBA fusion polypeptide includes SEQ ID NO: 143.

In a non-limiting example, a GBA-humanzed B07 fusion polypeptide was created. The amino acid sequence of an exemplary GBA-humanized B07 fusion polypeptide includes SEQ ID NO: 144.

### Example 7: Generation of B07-Progranulin Fusion Polypeptides

A set of experiments was performed to assess a fusion polypeptide including a B07 VHH domain or a variant thereof fused to a granulin precursor (progranulin or PGRN) polypeptide. In these experiments, a B07 domain was fused to the N-terminus of human PGRN with two GGGGS linkers.

A non-limiting example of a nucleic acid sequence encoding a human progranulin or granulin precursor (PRGN) polypeptide includes SEQ ID NO: 180. A non-limiting example of an amino acid sequence for human PGRN includes SEQ ID NO: 181. In a non-limiting example, a B07-PGRN construct was created. The amino acid sequence of an exemplary B07-PGRN fusion polypeptide includes SEQ ID NO: 145.

In a non-limiting example, a 6×His-Tev-B07-PGRN construct was created. The addition of a 6×His tag enables purification by nickel chromatography. The amino acid sequence of an exemplary 6×His-Tev-B07-PGRN fusion polypeptide includes SEQ ID NO: 146.

In a non-limiting example, a B07 (N68T)-PGRN construct was created. The N68T mutation in B07 restores the protein A binding site and enables purification by protein A chromatography. The amino acid sequence of an exemplary B07 (N68T)-PGRN fusion polypeptide includes SEQ ID NO: 148.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

Sequence Appendix

| SEQ | | | |
|---|---|---|---|
| ID NO: | Molecule | Region | Sequence |
| SEQ ID | First | | G-X₁-T-X₂-X₃-X₄-X₅-X₆-M-X₇ |
| NO: 1 | sequence | | |
| | consensus | | |
| SEQ ID | Second | | A-I-X₈-X₉-X₁₀-X₁₁-X₁₂-X₁₃-T-X₁₄-Y-A-D-S-V-K-G |
| NO: 2 | sequence | | |
| | consensus | | |
| SEQ ID | Third | | A-X₁₅-D-X₁₆-X₁₇-X₁₈-X₁₉-X₂₀-X₂₁-X₂₂-X₂₃-X₂₄-X₂₅-X₂₆- |
| NO: 3 | sequence | | D-Y |
| | consensus | | |
| SEQ ID | VHH- | Sequence 1 | GLTSDTGGMG |
| NO: 4 | B07 | | |
| SEQ ID | VHH- | Sequence 1 | GFTSDTGGMG |
| NO: 5 | B07_affinity- | | |
| | v1 | | |
| SEQ ID | VHH- | Sequence 1 | GLTFDTGGMG |
| NO: 6 | B07_affinity- | | |
| | v2 | | |
| SEQ ID | VHH- | Sequence 1 | GLTSSTGGMG |
| NO: 7 | B07_affinity- | | |
| | v3 | | |
| SEQ ID | VHH- | Sequence 1 | GLTSETGGMG |
| NO: 8 | B07_affinity- | | |
| | v4 | | |
| SEQ ID | VHH- | Sequence 1 | GLTSDTYGMG |
| NO: 9 | B07_affinity- | | |
| | v5 | | |
| SEQ ID | VHH- | Sequence 1 | GLTSDTGAMG |
| NO: 10 | B07_affinity- | | |
| | v6 | | |
| SEQ ID | VHH- | Sequence 1 | GLTSDTGGMS |
| NO: 11 | B07_affinity- | | |
| | v7 | | |
| SEQ ID | VHH- | Sequence 1 | GFTFSTGGMG |
| NO: 12 | hB07_18 | | |
| SEQ ID | VHH- | Sequence 1 | GFTFSTGGMS |
| NO: 13 | hB07_19 | | |
| SEQ ID | VHH- | Sequence 1 | GFTFSSGGMS |
| NO: 14 | hB07_20 | | |
| SEQ ID | VHH-B07 | Sequence 2 | AITWSGRHTLYADSVKG |
| NO: 15 | | | |
| SEQ ID | VHH- | Sequence 2 | AISWSGRHTLYADSVKG |
| NO: 16 | B07_affinity- | | |
| | v8 | | |
| SEQ ID | VHH- | Sequence 2 | AITSSGRHTLYADSVKG |
| NO: 17 | B07_affinity- | | |
| | v9 | | |
| SEQ ID | VHH- | Sequence 2 | AITGSGRHTLYADSVKG |
| NO: 18 | B07_affinity- | | |
| | v10 | | |
| SEQ ID | VHH- | Sequence 2 | AITWNGRHTLYADSVKG |
| NO: 19 | B07_affinity- | | |
| | v11 | | |
| SEQ ID | VHH- | Sequence 2 | AITWSGSHTLYADSVKG |
| NO: 20 | B07_affinity- | | |
| | v12 | | |
| SEQ ID | VHH- | Sequence 2 | AITWSGGHTLYADSVKG |
| NO: 21 | B07_affinity- | | |
| | v13 | | |
| SEQ ID | VHH- | Sequence 2 | AITWSGRHTYYADSVKG |
| NO: 22 | B07_affinity- | | |
| | v14 | | |
| SEQ ID | VHH- | Sequence 2 | AITWSARHTLYADSVKG |
| NO: 23 | B07_affinity- | | |
| | v28 | | |
| SEQ ID | VHH- | Sequence 2 | AITWSGRATLYADSVKG |
| NO: 24 | B07_affinity- | | |
| | v29 | | |
| SEQ ID | VHH- | Sequence 2 | AITFSGRHTLYADSVKG |
| NO: 25 | B07_affinity- | | |
| | v30 | | |
| SEQ ID | VHH- | Sequence 2 | AITYSGRHTLYADSVKG |
| NO: 26 | hB07_22 | | |
| SEQ ID | VHH-B07 | Sequence 3 | ALDVVGIGIEVQTYDY |
| NO: 27 | | | |
| SEQ ID | VHH- | Sequence 3 | ARDVVGIGIEVQTYDY |
| NO: 28 | B07_affinity- | | |
| | v15 | | |
| SEQ ID | VHH- | Sequence 3 | ALDAVGIGIEVQTYDY |
| NO: 29 | B07_affinity- | | |
| | v17 | | |
| SEQ ID | VHH- | Sequence 3 | ALDVAGIGIEVQTYDY |
| NO: 30 | B07_affinity- | | |
| | v18 | | |
| SEQ ID | VHH- | Sequence 3 | ALDVVAIGIEVQTYDY |
| NO: 31 | B07_affinity- | | |
| | v19 | | |
| SEQ ID | VHH- | Sequence 3 | ALDVVGAGIEVQTYDY |
| NO: 32 | B07_affinity- | | |
| | v20 | | |
| SEQ ID | VHH- | Sequence 3 | ALDVVGIAIEVQTYDY |
| NO: 33 | B07_affinity- | | |
| | v21 | | |
| SEQ ID | VHH- | Sequence 3 | ALDVVGIGAEVQTYDY |
| NO: 34 | B07_affinity- | | |
| | v22 | | |
| SEQ ID | VHH- | Sequence 3 | ALDVVGIGIAVQTYDY |
| NO: 35 | B07_affinity- | | |
| | v23 | | |
| SEQ ID | VHH- | Sequence 3 | ALDVVGIGIEAQTYDY |
| NO: 36 | B07_affinity- | | |
| | v24 | | |
| SEQ ID | VHH- | Sequence 3 | ALDVVGIGIEVATYDY |
| NO: 37 | B07_affinity- | | |
| | v25 | | |
| SEQ ID | VHH- | Sequence 3 | ALDVVGIGIEVQAYDY |
| NO: 38 | B07_affinity- | | |
| | v26 | | |
| SEQ ID | VHH- | Sequence 3 | ALDVVGIGIEVQTFDY |
| NO: 39 | B07_affinity- | | |
| | v27 | | |
| SEQ ID | VHH-B07 | VHH | QVQLVESGGGLVQTGGSLRLSCTASGLTSDTGGM |
| NO: 40 | | | GWFRQAAGKERDFVSAITWSGRHTLYADSVKGRF |
| | | | NISRDKGKNTVFLQMNSLKPEDTAVYYCALDVVG |
| | | | IGIEVQTYDYWGQGTQVTVSS |
| SEQ ID | VHH- | VHH | EVQLVESGGGLVQPGGSLRLSCAASGLTSDTGGM |
| NO: 41 | hB07_2 | | GWFRQAPGKGLEWVSAITWSGRHTLYADSVKGR |
| | | | FTISRDNSKNTLYLQMNSLRAEDTAVYYCALDVV |
| | | | GIGIEVQTYDYWGQGTLVTVSS |
| SEQ ID | VHH- | VHH | EVQLVESGGGLVQPGGSLRLSCAASGLTSDTGGM |
| NO: 42 | hB07_3 | | GWFRQAPGKELEWVSAITWSGRHTLYADSVKGRF |
| | | | TISRDNSKNTLYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 43 | hB07_4 | | GWFRQAPGKGREWVSAITWSGRHTLYADSVKGR |
| | | | FTISRDNSKNTLYLQMNSLRAEDTAVYYCALDVV |
| | | | GIGIEVQTYDYWGQGTLVTVSS |
| SEQ ID | VHH- | VHH | EVQLVESGGGLVQPGGSLRLSCAASGLTSDTGGM |
| NO: 44 | hB07_5 | | GWFRQAPGKEREWVSAITWSGRHTLYADSVKGRF |
| | | | TISRDNSKNTLYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 45 | hB07_7 | | GWFRQAPGKEREFVSAITWSGRHTLYADSVKGRF |
| | | | TISRDNSKNTVYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 46 | hB07_8 | | GWFRQAPGKEREFVSAITWSGRHTLYADSVKGRF |
| | | | TISRDKSKNTVYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 47 | hB07_9 | | GWFRQAPGKGLEWVSAITWSGRHTLYADSVKGR |
| | | | FTISRDKSKNTVYLQMNSLRAEDTAVYYCALDVV |
| | | | GIGIEVQTYDYWGQGTLVTVSS |
| SEQ ID | VHH- | VHH | EVQLVESGGGLVQPGGSLRLSCAASGLTSDTGGM |
| NO: 48 | hB07_10 | | GWFRQAPGKELEWVSAITWSGRHTLYADSVKGRF |
| | | | TISRDNSKNTVYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 49 | hB07_11 | | GWFRQAPGKGREWVSAITWSGRHTLYADSVKGR |
| | | | FTISRDNSKNTVYLQMNSLRAEDTAVYYCALDVV |
| | | | GIGIEVQTYDYWGQGTLVTVSS |
| SEQ ID | VHH- | VHH | EVQLVESGGGLVQPGGSLRLSCAASGLTSDTGGM |
| NO: 50 | hB07_12 | | GWFRQAPGKEREWVSAITWSGRHTLYADSVKGRF |
| | | | TISRDNSKNTVYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 51 | hB07_14 | | GWFRQAPGKGLEWVSAITWSGRHTLYADSVKGR |
| | | | FTISRDNSKNTVYLQMNSLRAEDTAVYYCALDVV |
| | | | GIGIEVQTYDYWGQGTLVTVSS |
| SEQ ID | VHH- | VHH | EVQLVESGGGLVQPGGSLRLSCAASGLTSDTGGM |
| NO: 52 | hB07_15 | | GWFRQAPGKELEWVSAITWSGRHTLYADSVKGRF |
| | | | TISRDKSKNTVYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGLTSDTGGM |
| NO: 53 | hB07_16 | | GWFRQAPGKGREWVSAITWSGRHTLYADSVKGR |
| | | | FTISRDKSKNTVYLQMNSLRAEDTAVYYCALDVV |
| | | | GIGIEVQTYDYWGQGTLVTVSS |
| SEQ ID | VHH- | VHH | EVQLVESGGGLVQPGGSLRLSCAASGLTSDTGGM |
| NO: 54 | hB07_17 | | GWFRQAPGKEREWVSAITWSGRHTLYADSVKGRF |
| | | | TISRDKSKNTVYLQMNSLRAEDTAVYYCALDVVG |
| | | | IGIEVQTYDYWGQGTLVTVSS |
| SEQ ID | VHH- | VHH | QVQLVESGGGLVQTGGSLRLSCTASGFTSDTGGM |
| NO: 55 | B07_affinity- | | GWFRQAAGKERDFVSAITWSGRHTLYADSVKGRF |
| | v1 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTFDTGGM |
| NO: 56 | B07_affinity- | | GWFRQAAGKERDFVSAITWSGRHTLYADSVKGRF |
| | v2 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSSTGGM |
| NO: 57 | B07_affinity- | | GWFRQAAGKERDFVSAITWSGRHTLYADSVKGRF |
| | v3 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSETGGM |
| NO: 58 | B07_affinity- | | GWFRQAAGKERDFVSAITWSGRHTLYADSVKGRF |
| | v4 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTYGM |
| NO: 59 | B07_affinity- | | GWFRQAAGKERDFVSAITWSGRHTLYADSVKGRF |
| | v5 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGAM |
| NO: 60 | B07_affinity- | | GWFRQAAGKERDFVSAITWSGRHTLYADSVKGRF |
| | v6 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 61 | B07_affinity- | | SWFRQAAGKERDFVSAITWSGRHTLYADSVKGRF |
| | v7 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 62 | B07_affinity- | | GWFRQAAGKERDFVSAISWSGRHTLYADSVKGRF |
| | v8 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 63 | B07_affinity- | | GWFRQAAGKERDFVSAITSSGRHTLYADSVKGRF |
| | v9 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 64 | B07_affinity- | | GWFRQAAGKERDFVSAITGSGRHTLYADSVKGRF |
| | v10 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 65 | B07_affinity- | | GWFRQAAGKERDFVSAITWNGRHTLYADSVKGR |
| | v11 | | FNISRDKGKNTVFLQMNSLKPEDTAVYYCALDVV |
| | | | GIGIEVQTYDYWGQGTQVTVSS |
| SEQ ID | VHH- | VHH | QVQLVESGGGLVQTGGSLRLSCTASGLTSDTGGM |
| NO: 66 | B07_affinity- | | GWFRQAAGKERDFVSAITWSGSHTLYADSVKGRF |
| | v12 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 67 | B07_affinity- | | GWFRQAAGKERDFVSAITWSGGHTLYADSVKGRF |
| | v13 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 68 | B07_affinity- | | GWFRQAAGKERDFVSAITWSGRHTYYADSVKGRF |
| | v14 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 69 | B07_affinity- | | GWFRQAAGKERDFVSAITWSGRHTLYADSVKGRF |
| | v15 | | NISRDKGKNTVFLQMNSLKPEDTAVYYCARDVVG |
| | | | IGIEVQTYDYWGQGTQVTVSS |
| SEQ ID | VHH- | VHH | QVQLVESGGGLVQTGGSLRLSCTASGLTSDTGGM |
| NO: 70 | B07_affinity- | | GWFRQAAGKERDFVSAITWSGRHTLYADSVKGRF |
| | v17 | | NISRDKGKNTVFLQMNSLKPEDTAVYYCALDAVG |
| | | | IGIEVQTYDYWGQGTQVTVSS |
| SEQ ID | VHH- | VHH | QVQLVESGGGLVQTGGSLRLSCTASGLTSDTGGM |
| NO: 71 | B07_affinity- | | GWFRQAAGKERDFVSAITWSGRHTLYADSVKGRF |
| | v18 | | NISRDKGKNTVFLQMNSLKPEDTAVYYCALDVAG |
| | | | IGIEVQTYDYWGQGTQVTVSS |
| SEQ ID | VHH- | VHH | QVQLVESGGGLVQTGGSLRLSCTASGLTSDTGGM |
| NO: 72 | B07_affinity- | | GWFRQAAGKERDFVSAITWSGRHTLYADSVKGRF |
| | v19 | | NISRDKGKNTVFLQMNSLKPEDTAVYYCALDVVA |
| | | | IGIEVQTYDYWGQGTQVTVSS |
| SEQ ID | VHH- | VHH | QVQLVESGGGLVQTGGSLRLSCTASGLTSDTGGM |
| NO: 73 | B07_affinity- | | GWFRQAAGKERDFVSAITWSGRHTLYADSVKGRF |
| | v20 | | NISRDKGKNTVFLQMNSLKPEDTAVYYCALDVVG |
| | | | AGIEVQTYDYWGQGTQVTVSS |
| SEQ ID | VHH- | VHH | QVQLVESGGGLVQTGGSLRLSCTASGLTSDTGGM |
| NO: 74 | B07_affinity- | | GWFRQAAGKERDFVSAITWSGRHTLYADSVKGRF |
| | v21 | | NISRDKGKNTVFLQMNSLKPEDTAVYYCALDVVG |
| | | | IAIEVQTYDYWGQGTQVTVSS |
| SEQ ID | VHH- | VHH | QVQLVESGGGLVQTGGSLRLSCTASGLTSDTGGM |
| NO: 75 | B07_affinity- | | GWFRQAAGKERDFVSAITWSGRHTLYADSVKGRF |
| | v22 | | NISRDKGKNTVFLQMNSLKPEDTAVYYCALDVVG |
| | | | IGAEVQTYDYWGQGTQVTVSS |
| SEQ ID | VHH- | VHH | QVQLVESGGGLVQTGGSLRLSCTASGLTSDTGGM |
| NO: 76 | B07_affinity- | | GWFRQAAGKERDFVSAITWSGRHTLYADSVKGRF |
| | v23 | | NISRDKGKNTVFLQMNSLKPEDTAVYYCALDVVG |
| | | | IGIAVQTYDYWGQGTQVTVSS |
| SEQ ID | VHH- | VHH | QVQLVESGGGLVQTGGSLRLSCTASGLTSDTGGM |
| NO: 77 | B07_affinity- | | GWFRQAAGKERDFVSAITWSGRHTLYADSVKGRF |
| | v24 | | NISRDKGKNTVFLQMNSLKPEDTAVYYCALDVVG |
| | | | IGIEAQTYDYWGQGTQVTVSS |
| SEQ ID | VHH- | VHH | QVQLVESGGGLVQTGGSLRLSCTASGLTSDTGGM |
| NO: 78 | B07_affinity- | | GWFRQAAGKERDFVSAITWSGRHTLYADSVKGRF |
| | v25 | | NISRDKGKNTVFLQMNSLKPEDTAVYYCALDVVG |
| | | | IGIEVATYDYWGQGTQVTVSS |
| SEQ ID | VHH- | VHH | QVQLVESGGGLVQTGGSLRLSCTASGLTSDTGGM |
| NO: 79 | B07_affinity- | | GWFRQAAGKERDFVSAITWSGRHTLYADSVKGRF |
| | v26 | | NISRDKGKNTVFLQMNSLKPEDTAVYYCALDVVG |
| | | | IGIEVQAYDYWGQGTQVTVSS |
| SEQ ID | VHH- | VHH | QVQLVESGGGLVQTGGSLRLSCTASGLTSDTGGM |
| NO: 80 | B07_affinity- | | GWFRQAAGKERDFVSAITWSGRHTLYADSVKGRF |
| | v27 | | NISRDKGKNTVFLQMNSLKPEDTAVYYCALDVVG |
| | | | IGIEVQTFDYWGQGTQVTVSS |
| SEQ ID | VHH- | VHH | QVQLVESGGGLVQTGGSLRLSCTASGLTSDTGGM |
| NO: 81 | B07_affinity- | | GWFRQAAGKERDFVSAITWSARHTLYADSVKGRF |
| | v28 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 82 | B07_affinity- | | GWFRQAAGKERDFVSAITWSGRATLYADSVKGRF |
| | v29 | | NISRDKGKNTVFLQMNSLKPEDTAVYYCALDVVG |
| | | | IGIEVQTYDYWGQGTQVTVSS |
| SEQ ID | VHH- | VHH | QVQLVESGGGLVQTGGSLRLSCTASGLTSDTGGM |
| NO: 83 | B07_affinity- | | GWFRQAAGKERDFVSAITFSGRHTLYADSVKGRF |
| | v30 | | NISRDKGKNTVFLQMNSLKPEDTAVYYCALDVVG |
| | | | IGIEVQTYDYWGQGTQVTVSS |
| SEQ ID | VHH- | VHH | EVQLVESGGGLVQPGGSLRLSCAASGFTFSTGGM |
| NO: 84 | hB07_18 | | GWFRQAPGKEREWVSAITWSGRHTLYADSVKGRF |
| | | | TISRDKSKNTVYLQMNSLRAEDTAVYYCALDVVG |
| | | | IGIEVQTYDYWGQGTLVTVSS |
| SEQ ID | VHH- | VHH | EVQLVESGGGLVQPGGSLRLSCAASGFTFSTGGMS |
| NO: 85 | hB07_19 | | WFRQAPGKEREWVSAITWSGRHTLYADSVKGRFT |
| | | | ISRDKSKNTVYLQMNSLRAEDTAVYYCALDVVGI |
| | | | GIEVQTYDYWGQGTLVTVSS |
| SEQ ID | VHH- | VHH | EVQLVESGGGLVQPGGSLRLSCAASGFTFSSGGMS |
| NO: 86 | hB07_20 | | WFRQAPGKEREWVSAITWSGRHTLYADSVKGRFT |
| | | | ISRDKSKNTVYLQMNSLRAEDTAVYYCALDVVGI |
| | | | GIEVQTYDYWGQGTLVTVSS |
| SEQ ID | VHH- | VHH | EVQLVESGGGLVQPGGSLRLSCAASGFTFSTGGMS |
| NO: 87 | hB07_21 | | WFRQAPGKEREWVSAITFSGRHTLYADSVKGRFTI |
| | | | SRDKSKNTVYLQMNSLRAEDTAVYYCALDVVGIG |
| | | | IEVQTYDYWGQGTLVTVSS |
| SEQ ID | VHH- | VHH | EVQLVESGGGLVQPGGSLRLSCAASGFTFSTGGMS |
| NO: 88 | hB07_22 | | WFRQAPGKEREWVSAITYSGRHTLYADSVKGRFTI |
| | | | SRDKSKNTVYLQMNSLRAEDTAVYYCALDVVGIG |
| | | | IEVQTYDYWGQGTLVTVSS |
| SEQ ID | VHH- | VHH | QVQLVESGGGLVQTGGSLRLSCTASGLTSDTGGM |
| NO: 89 | FC_B07 | | GWFRQAAGKERDFVSAITWSGRHTLYADSVKGRF |
| | | | NISRDKGKNTVFLQMNSLKPEDTAVYYCALDVVG |
| | | | IGIEVQTYDYWGQGTQVTVSSASEPKSSDKTHTCP |
| | | | PCPAPELLGGPSVFLFPPKPKDTLMISRTPEVTCVV |
| | | | VDVSHEDPEVKFNWYVDGVEVHNAKTKPREEQY |
| | | | NSTYRVVSVLTVLHQDWLNGKEYKCKVSNKALP |
| | | | APIEKTISKAKGQPREPQVYTLPPSRDELTKNQVSL |
| | | | TCLVKGFYPSDIAVEWESNGQPENNYKTTPPVLDS |
| | | | DGSFFLYSKLTVDKSRWQQGNVFSCSVMHEALHN |
| | | | HYTQKSLSLSPGK |
| SEQ ID | VHH- | VHH | EVQLVESGGGLVQPGGSLRLSCAASGLTSDTGGM |
| NO: 90 | FC_hB07_ | | GWFRQAPGKGLEWVSAITWSGRHTLYADSVKGR |
| | 2 | | FTISRDNSKNTLYLQMNSLRAEDTAVYYCALDVV |
| | | | GIGIEVQTYDYWGQGTLVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | EVQLVESGGGLVQPGGSLRLSCAASGLTSDTGGM |
| NO: 91 | FC_hB07_ | | GWFRQAPGKELEWVSAITWSGRHTLYADSVKGRF |
| | 3 | | TISRDNSKNTLYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: 92 | FC_hB07_ | | GWFRQAPGKGREWVSAITWSGRHTLYADSVKGR |
| | 4 | | FTISRDNSKNTLYLQMNSLRAEDTAVYYCALDVV |
| | | | GIGIEVQTYDYWGQGTLVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | EVQLVESGGGLVQPGGSLRLSCAASGLTSDTGGM |
| NO: 93 | FC_hB07_ | | GWFRQAPGKEREWVSAITWSGRHTLYADSVKGRF |
| | 5 | | TISRDNSKNTLYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: 94 | FC_hB07_ | | GWFRQAPGKEREFVSAITWSGRHTLYADSVKGRF |
| | 7 | | TISRDNSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: 95 | FC_hB07_ | | GWFRQAPGKEREFVSAITWSGRHTLYADSVKGRF |
| | 8 | | TISRDKSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: 96 | FC_hB07_ | | GWFRQAPGKGLEWVSAITWSGRHTLYADSVKGR |
| | 9 | | FTISRDKSKNTVYLQMNSLRAEDTAVYYCALDVV |
| | | | GIGIEVQTYDYWGQGTLVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | EVQLVESGGGLVQPGGSLRLSCAASGLTSDTGGM |
| NO: 97 | FC_hB07_ | | GWFRQAPGKELEWVSAITWSGRHTLYADSVKGRF |
| | 10 | | TISRDNSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: 98 | FC_hB07_ | | GWFRQAPGKGREWVSAITWSGRHTLYADSVKGR |
| | 11 | | FTISRDNSKNTVYLQMNSLRAEDTAVYYCALDVV |
| | | | GIGIEVQTYDYWGQGTLVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | EVQLVESGGGLVQPGGSLRLSCAASGLTSDTGGM |
| NO: 99 | FC_hB07_ | | GWFRQAPGKEREWVSAITWSGRHTLYADSVKGRF |
| | 12 | | TISRDNSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: | FC_hB07_ | | GWFRQAPGKGLEWVSAITWSGRHTLYADSVKGR |
| 100 | 14 | | FTISRDNSKNTVYLQMNSLRAEDTAVYYCALDVV |
| | | | GIGIEVQTYDYWGQGTLVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | EVQLVESGGGLVQPGGSLRLSCAASGLTSDTGGM |
| NO: | FC_hB07_ | | GWFRQAPGKELEWVSAITWSGRHTLYADSVKGRF |
| 101 | 15 | | TISRDKSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGLTSDTGGM |
| NO: | FC_hB07_ | | GWFRQAPGKGREWVSAITWSGRHTLYADSVKGR |
| 102 | 16 | | FTISRDKSKNTVYLQMNSLRAEDTAVYYCALDVV |
| | | | GIGIEVQTYDYWGQGTLVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | EVQLVESGGGLVQPGGSLRLSCAASGLTSDTGGM |
| NO: | FC_hB07_ | | GWFRQAPGKEREWVSAITWSGRHTLYADSVKGRF |
| 103 | 17 | | TISRDKSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07 | | GWFRQAAGKERDFVSAITWSGRHTLYADSVKGRF |
| 104 | | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGFTSDTGGM |
| NO: | FC_B07_ | | GWFRQAAGKERDFVSAITWSGRHTLYADSVKGRF |
| 105 | affinity-v1 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTFDTGGM |
| NO: | FC_B07_ | | GWFRQAAGKERDFVSAITWSGRHTLYADSVKGRF |
| 106 | affinity-v2 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSSTGGM |
| NO: | FC_B07_ | | GWFRQAAGKERDFVSAITWSGRHTLYADSVKGRF |
| 107 | affinity-v3 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSETGGM |
| NO: | FC_B07_ | | GWFRQAAGKERDFVSAITWSGRHTLYADSVKGRF |
| 108 | affinity-v4 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTYGM |
| NO: | FC_B07_ | | GWFRQAAGKERDFVSAITWSGRHTLYADSVKGRF |
| 109 | affinity-v5 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGAM |
| NO: | FC_B07_ | | GWFRQAAGKERDFVSAITWSGRHTLYADSVKGRF |
| 110 | affinity-v6 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | SWFRQAAGKERDFVSAITWSGRHTLYADSVKGRF |
| 111 | affinity-v7 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAISWSGRHTLYADSVKGRF |
| 112 | affinity-v8 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITSSGRHTLYADSVKGRF |
| 113 | affinity-v9 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITGSGRHTLYADSVKGRF |
| 114 | affinity-v10 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWNGRHTLYADSVKGR |
| 115 | affinity-v11 | | FNISRDKGKNTVFLQMNSLKPEDTAVYYCALDVV |
| | | | GIGIEVQTYDYWGQGTQVTVSSGGGGASEPKSSD |
| | | | KTHTCPPCPAPELLGGPSVFLFPPKPKDTLMISRTPE |
| | | | VTCVVVDVSHEDPEVKFNWYVDGVEVHNAKTKP |
| | | | REEQYNSTYRVVSVLTVLHQDWLNGKEYKCKVS |
| | | | NKALPAPIEKTISKAKGQPREPQVYTLPPSRDELTK |
| | | | NQVSLTCLVKGFYPSDIAVEWESNGQPENNYKTTP |
| | | | PVLDSDGSFFLYSKLTVDKSRWQQGNVFSCSVMH |
| | | | EALHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGSHTLYADSVKGRF |
| 116 | affinity-v12 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGGHTLYADSVKGRF |
| 117 | affinity-v13 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTYYADSVKGRF |
| 118 | affinity-v14 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 119 | affinity-v15 | | NISRDKGKNTVFLQMNSLKPEDTAVYYCARDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 120 | affinity-v17 | | NISRDKGKNTVFLQMNSLKPEDTAVYYCALDAVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 121 | affinity-v18 | | NISRDKGKNTVFLQMNSLKPEDTAVYYCALDVAG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 122 | affinity-v19 | | NISRDKGKNTVFLQMNSLKPEDTAVYYCALDVVA |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 123 | affinity-v20 | | NISRDKGKNTVFLQMNSLKPEDTAVYYCALDVVG |
| | | | AGIEVQTYDYWGQGTQVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 124 | affinity-v21 | | NISRDKGKNTVFLQMNSLKPEDTAVYYCALDVVG |
| | | | IAIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 125 | affinity-v22 | | NISRDKGKNTVFLQMNSLKPEDTAVYYCALDVVG |
| | | | IGAEVQTYDYWGQGTQVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 126 | affinity-v23 | | NISRDKGKNTVFLQMNSLKPEDTAVYYCALDVVG |
| | | | IGIAVQTYDYWGQGTQVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 127 | affinity-v24 | | NISRDKGKNTVFLQMNSLKPEDTAVYYCALDVVG |
| | | | IGIEAQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 128 | affinity-v25 | | NISRDKGKNTVFLQMNSLKPEDTAVYYCALDVVG |
| | | | IGIEVATYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 129 | affinity-v26 | | NISRDKGKNTVFLQMNSLKPEDTAVYYCALDVVG |
| | | | IGIEVQAYDYWGQGTQVTVSSGGGGASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRHTLYADSVKGRF |
| 130 | affinity-v27 | | NISRDKGKNTVFLQMNSLKPEDTAVYYCALDVVG |
| | | | IGIEVQTFDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSARHTLYADSVKGRF |
| 131 | affinity-v28 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITWSGRATLYADSVKGRF |
| 132 | affinity-v29 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | QVQLVESGGGLVQTGGSLRLSCTASGLTSDTGGM |
| NO: | FC_B07_ | | GWFRQAAGKERDFVSAITFSGRHTLYADSVKGRF |
| 133 | affinity-v30 | | NISRDKGKNTVFLQMNSLKPEDTAVYYCALDVVG |
| | | | IGIEVQTYDYWGQGTQVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGFTFSTGGM |
| NO: | FC_hB07_ | | GWFRQAPGKEREWVSAITWSGRHTLYADSVKGRF |
| 134 | 18 | | TISRDKSKNTVYLQMNSLRAEDTAVYYCALDVVG |
| | | | IGIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGFTFSTGGMS |
| NO: | FC_hB07_ | | WFRQAPGKEREWVSAITWSGRHTLYADSVKGRFT |
| 135 | 19 | | ISRDKSKNTVYLQMNSLRAEDTAVYYCALDVVGI |
| | | | GIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGFTFSSGGMS |
| NO: | FC_hB07_ | | WFRQAPGKEREWVSAITWSGRHTLYADSVKGRFT |
| 136 | 20 | | ISRDKSKNTVYLQMNSLRAEDTAVYYCALDVVGI |
| | | | GIEVQTYDYWGQGTLVTVSSGGGGASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | VHH- | VHH | EVQLVESGGGLVQPGGSLRLSCAASGFTFSTGGMS |
| NO: | FC_hB07_ | | WFRQAPGKEREWVSAITFSGRHTLYADSVKGRFTI |
| 137 | 21 | | SRDKSKNTVYLQMNSLRAEDTAVYYCALDVVGIG |
| | | | IEVQTYDYWGQGTLVTVSSGGGGASEPKSSDKTH |
| | | | TCPPCPAPELLGGPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSRDELTKNQV |
| | | | SLTCLVKGFYPSDIAVEWESNGQPENNYKTTPPVL |
| | | | DSDGSFFLYSKLTVDKSRWQQGNVFSCSVMHEAL |
| | | | HNHYTQKSLSLSPGK |
| SEQ ID | VHH- | VHH | EVQLVESGGGLVQPGGSLRLSCAASGFTFSTGGMS |
| NO: | FC_hB07_ | | WFRQAPGKEREWVSAITYSGRHTLYADSVKGRFTI |
| 138 | 22 | | SRDKSKNTVYLQMNSLRAEDTAVYYCALDVVGIG |
| | | | IEVQTYDYWGQGTLVTVSSGGGGASEPKSSDKTH |
| | | | TCPPCPAPELLGGPSVFLFPPKPKDTLMISRTPEVTC |
| | | | VVVDVSHEDPEVKFNWYVDGVEVHNAKTKPREE |
| | | | QYNSTYRVVSVLTVLHQDWLNGKEYKCKVSNKA |
| | | | LPAPIEKTISKAKGQPREPQVYTLPPSRDELTKNQV |
| | | | SLTCLVKGFYPSDIAVEWESNGQPENNYKTTPPVL |
| | | | DSDGSFFLYSKLTVDKSRWQQGNVFSCSVMHEAL |
| | | | HNHYTQKSLSLSPGK |
| SEQ ID | B07- | VHH | QVQLVESGGGLVQTGGSLRLSCTASGLTSDTGGM |
| NO: | VHH- | | GWFRQAAGKERDFVSAITWSGRHTLYADSVKGRF |
| 139 | (G4S)1- | | NISRDKGKNTVFLQMNSLKPEDTAVYYCALDVVG |
| | GBA | | IGIEVQTYDYWGQGTQVTVSSGGGGSARPCIPKSF |
| | | | GYSSVVCVCNATYCDSFDPPTFPALGTFSRYESTRS |
| | | | GRRMELSMGPIQANHTGTGLLLTLQPEQKFQKVK |
| | | | GFGGAMTDAAALNILALSPPAQNLLLKSYFSEEGI |
| | | | GYNIIRVPMASCDFSIRTYTYADTPDDFQLHNFSLP |
| | | | EEDTKLKIPLIHRALQLAQRPVSLLASPWTSPTWLK |
| | | | TNGAVNGKGSLKGQPGDIYHQTWARYFVKFLDA |
| | | | YAEHKLQFWAVTAENEPSAGLLSGYPFQCLGFTPE |
| | | | HQRDFIARDLGPTLANSTHHNVRLLMLDDQRLLLP |
| | | | HWAKVVLTDPEAAKYVHGIAVHWYLDFLAPAKA |
| | | | TLGETHRLFPNTMLFASEACVGSKFWEQSVRLGS |
| | | | WDRGMQYSHSIITNLLYHVVGWTDWNLALNPEG |
| | | | GPNWVRNFVDSPIIVDITKDTFYKQPMFYHLGHFS |
| | | | KFIPEGSQRVGLVASQKNDLDAVALMHPDGSAVV |
| | | | VVLNRSSKDVPLTIKDPAVGFLETISPGYSIHTYLW |
| | | | RRQ |
| SEQ ID | B07- | VHH | QVQLVESGGGLVQTGGSLRLSCTASGLTSDTGGM |
| NO: | VHH- | | GWFRQAAGKERDFVSAITWSGRHTLYADSVKGRF |
| 140 | (G4S)2- | | NISRDKGKNTVFLQMNSLKPEDTAVYYCALDVVG |
| | GBA | | IGIEVQTYDYWGQGTQVTVSSGGGGSGGGGSARP |
| | | | CIPKSFGYSSVVCVCNATYCDSFDPPTFPALGTFSR |
| | | | YESTRSGRRMELSMGPIQANHTGTGLLLTLQPEQK |
| | | | FQKVKGFGGAMTDAAALNILALSPPAQNLLLKSY |
| | | | FSEEGIGYNIIRVPMASCDFSIRTYTYADTPDDFQL |
| | | | HNFSLPEEDTKLKIPLIHRALQLAQRPVSLLASPWT |
| | | | SPTWLKTNGAVNGKGSLKGQPGDIYHQTWARYF |
| | | | VKFLDAYAEHKLQFWAVTAENEPSAGLLSGYPFQ |
| | | | CLGFTPEHQRDFIARDLGPTLANSTHHNVRLLMLD |
| | | | DQRLLLPHWAKVVLTDPEAAKYVHGIAVHWYLD |
| | | | FLAPAKATLGETHRLFPNTMLFASEACVGSKFWE |
| | | | QSVRLGSWDRGMQYSHSIITNLLYHVVGWTDWN |
| | | | LALNPEGGPNWVRNFVDSPIIVDITKDTFYKQPMF |
| | | | YHLGHFSKFIPEGSQRVGLVASQKNDLDAVALMH |
| | | | PDGSAVVVVLNRSSKDVPLTIKDPAVGFLETISPGY |
| | | | SIHTYLWRRQ |
| SEQ ID | GBA- | | ARPCIPKSFGYSSVVCVCNATYCDSFDPPTFPALGT |
| NO: | (G4S)1- | | FSRYESTRSGRRMELSMGPIQANHTGTGLLLTLQP |
| 141 | B07-VHH | | EQKFQKVKGFGGAMTDAAALNILALSPPAQNLLL |
| | | | KSYFSEEGIGYNIIRVPMASCDFSIRTYTYADTPDD |
| | | | FQLHNFSLPEEDTKLKIPLIHRALQLAQRPVSLLAS |
| | | | PWTSPTWLKTNGAVNGKGSLKGQPGDIYHQTWA |
| | | | RYFVKFLDAYAEHKLQFWAVTAENEPSAGLLSGY |
| | | | PFQCLGFTPEHQRDFIARDLGPTLANSTHHNVRLL |
| | | | MLDDQRLLLPHWAKVVLTDPEAAKYVHGIAVHW |
| | | | YLDFLAPAKATLGETHRLFPNTMLFASEACVGSKF |
| | | | WEQSVRLGSWDRGMQYSHSIITNLLYHVVGWTD |
| | | | WNLALNPEGGPNWVRNFVDSPIIVDITKDTFYKQP |
| | | | MFYHLGHFSKFIPEGSQRVGLVASQKNDLDAVAL |
| | | | MHPDGSAVVVVLNRSSKDVPLTIKDPAVGFLETIS |
| | | | PGYSIHTYLWRRQGGGGSQVQLVESGGGLVQTGG |
| | | | SLRLSCTASGLTSDTGGMGWFRQAAGKERDFVSA |
| | | | ITWSGRHTLYADSVKGRFNISRDKGKNTVFLQMN |
| | | | SLKPEDTAVYYCALDVVGIGIEVQTYDYWGQGTQ |
| | | | VTVSS |
| SEQ ID | GBA- | | ARPCIPKSFGYSSVVCVCNATYCDSFDPPTFPALGT |
| NO: | (G4S)2- | | FSRYESTRSGRRMELSMGPIQANHTGTGLLLTLQP |
| 142 | B07-VHH | | EQKFQKVKGFGGAMTDAAALNILALSPPAQNLLL |
| | | | KSYFSEEGIGYNIIRVPMASCDFSIRTYTYADTPDD |
| | | | FQLHNFSLPEEDTKLKIPLIHRALQLAQRPVSLLAS |
| | | | PWTSPTWLKTNGAVNGKGSLKGQPGDIYHQTWA |
| | | | RYFVKFLDAYAEHKLQFWAVTAENEPSAGLLSGY |
| | | | PFQCLGFTPEHQRDFIARDLGPTLANSTHHNVRLL |
| | | | MLDDQRLLLPHWAKVVLTDPEAAKYVHGIAVHW |
| | | | YLDFLAPAKATLGETHRLFPNTMLFASEACVGSKF |
| | | | WEQSVRLGSWDRGMQYSHSIITNLLYHVVGWTD |
| | | | WNLALNPEGGPNWVRNFVDSPIIVDITKDTFYKQP |
| | | | MFYHLGHFSKFIPEGSQRVGLVASQKNDLDAVAL |
| | | | MHPDGSAVVVVLNRSSKDVPLTIKDPAVGFLETIS |
| | | | PGYSIHTYLWRRQGGGGSGGGGSQVQLVESGGGL |
| | | | VQTGGSLRLSCTASGLTSDTGGMGWFRQAAGKER |
| | | | DFVSAITWSGRHTLYADSVKGRFNISRDKGKNTVF |
| | | | LQMNSLKPEDTAVYYCALDVVGIGIEVQTYDYWG |
| | | | QGTQVTVSS |
| SEQ ID | hB07v18- | | EVQLVESGGGLVQPGGSLRLSCAASGFTFSTGGM |
| NO: | (G4S)2- | | GWFRQAPGKEREWVSAITWSGRHTLYADSVKGRF |
| 143 | GBA | | TISRDKSKNTVYLQMNSLRAEDTAVYYCALDVVG |
| | | | IGIEVQTYDYWGQGTLVTVSSGGGGSGGGGSARP |
| | | | CIPKSFGYSSVVCVCNATYCDSFDPPTFPALGTFSR |
| | | | YESTRSGRRMELSMGPIQANHTGTGLLLTLQPEQK |
| | | | FQKVKGFGGAMTDAAALNILALSPPAQNLLLKSY |
| | | | FSEEGIGYNIIRVPMASCDFSIRTYTYADTPDDFQL |
| | | | HNFSLPEEDTKLKIPLIHRALQLAQRPVSLLASPWT |
| | | | SPTWLKTNGAVNGKGSLKGQPGDIYHQTWARYF |
| | | | VKFLDAYAEHKLQFWAVTAENEPSAGLLSGYPFQ |
| | | | CLGFTPEHQRDFIARDLGPTLANSTHHNVRLLMLD |
| | | | DQRLLLPHWAKVVLTDPEAAKYVHGIAVHWYLD |
| | | | FLAPAKATLGETHRLFPNTMLFASEACVGSKFWE |
| | | | QSVRLGSWDRGMQYSHSIITNLLYHVVGWTDWN |
| | | | LALNPEGGPNWVRNFVDSPIIVDITKDTFYKQPMF |
| | | | YHLGHFSKFIPEGSQRVGLVASQKNDLDAVALMH |
| | | | PDGSAVVVVLNRSSKDVPLTIKDPAVGFLETISPGY |
| | | | SIHTYLWRRQ |
| SEQ ID | GBA- | | ARPCIPKSFGYSSVVCVCNATYCDSFDPPTFPALGT |
| NO: | (G4S)2- | | FSRYESTRSGRRMELSMGPIQANHTGTGLLLTLQP |
| 144 | hB07v18 | | EQKFQKVKGFGGAMTDAAALNILALSPPAQNLLL |
| | | | KSYFSEEGIGYNIIRVPMASCDFSIRTYTYADTPDD |
| | | | FQLHNFSLPEEDTKLKIPLIHRALQLAQRPVSLLAS |
| | | | PWTSPTWLKTNGAVNGKGSLKGQPGDIYHQTWA |
| | | | RYFVKFLDAYAEHKLQFWAVTAENEPSAGLLSGY |
| | | | PFQCLGFTPEHQRDFIARDLGPTLANSTHHNVRLL |
| | | | MLDDQRLLLPHWAKVVLTDPEAAKYVHGIAVHW |
| | | | YLDFLAPAKATLGETHRLFPNTMLFASEACVGSKF |
| | | | WEQSVRLGSWDRGMQYSHSIITNLLYHVVGWTD |
| | | | WNLALNPEGGPNWVRNFVDSPIIVDITKDTFYKQP |
| | | | MFYHLGHFSKFIPEGSQRVGLVASQKNDLDAVAL |
| | | | MHPDGSAVVVVLNRSSKDVPLTIKDPAVGFLETIS |
| | | | PGYSIHTYLWRRQGGGGSGGGGSEVQLVESGGGL |
| | | | VQPGGSLRLSCAASGFTFSTGGMGWFRQAPGKER |
| | | | EWVSAITWSGRHTLYADSVKGRFTISRDKSKNTV |
| | | | YLQMNSLRAEDTAVYYCALDVVGIGIEVQTYDY |
| | | | WGQGTLVTVSS |
| SEQ ID | VHH(B07)- | | QVQLVESGGGLVQTGGSLRLSCTASGLTSDTGGM |
| NO: | (G4S)2- | | GWFRQAAGKERDFVSAITWSGRHTLYADSVKGRF |
| 145 | PGRN | | NISRDKGKNTVFLQMNSLKPEDTAVYYCALDVVG |
| | | | IGIEVQTYDYWGQGTQVTVSSEPKSSGGGGSGGG |
| | | | GSTRCPDGQFCPVACCLDPGGASYSCCRPLLDKW |
| | | | PTTLSRHLGGPCQVDAHCSAGHSCIFTVSGTSSCCP |
| | | | FPEAVACGDGHHCCPRGFHCSADGRSCFQRSGNN |
| | | | SVGAIQCPDSQFECPDFSTCCVMVDGSWGCCPMP |
| | | | QASCCEDRVHCCPHGAFCDLVHTRCITPTGTHPLA |
| | | | KKLPAQRTNRAVALSSSVMCPDARSRCPDGSTCC |
| | | | ELPSGKYGCCPMPNATCCSDHLHCCPQDTVCDLIQ |
| | | | SKCLSKENATTDLLTKLPAHTVGDVKCDMEVSCP |
| | | | DGYTCCRLQSGAWGCCPFTQAVCCEDHIHCCPAG |
| | | | FTCDTQKGTCEQGPHQVPWMEKAPAHLSLPDPQA |
| | | | LKRDVPCDNVSSCPSSDTCCQLTSGEWGCCPIPEA |
| | | | VCCSDHQHCCPQGYTCVAEGQCQRGSEIVAGLEK |
| | | | MPARRASLSHPRDIGCDQHTSCPVGQTCCPSLGGS |
| | | | WACCQLPHAVCCEDRQHCCPAGYTCNVKARSCE |
| | | | KEVVSAQPATFLARSPHVGVKDVECGEGHFCHDN |
| | | | QTCCRDNRQGWACCPYRQGVCCADRRHCCPAGF |
| | | | RCAARGTKCLRREAPRWDAPLRDPALRQLL |
| SEQ ID | His-Tev- | | HHHHHHENLYFQGQVQLVESGGGLVQTGGSLRLS |
| NO: | VHH(B07)- | | CTASGLTSDTGGMGWFRQAAGKERDFVSAITWSG |
| 146 | (G4S)2- | | RHTLYADSVKGRFNISRDKGKNTVFLQMNSLKPE |
| | PGRN | | DTAVYYCALDVVGIGIEVQTYDYWGQGTQVTVSS |
| | | | EPKSSGGGGSGGGGSTRCPDGQFCPVACCLDPGG |
| | | | ASYSCCRPLLDKWPTTLSRHLGGPCQVDAHCSAG |
| | | | HSCIFTVSGTSSCCPFPEAVACGDGHHCCPRGFHCS |
| | | | ADGRSCFQRSGNNSVGAIQCPDSQFECPDFSTCCV |
| | | | MVDGSWGCCPMPQASCCEDRVHCCPHGAFCDLV |
| | | | HTRCITPTGTHPLAKKLPAQRTNRAVALSSSVMCP |
| | | | DARSRCPDGSTCCELPSGKYGCCPMPNATCCSDHL |
| | | | HCCPQDTVCDLIQSKCLSKENATTDLLTKLPAHTV |
| | | | GDVKCDMEVSCPDGYTCCRLQSGAWGCCPFTQA |
| | | | VCCEDHIHCCPAGFTCDTQKGTCEQGPHQVPWME |
| | | | KAPAHLSLPDPQALKRDVPCDNVSSCPSSDTCCQL |
| | | | TSGEWGCCPIPEAVCCSDHQHCCPQGYTCVAEGQ |
| | | | CQRGSEIVAGLEKMPARRASLSHPRDIGCDQHTSC |
| | | | PVGQTCCPSLGGSWACCQLPHAVCCEDRQHCCPA |
| | | | GYTCNVKARSCEKEVVSAQPATFLARSPHVGVKD |
| | | | VECGEGHFCHDNQTCCRDNRQGWACCPYRQGVC |
| | | | CADRRHCCPAGFRCAARGTKCLRREAPRWDAPLR |
| | | | DPALRQLL |
| SEQ ID | VHH(B07- | | QVQLVESGGGLVQTGGSLRLSCTASGLTSDTGGM |
| NO: | N68T)- | | GWFRQAAGKERDFVSAITWSGRHTLYADSVKGRF |
| 147 | (G4S)2 | | TISRDKGKNTVFLQMNSLKPEDTAVYYCALDVVG |
| | | | IGIEVQTYDYWGQGTQVTVSS |
| SEQ ID | VHH(B07- | | QVQLVESGGGLVQTGGSLRLSCTASGLTSDTGGM |
| NO: | N68T)- | | GWFRQAAGKERDFVSAITWSGRHTLYADSVKGRF |
| 148 | (G4S)2- | | TISRDKGKNTVFLQMNSLKPEDTAVYYCALDVVG |
| | PGRN | | IGIEVQTYDYWGQGTQVTVSSEPKSSGGGGSGGG |
| | | | GSTRCPDGQFCPVACCLDPGGASYSCCRPLLDKW |
| | | | PTTLSRHLGGPCQVDAHCSAGHSCIFTVSGTSSCCP |
| | | | FPEAVACGDGHHCCPRGFHCSADGRSCFQRSGNN |
| | | | SVGAIQCPDSQFECPDFSTCCVMVDGSWGCCPMP |
| | | | QASCCEDRVHCCPHGAFCDLVHTRCITPTGTHPLA |
| | | | KKLPAQRTNRAVALSSSVMCPDARSRCPDGSTCC |
| | | | ELPSGKYGCCPMPNATCCSDHLHCCPQDTVCDLIQ |
| | | | SKCLSKENATTDLLTKLPAHTVGDVKCDMEVSCP |
| | | | DGYTCCRLQSGAWGCCPFTQAVCCEDHIHCCPAG |
| | | | FTCDTQKGTCEQGPHQVPWMEKAPAHLSLPDPQA |
| | | | LKRDVPCDNVSSCPSSDTCCQLTSGEWGCCPIPEA |
| | | | VCCSDHQHCCPQGYTCVAEGQCQRGSEIVAGLEK |
| | | | MPARRASLSHPRDIGCDQHTSCPVGQTCCPSLGGS |
| | | | WACCQLPHAVCCEDRQHCCPAGYTCNVKARSCE |
| | | | KEVVSAQPATFLARSPHVGVKDVECGEGHFCHDN |
| | | | QTCCRDNRQGWACCPYRQGVCCADRRHCCPAGF |
| | | | RCAARGTKCLRREAPRWDAPLRDPALRQLL |
| SEQ ID | Human | | MMDQARSAFSNLFGGEPLSYTRFSLARQVDGDNS |
| NO: | Mature | | HVEMKLAVDEEENADNNTKANVTKPKRCSGSICY |
| 149 | WildType | | GTIAVIVFFLIGFMIGYLGYCKGVEPKTECERLAGT |
| | TFRC | | ESPVREEPGEDFPAARRLYWDDLKRKLSEKLDSTD |
| | Protein | | FTGTIKLLNENSYVPREAGSQKDENLALYVENQFR |
| | Isoform 1 | | EFKLSKVWRDQHFVKIQVKDSAQNSVIIVDKNGRL |
| | | | VYLVENPGGYVAYSKAATVTGKLVHANFGTKKD |
| | | | FEDLYTPVNGSIVIVRAGKITFAEKVANAESLNAIG |
| | | | VLIYMDQTKFPIVNAELSFFGHAHLGTGDPYTPGF |
| | | | PSFNHTQFPPSRSSGLPNIPVQTISRAAAEKLFGNM |
| | | | EGDCPSDWKTDSTCRMVTSESKNVKLTVSNVLKE |
| | | | IKILNIFGVIKGFVEPDHYVVVGAQRDAWGPGAAK |
| | | | SGVGTALLLKLAQMFSDMVLKDGFQPSRSIIFASW |
| | | | SAGDFGSVGATEWLEGYLSSLHLKAFTYINLDKA |
| | | | VLGTSNFKVSASPLLYTLIEKTMQNVKHPVTGQFL |
| | | | YQDSNWASKVEKLTLDNAAFPFLAYSGIPAVSFCF |
| | | | CEDTDYPYLGTTMDTYKELIERIPELNKVARAAAE |
| | | | VAGQFVIKLTHDVELNLDYERYNSQLLSFVRDLN |
| | | | QYRADIKEMGLSLQWLYSARGDFFRATSRLTTDF |
| | | | GNAEKTDRFVMKKLNDRVMRVEYHFLSPYVSPKE |
| | | | SPFRHVFWGSGSHTLPALLENLKLRKQNNGAFNET |
| | | | LFRNQLALATWTIQGAANALSGDVWDIDNEF |
| SEQ ID | Human | | MIGYLGYCKGVEPKTECERLAGTESPVREEPGEDF |
| NO: | Mature | | PAARRLYWDDLKRKLSEKLDSTDFTGTIKLLNENS |
| 150 | WildType | | YVPREAGSQKDENLALYVENQFREFKLSKVWRDQ |
| | TFRC | | HFVKIQVKDSAQNSVIIVDKNGRLVYLVENPGGYV |
| | Protein | | AYSKAATVTGKLVHANFGTKKDFEDLYTPVNGSI |
| | Isoform 2 | | VIVRAGKITFAEKVANAESLNAIGVLIYMDQTKFPI |
| | | | VNAELSFFGHAHLGTGDPYTPGFPSFNHTQFPPSRS |
| | | | SGLPNIPVQTISRAAAEKLFGNMEGDCPSDWKTDS |
| | | | TCRMVTSESKNVKLTVSNVLKEIKILNIFGVIKGFV |
| | | | EPDHYVVVGAQRDAWGPGAAKSGVGTALLLKLA |
| | | | QMFSDMVLKDGFQPSRSIIFASWSAGDFGSVGATE |
| | | | WLEGYLSSLHLKAFTYINLDKAVLGTSNFKVSASP |
| | | | LLYTLIEKTMQNVKHPVTGQFLYQDSNWASKVEK |
| | | | LTLDNAAFPFLAYSGIPAVSFCFCEDTDYPYLGTT |
| | | | MDTYKELIERIPELNKVARAAAEVAGQFVIKLTHD |
| | | | VELNLDYERYNSQLLSFVRDLNQYRADIKEMGLS |
| | | | LQWLYSARGDFFRATSRLTTDFGNAEKTDRFVMK |
| | | | KLNDRVMRVEYHFLSPYVSPKESPFRHVFWGSGS |
| | | | HTLPALLENLKLRKQNNGAFNETLFRNQLALATW |
| | | | TIQGAANALSGDVWDIDNEF |
| SEQ ID | Human | | MDQTKFPIVNAELSFFGHAHLGTGDPYTPGFPSFN |
| NO: | Mature | | HTQFPPSRSSGLPNIPVQTISRAAAEKLFGNMEGDC |
| 151 | WildType | | PSDWKTDSTCRMVTSESKNVKLTVSNVLKEIKILN |
| | TFRC | | IFGVIKGFVEPDHYVVVGAQRDAWGPGAAKSGVG |
| | Protein | | TALLLKLAQMFSDMVLKDGFQPSRSIIFASWSAGD |
| | Isoform 3 | | FGSVGATEWLEGYLSSLHLKAFTYINLDKAVLGTS |
| | | | NFKVSASPLLYTLIEKTMQNVKHPVTGQFLYQDSN |
| | | | WASKVEKLTLDNAAFPFLAYSGIPAVSFCFCEDTD |
| | | | YPYLGTTMDTYKELIERIPELNKVARAAAEVAGQF |
| | | | VIKLTHDVELNLDYERYNSQLLSFVRDLNQYRADI |
| | | | KEMGLSLQWLYSARGDFFRATSRLTTDFGNAEKT |
| | | | DRFVMKKLNDRVMRVEYHFLSPYVSPKESPFRHV |
| | | | FWGSGSHTLPALLENLKLRKQNNGAFNETLFRNQ |
| | | | LALATWTIQGAANALSGDVWDIDNEF |
| SEQ ID | Human | | MMDQARSAFSNLFGGEPLSYTRFSLARQVDGDNS |
| NO: | Mature | | HVEMKLAVDEEENADNNTKANVTKPKRCSGSICY |
| 152 | Wildtype | | GTIAVIVFFLIGFMIGYLGYCKGVEPKTECERLAGT |
| | TFRC | | ESPVREEPGEDFPAARRLYWDDLKRKLSEKLDSTD |
| | Protein | | FTGTIKLLNENSYVPREAGSQKDENLALYVENQFR |
| | Transcript | | EFKLSKVWRDQHFVKIQVKDSAQNSVIIVDKNGRL |
| | Variant 1 | | VYLVENPGGYVAYSKAATVTGKLVHANFGTKKD |
| | | | FEDLYTPVNGSIVIVRAGKITFAEKVANAESLNAIG |
| | | | VLIYMDQTKFPIVNAELSFFGHAHLGTGDPYTPGF |
| | | | PSFNHTQFPPSRSSGLPNIPVQTISRAAAEKLFGNM |
| | | | EGDCPSDWKTDSTCRMVTSESKNVKLTVSNVLKE |
| | | | IKILNIFGVIKGFVEPDHYVVVGAQRDAWGPGAAK |
| | | | SGVGTALLLKLAQMFSDMVLKDGFQPSRSIIFASW |
| | | | SAGDFGSVGATEWLEGYLSSLHLKAFTYINLDKA |
| | | | VLGTSNFKVSASPLLYTLIEKTMQNVKHPVTGQFL |
| | | | YQDSNWASKVEKLTLDNAAFPFLAYSGIPAVSFCF |
| | | | CEDTDYPYLGTTMDTYKELIERIPELNKVARAAAE |
| | | | VAGQFVIKLTHDVELNLDYERYNSQLLSFVRDLN |
| | | | QYRADIKEMGLSLQWLYSARGDFFRATSRLTTDF |
| | | | GNAEKTDRFVMKKLNDRVMRVEYHFLSPYVSPKE |
| | | | SPFRHVFWGSGSHTLPALLENLKLRKQNNGAFNET |
| | | | LFRNQLALATWTIQGAANALSGDVWDIDNEF |
| SEQ ID | Linker | | (G₄S)ₙ |
| NO: | | | |
| 153 | | | |
| SEQ ID | Linker | | GGGGS |
| NO: | | | |
| 154 | | | |
| SEQ ID | Linker | | GGGGSGGGGS |
| NO: | | | |
| 155 | | | |
| SEQ ID | Linker | | GGGGSGGGGSGGGGS |
| NO: | | | |
| 156 | | | |
| SEQ ID | Linker | | GGGGSGGGGSGGGGSGGGGS |
| NO: | | | |
| 157 | | | |
| SEQ ID | Linker | | KESGSVSSEQLAQFRSLD |
| NO: | | | |
| 158 | | | |
| SEQ ID | Linker | | EGKSSGSGSESKST |
| NO: | | | |
| 159 | | | |
| SEQ ID | Linker | | GGGGGGGG |
| NO: | | | |
| 160 | | | |
| SEQ ID | Linker | | GGGGGG |
| NO: | | | |
| 161 | | | |
| SEQ ID | Linker | | GSAGSAAGSGEF |
| NO: | | | |
| 162 | | | |
| SEQ ID | Linker | | (EAAAK)_{N} where N = 1 to 10 |
| NO: | | | |
| 163 | | | |
| SEQ ID | Linker | | A(EAAAK)_{N} where N = 2 to 5 |
| NO: | | | |
| 164 | | | |
| SEQ ID | Linker | | (Ala-Pro)₇ |
| NO: | | | |
| 165 | | | |
| SEQ ID | HLP | | TGTTTGCTGCTTGCAATGTTTGCCCATTTTAGGG |
| NO: | promoter | | TGGACACAGGACGCTGTGGTTTCTGAGCCAGGG |
| 166 | | | GGCGACTCAGATCCCAGCCAGTGGACTTAGCCC |
| | | | CTGTTTGCTCCTCCGATAACTGGGGTGACCTTGG |
| | | | TTAATATTCACCAGCAGCCTCCCCCGTTGCCCCT |
| | | | CTGGATCCACTGCTTAAATACGGACGAGGACAG |
| | | | GGCCCTGTCTCCTCAGCTTCAGGCACCACCACTG |
| | | | ACCTGGGACAGTGAAT |
| SEQ ID | hAAT | | GGATCTTGCTACCAGTGGAACAGCCACTAAGGA |
| NO: | promoter | | TTCTGCAGTGAGAGCAGAGGGCCAGCTAAGTGG |
| 167 | | | TACTCTCCCAGAGACTGTCTGACTCACGCCACCC |
| | | | CCTCCACCTTGGACACAGGACGCTGTGGTTTCTG |
| | | | AGCCAGGTACAATGACTCCTTTCGGTAAGTGCA |
| | | | GTGGAAGCTGTACACTGCCCAGGCAAAGCGTCC |
| | | | GGGCAGCGTAGGCGGGCGACTCAGATCCCAGCC |
| | | | AGTGGACTTAGCCCCTGTTTGCTCCTCCGATAAC |
| | | | TGGGGTGACCTTGGTTAATATTCACCAGCAGCCT |
| | | | CCCCCGTTGCCCCTCTGGATCCACTGCTTAAATA |
| | | | CGGACGAGGACAGGGCCCTGTCTCCTCAGCTTC |
| | | | AGGCACCACCACTGACCTGGGACAGTGAATGAT |
| | | | CCCCCTGATCTGCGGCC |
| SEQ ID | HCB | | GTTAATCATTAAGTCGTTAATTTTTGTGGCCCTT |
| NO: | promoter | | GCGATGTTTGCTCTGGTTAATAATCTCAGGACAA |
| 168 | | | ACAGAGGTTAATAATTTTCCAGATCTCTCTGAGC |
| | | | AATAGTATAAAAGGCCAGCAGCAGCCTGACCAC |
| | | | ATCTCATCCTC |
| SEQ ID | TTR | | GTCTGTCTGCACATTTCGTAGAGCGAGTGTTCCG |
| NO: | promoter | | ATACTCTAATCTCCCTAGGCAAGGTTCATATTTG |
| 169 | | | TGTAGGTTACTTATTCTCCTTTTGTTGACTAAGTC |
| | | | AATAATCAGAATCAGCAGGTTTGGAGTCAGCTT |
| | | | GGCAGGGATCAGCAGCCTGGGTTGGAAGGAGG |
| | | | GGGTATAAAAGCCCCTTCACCAGGAGAAGCCGT |
| | | | C |
| SEQ ID | Serpin | | GGGGGAGGCTGCTGGTGAATATTAACCAAGGTC |
| NO: | promoter | | ACCCCAGTTATCGGAGGAGCAAACAGGGGCTAA |
| 170 | | | GTCCACGTCTGTCTGCACATTTCGTAGAGCGAGT |
| | | | GTTCCGATACTCTAATCTCCCTAGGCAAGGTTCA |
| | | | TATTTGTGTAGGTTACTTATTCTCCTTTTGTTGAC |
| | | | TAAGTCAATAATCAGAATCAGCAGGTTTGGAGT |
| | | | CAGCTTGGCAGGGATCAGCAGCCTGGGTTGGAA |
| | | | GGAGGGGGTATAAAAGCCCCTTCACCAGGAGAA |
| | | | GCCGTCAAGAGGTAAGGGTTTAAGGGATGGTTG |
| | | | GTTGGTGGGGNATTAATGTTTAATTACCTGGAGC |
| | | | ACCTGCCTGAAATCACTTTTTTTCAGGTTGG |
| SEQ ID | LCAT | | AGATCTCTGGGCCTCAAAATGGAGATGGATCCC |
| NO: | promoter | | AGGTCTTGTGGGACCCTGGGATGTTTGGGGACTT |
| 171 | | | TCTATCTAGCACCCCAGTAGGCCTGTCCTGGCCA |
| | | | GAGAAGACTGGTAGGGGCCGAGTGGGGTTTGAA |
| | | | GGCAGCCGGCCCGGCCCAGCCCAGGAGCGCTAT |
| | | | TTATTGCATATTTATTGTTTGGATGTCACCATCA |
| | | | GAGACGAAGGGAAGGGTAGCCAGGGAGGGAGT |
| | | | CCAGCCCAGCTGCCTGCAGGAGAATCTGGCTCA |
| | | | GTCTACTATGGGCAGGGCCCCCCACCAAGCTGA |
| | | | GCCGAATGGAGACAGCTGAGCTGAGGCCTGACT |
| | | | TTTTCAATAAAACATTGTGTAGTTCTGGGCCTCC |
| | | | TGCTGCCCCGGCTCTGTTTCCCCTGGCGCCAAGA |
| | | | GAAGAAGGCGGAACTGAACCCAGGCCCAGAGC |
| | | | CGGCTCCCTGAGGCTGTGCCCCTTTCCGGCAATC |
| | | | TCTGGCCACAACCCCCACTGGCCAGGCCGTCCCT |
| | | | CCCACTGGCCCTAGGGCCCCTCCCACTCCCACAC |
| | | | CAGATAAGGACAGCCCAGTGCCGTC |
| SEQ ID | ApoH | | AGATCTGAGAGTAGGTGTTTGTCCAAAGTTTATA |
| NO: | promoter | | TGCCAAGGCTGTGAGTGAAACAGGAGCTTCGAT |
| 172 | | | CTTTTGGTGTTCCATCTACAACATACACAAAACA |
| | | | AAAGATGGAGAATGAGAAGTCCAGGCAACCCC |
| | | | GGAAACAACAAGTTTCTGTCAAAAGCAATAATG |
| | | | AACTGTTTGTGCCATTAACAAAAACGTTATGAA |
| | | | GACAGAAACCATCTCCCAAAGATTTCATAACAG |
| | | | AGCCACATAAGTGGAAAGTAAATGATTAAAGAA |
| | | | TGTGGGTCTCAGAGTTCCATTCAAATCATGATAC |
| | | | TTTATCTTCTATTTACAAAGATAAAAGTACACCA |
| | | | GAAAATGGTTAATGTTTAAGCGCTTTCATATTTG |
| | | | GCTCTGTCTTTTTAGCAGACGAAAACCACTTTGG |
| | | | CAG |
| SEQ ID | MVM | | AAGAGGTAAGGGTTTAAGGGATGGTTGGTTGGT |
| NO: | promoter | | GGGGNATTAATGTTTAATTACCTGGAGCACCTG |
| 173 | | | CCTGAAATCACTTTTTTTCAGGTTGG |
| SEQ ID | ApoE | | CAGGCTCAGAGGCACACAGGAGTTTCTGGGCTC |
| NO: | | | ACCCTGCCCCCTTCCAACCCCTCAGTTCCCATCC |
| 174 | | | TCCAGCAGCTGTTTGTGTGCTGCCTCTGAAGTCC |
| | | | ACACTGAACAAACTTCAGCCTACTCATGTCCCTA |
| | | | AAATGGGCAAACATTGCAAGCAGCAAACAGCA |
| | | | AACACACAGCCCTCCCTGCCTGCTGACCTTGGA |
| | | | GCTGGGGCAGAGGTCAGAGACCTCTCTGGGCCC |
| | | | ATGCCACCTCCAACATCCACTCGACCCCTTGGAA |
| | | | TTTCGGTGGAGAGGAGCAGAGGTTGTCCTGGCG |
| | | | TGGTTTAGGTAGTGTGAGAGGGTCCGGG |
| SEQ ID | Liver | | GGGGGAGGCTGCTGGTGAATATTAACCAAGGTC |
| NO: | specific | | ACCCCAGTTATCGGAGGAGCAAACAGGGGCTAA |
| 175 | enhancer | | GTCCAC |
| | (104) | | |
| SEQ ID | Secretion | | MEFSSPSREECPKPLSRVSIMAGSLTGLLLLQAVSW |
| NO: | signal | | ASG |
| 176 | peptide | | |
| SEQ ID | Secretion | | MGWSCIILFLVATATGAYA |
| NO: | signal | | |
| 177 | peptide | | |
| SEQ ID | Human | | GCCCGCCCCTGCATCCCTAAAAGCTTCGGCTACA |
| NO: | gluco- | | GCTCGGTGGTGTGTGTCTGCAATGCCACATACTG |
| 178 | sylceramidase | | TGACTCCTTTGACCCCCCGACCTTTCCTGCCCTT |
| | beta | | GGTACCTTCAGCCGCTATGAGAGTACACGCAGT |
| | (GBA) | | GGGCGACGGATGGAGCTGAGTATGGGGCCCATC |
| | variant 1 | | CAGGCTAATCACACGGGCACAGGCCTGCTACTG |
| | nucleic | | ACCCTGCAGCCAGAACAGAAGTTCCAGAAAGTG |
| | acid | | AAGGGATTTGGAGGGGCCATGACAGATGCTGCT |
| | sequence | | GCTCTCAACATCCTTGCCCTGTCACCCCCTGCCC |
| | fo | | AAAATTTGCTACTTAAATCGTACTTCTCTGAAGA |
| | NP_000148.2 | | AGGAATCGGATATAACATCATCCGGGTACCCAT |
| | | | GGCCAGCTGTGACTTCTCCATCCGCACCTACACC |
| | | | TATGCAGACACCCCTGATGATTTCCAGTTGCACA |
| | | | ACTTCAGCCTCCCAGAGGAAGATACCAAGCTCA |
| | | | AGATACCCCTGATTCACCGAGCCCTGCAGTTGG |
| | | | CCCAGCGTCCCGTTTCACTCCTTGCCAGCCCCTG |
| | | | GACATCACCCACTTGGCTCAAGACCAATGGAGC |
| | | | GGTGAATGGGAAGGGGTCACTCAAGGGACAGCC |
| | | | CGGAGACATCTACCACCAGACCTGGGCCAGATA |
| | | | CTTTGTGAAGTTCCTGGATGCCTATGCTGAGCAC |
| | | | AAGTTACAGTTCTGGGCAGTGACAGCTGAAAAT |
| | | | GAGCCTTCTGCTGGGCTGTTGAGTGGATACCCCT |
| | | | TCCAGTGCCTGGGCTTCACCCCTGAACATCAGCG |
| | | | AGACTTCATTGCCCGTGACCTAGGTCCTACCCTC |
| | | | GCCAACAGTACTCACCACAATGTCCGCCTACTC |
| | | | ATGCTGGATGACCAACGCTTGCTGCTGCCCCACT |
| | | | GGGCAAAGGTGGTACTGACAGACCCAGAAGCA |
| | | | GCTAAATATGTTCATGGCATTGCTGTACATTGGT |
| | | | ACCTGGACTTTCTGGCTCCAGCCAAAGCCACCCT |
| | | | AGGGGAGACACACCGCCTGTTCCCCAACACCAT |
| | | | GCTCTTTGCCTCAGAGGCCTGTGTGGGCTCCAAG |
| | | | TTCTGGGAGCAGAGTGTGCGGCTAGGCTCCTGG |
| | | | GATCGAGGGATGCAGTACAGCCACAGCATCATC |
| | | | ACGAACCTCCTGTACCATGTGGTCGGCTGGACC |
| | | | GACTGGAACCTTGCCCTGAACCCCGAAGGAGGA |
| | | | CCCAATTGGGTGCGTAACTTTGTCGACAGTCCCA |
| | | | TCATTGTAGACATCACCAAGGACACGTTTTACA |
| | | | AACAGCCCATGTTCTACCACCTTGGCCACTTCAG |
| | | | CAAGTTCATTCCTGAGGGCTCCCAGAGAGTGGG |
| | | | GCTGGTTGCCAGTCAGAAGAACGACCTGGACGC |
| | | | AGTGGCACTGATGCATCCCGATGGCTCTGCTGTT |
| | | | GTGGTCGTGCTAAACCGCTCCTCTAAGGATGTGC |
| | | | CTCTTACCATCAAGGATCCTGCTGTGGGCTTCCT |
| | | | GGAGACAATCTCACCTGGCTACTCCATTCACACC |
| | | | TACCTGTGGCGTCGCCAG |
| SEQ ID | Human | | ARPCIPKSFGYSSVVCVCNATYCDSFDPPTFPALGT |
| NO: | gluco- | | FSRYESTRSGRRMELSMGPIQANHTGTGLLLTLQP |
| 179 | sylceramidase | | EQKFQKVKGFGGAMTDAAALNILALSPPAQNLLL |
| | beta | | KSYFSEEGIGYNIIRVPMASCDFSIRTYTYADTPDD |
| | (GBA) | | FQLHNFSLPEEDTKLKIPLIHRALQLAQRPVSLLAS |
| | variant 1 | | PWTSPTWLKTNGAVNGKGSLKGQPGDIYHQTWA |
| | amino | | RYFVKFLDAYAEHKLQFWAVTAENEPSAGLLSGY |
| | acid | | PFQCLGFTPEHQRDFIARDLGPTLANSTHHNVRLL |
| | sequence | | MLDDQRLLLPHWAKVVLTDPEAAKYVHGIAVHW |
| | NP_000148.2 | | YLDFLAPAKATLGETHRLFPNTMLFASEACVGSKF |
| | | | WEQSVRLGSWDRGMQYSHSIITNLLYHVVGWTD |
| | | | WNLALNPEGGPNWVRNFVDSPIIVDITKDTFYKQP |
| | | | MFYHLGHFSKFIPEGSQRVGLVASQKNDLDAVAL |
| | | | MHPDGSAVVVVLNRSSKDVPLTIKDPAVGFLETIS |
| | | | PGYSIHTYLWRRQ |
| SEQ ID | Human | | ACGCGGTGCCCAGATGGTCAGTTCTGCCCTGTG |
| NO: | granulin | | GCCTGCTGCCTGGACCCCGGAGGAGCCAGCTAC |
| 180 | precursor | | AGCTGCTGCCGTCCCCTTCTGGACAAATGGCCCA |
| | (progranulin) | | CAACACTGAGCAGGCATCTGGGTGGCCCCTGCC |
| | (PGRN) | | AGGTTGATGCCCACTGCTCTGCCGGCCACTCCTG |
| | nucleic | | CATCTTTACCGTCTCAGGGACTTCCAGTTGCTGC |
| | acid | | CCCTTCCCAGAGGCCGTGGCATGCGGGGATGGC |
| | sequence | | CATCACTGCTGCCCACGGGGCTTCCACTGCAGTG |
| | (NM_002087.3) | | CAGACGGGCGATCCTGCTTCCAAAGATCAGGTA |
| | | | ACAACTCCGTGGGTGCCATCCAGTGCCCTGATA |
| | | | GTCAGTTCGAATGCCCGGACTTCTCCACGTGCTG |
| | | | TGTTATGGTCGATGGCTCCTGGGGGTGCTGCCCC |
| | | | ATGCCCCAGGCTTCCTGCTGTGAAGACAGGGTG |
| | | | CACTGCTGTCCGCACGGTGCCTTCTGCGACCTGG |
| | | | TTCACACCCGCTGCATCACACCCACGGGCACCC |
| | | | ACCCCCTGGCAAAGAAGCTCCCTGCCCAGAGGA |
| | | | CTAACAGGGCAGTGGCCTTGTCCAGCTCGGTCA |
| | | | TGTGTCCGGACGCACGGTCCCGGTGCCCTGATG |
| | | | GTTCTACCTGCTGTGAGCTGCCCAGTGGGAAGT |
| | | | ATGGCTGCTGCCCAATGCCCAACGCCACCTGCT |
| | | | GCTCCGATCACCTGCACTGCTGCCCCCAAGACA |
| | | | CTGTGTGTGACCTGATCCAGAGTAAGTGCCTCTC |
| | | | CAAGGAGAACGCTACCACGGACCTCCTCACTAA |
| | | | GCTGCCTGCGCACACAGTGGGGGATGTGAAATG |
| | | | TGACATGGAGGTGAGCTGCCCAGATGGCTATAC |
| | | | CTGCTGCCGTCTACAGTCGGGGGCCTGGGGCTG |
| | | | CTGCCCTTTTACCCAGGCTGTGTGCTGTGAGGAC |
| | | | CACATACACTGCTGTCCCGCGGGGTTTACGTGTG |
| | | | ACACGCAGAAGGGTACCTGTGAACAGGGGCCCC |
| | | | ACCAGGTGCCCTGGATGGAGAAGGCCCCAGCTC |
| | | | ACCTCAGCCTGCCAGACCCACAAGCCTTGAAGA |
| | | | GAGATGTCCCCTGTGATAATGTCAGCAGCTGTCC |
| | | | CTCCTCCGATACCTGCTGCCAACTCACGTCTGGG |
| | | | GAGTGGGGCTGCTGTCCAATCCCAGAGGCTGTC |
| | | | TGCTGCTCGGACCACCAGCACTGCTGCCCCCAG |
| | | | GGCTACACGTGTGTAGCTGAGGGGCAGTGTCAG |
| | | | CGAGGAAGCGAGATCGTGGCTGGACTGGAGAA |
| | | | GATGCCTGCCCGCCGGGCTTCCTTATCCCACCCC |
| | | | AGAGACATCGGCTGTGACCAGCACACCAGCTGC |
| | | | CCGGTGGGGCAGACCTGCTGCCCGAGCCTGGGT |
| | | | GGGAGCTGGGCCTGCTGCCAGTTGCCCCATGCT |
| | | | GTGTGCTGCGAGGATCGCCAGCACTGCTGCCCG |
| | | | GCTGGCTACACCTGCAACGTGAAGGCTCGATCC |
| | | | TGCGAGAAGGAAGTGGTCTCTGCCCAGCCTGCC |
| | | | ACCTTCCTGGCCCGTAGCCCTCACGTGGGTGTGA |
| | | | AGGACGTGGAGTGTGGGGAAGGACACTTCTGCC |
| | | | ATGATAACCAGACCTGCTGCCGAGACAACCGAC |
| | | | AGGGCTGGGCCTGCTGTCCCTACCGCCAGGGCG |
| | | | TCTGTTGTGCTGATCGGCGCCACTGCTGTCCTGC |
| | | | TGGCTTCCGCTGCGCAGCCAGGGGTACCAAGTG |
| | | | TTTGCGCAGGGAGGCCCCGCGCTGGGACGCCCC |
| | | | TTTGAGGGACCCAGCCTTGAGACAGCTGCTGTG |
| | | | A |
| SEQ ID | Human | | TRCPDGQFCPVACCLDPGGASYSCCRPLLDKWPTT |
| NO: | granulin | | LSRHLGGPCQVDAHCSAGHSCIFTVSGTSSCCPFPE |
| 181 | precursor | | AVACGDGHHCCPRGFHCSADGRSCFQRSGNNSVG |
| | (progranulin | | AIQCPDSQFECPDFSTCCVMVDGSWGCCPMPQAS |
| | or | | CCEDRVHCCPHGAFCDLVHTRCITPTGTHPLAKKL |
| | PGRN) | | PAQRTNRAVALSSSVMCPDARSRCPDGSTCCELPS |
| | variant 1 | | GKYGCCPMPNATCCSDHLHCCPQDTVCDLIQSKC |
| | amino | | LSKENATTDLLTKLPAHTVGDVKCDMEVSCPDGY |
| | acid | | TCCRLQSGAWGCCPFTQAVCCEDHIHCCPAGFTC |
| | sequence | | DTQKGTCEQGPHQVPWMEKAPAHLSLPDPQALKR |
| | NP_002078.1 | | DVPCDNVSSCPSSDTCCQLTSGEWGCCPIPEAVCC |
| | | | SDHQHCCPQGYTCVAEGQCQRGSEIVAGLEKMPA |
| | | | RRASLSHPRDIGCDQHTSCPVGQTCCPSLGGSWAC |
| | | | CQLPHAVCCEDRQHCCPAGYTCNVKARSCEKEVV |
| | | | SAQPATFLARSPHVGVKDVECGEGHFCHDNQTCC |
| | | | RDNRQGWACCPYRQGVCCADRRHCCPAGFRCAA |
| | | | RGTKCLRREAPRWDAPLRDPALRQLL |
| SEQ ID | 6X His | | HHHHHHENLYFQG |
| NO: | Tev Tag | | |
| 182 | | | |
| SEQ ID | Consensus | | G-X₁-T-L-D-X₂-X₃-A-I-X₄ (SEQ ID NO: 183), wherein |
| NO: | for CDR1 | | X₁ is S or F, wherein X₂ is D, H, or Y, X₃ is Y or F, |
| 183 | for E10, | | and X₄ is G or A |
| | A08, and | | |
| | A04 | | |
| SEQ ID | Consensus | | C-I-S-X₅-X₆-D-G-X₇-T-X₈-Y-X₉-D-X₁₀-V-K-G (SEQ ID |
| NO: | for CDR2 | | NO: 184), wherein X₅ is S or R, X₆ is S or G, X₇ is I or |
| 184 | for E10, | | R, X₈ is F or Y, X₉ is A or G, and X₁₀ is F or S; |
| | A08, and | | |
| | A04 | | |
| SEQ ID | Consensus | | A-X₁₁-X₁₂-X₁₃-G-P-N-X₁₄-C-R-G-W-L-W-X₁₅-P-X₁₆- |
| NO: | for CDR3 | | X₁₇-S-G-S (SEQ ID NO: 185), wherein X₁₁ is A or S, |
| 185 | for E10, | | X₁₂ is K or H, X₁₃ is Y or D, X₁₄ is I or V, X₁₅ is V or |
| | A08 and | | E, X₁₆ is P or Q, and X₁₇ is V, I, or L |
| | A04 | | |
| SEQ ID | CDR1 of | | GSTLDDYAIG |
| NO: | E10 | | |
| 186 | | | |
| SEQ ID | CDR1 of | | GSTLDHYAIG |
| NO: | A08 | | |
| 187 | | | |
| SEQ ID | CDR1 of | | GFTLDYFAIA |
| NO: | A04 | | |
| 188 | | | |
| SEQ ID | CDR2 of | | CISSSDGITFYGDFVKG |
| NO: | E10 | | |
| 189 | | | |
| SEQ ID | CDR2 of | | CISRSDGITYYADSVKG |
| NO: | A08 | | |
| 190 | | | |
| SEQ ID | CDR2 of | | CISSGDGRTFYADSVKG |
| NO: | A04 | | |
| 191 | | | |
| SEQ ID | CDR3 of | | AAKYGPNICRGWLWVPPVSGS |
| NO: | E10 | | |
| 192 | | | |
| SEQ ID | CDR3 of | | AAHYGPNVCRGWLWEPPISGS |
| NO: | A08 | | |
| 193 | | | |
| SEQ ID | CDR3 of | | ASHDGPNVCRGWLWVPQLSGS |
| NO: | A04 | | |
| 194 | | | |
| SEQ ID | Consensus | | G-X₁-T-X₂-X₃-X₄-X₅-X₆-M-X₇, wherein X₁ is L or F, X₂ |
| NO: | sequence | | is S or F, X₃ is D or S or E, X₄ is T or S, X₅ is G or |
| 195 | for CDR1 | | Y, X₆ is G or A, or X₇ is G or S |
| | of B07 | | |
| SEQ ID | Consensus | | A-I-X₈-X₉-X₁₀-X₁₁-X₁₂-X₁₃-T-X₁₄-Y-A-D-S-V-K-G, |
| NO: | sequence | | wherein X₈ is T or S, X₉ is W, S, G, F, or Y, X₁₀ is S |
| 196 | for CDR2 | | or N, X₁₁ is G or A, X₁₂ is R, S, or G, X₁₃ is H or A, |
| | of B07 | | or X₁₄ is L or Y |
| SEQ ID | Consensus | | A-X₁₅-D-X₁₆-X₁₇-X₁₈-X₁₉-X₂₀-X₂₁-X₂₂-X₂₃-X₂₄-X₂₅-X₂₆- |
| NO: | sequence | | D-Y, wherein X₁₅ is L or R, X₁₆ is V or A, X₁₇ is V or A, |
| 197 | of B07 | | X₁₈ is G or A, X₁₉ is I or A, X₂₀ is G or A, X₂₁ is I or |
| | | | A, X₂₂ is E or A, X₂₃ V or A, X₂₄ is Q or A, X₂₅ is T or |
| | | | A, and X₂₆ is Y or F |
| SEQ ID | CDR1 of | | GLTSDTGGMG |
| NO: | B07 | | |
| 198 | | | |
| SEQ ID | CDR2 of | | AITWSGRHTLYADSVKG |
| NO: | B07 | | |
| 199 | | | |
| SEQ ID | CDR3 of | | ALDVVGIGIEVQTYDY |
| NO: | B07 | | |
| 200 | | | |
| SEQ ID | CDR1 of | | ESAFSLNAIG |
| NO: | B02 | | |
| 201 | | | |
| SEQ ID | CDR2 of | | GIGTDGITTYYADFVKD |
| NO: | B02 | | |
| 202 | | | |
| SEQ ID | CDR3 of | | NAGSWRTVLSGTHVSRS |
| NO: | B02 | | |
| 203 | | | |
| SEQ ID | CDR1 of | | GRDYNHFQRA |
| NO: | D10 | | |
| 204 | | | |
| SEQ ID | CDR2 of | | RITWSGTITYNESVKG |
| NO: | D10 | | |
| 205 | | | |
| SEQ ID | CDR3 of | | ALKTQPPLSQDAGDYTY |
| NO: | D10 | | |
| 206 | | | |
| SEQ ID | CDR1 of | | GRSLSTYVMG |
| NO: | E06 | | |
| 207 | | | |
| SEQ ID | CDR2 of | | ARNGMSTYYTDSVKD |
| NO: | E06 | | |
| 208 | | | |
| SEQ ID | CDR3 of | | AGDRSWSRLLRGEYEY |
| NO: | E06 | | |
| 209 | | | |
| SEQ ID | E06 VHH | | QLQLVESGGGLVQPGGSLRLSCAGSGRSLSTYVM |
| NO: | | | GWFRQAPGKERELVAARNGMSTYYTDSVKDRSAI |
| 210 | | | SRDNAKNTVYLEMNSLEPEDTAVYYCAGDRSWS |
| | | | RLLRGEYEYWGQGTQVTVSS |
| SEQ ID | B02 VHH | | QVQLVESGGGLVQAGGSLRLSCAASESAFSLNAIG |
| NO: | | | WYRQAPGNQRELVAGIGTDGITTYYADFVKDRFTI |
| 211 | | | SRDNVKNTVYLQMNSLKPEDTAVYYCNAGSWRT |
| | | | VLSGTHVSRSWGPGTQVTVSS |
| SEQ ID | D10 VHH | | QLQLVESGGGLVKTGGSLRLSCTDSGRDYNHFQR |
| NO: | | | AWFRQAPGKEREFVARITWSGTITYNESVKGRFTI |
| 212 | | | SGDDASNTIHLQMNSLKSDDTAIYYCALKTQPPLS |
| | | | QDAGDYTYWGQGTQVTVSS |
| SEQ ID | E10 VHH | | QVQLVESGGGLVQPGGSLRLSCTTSGSTLDDYAIG |
| NO: | | | WFRQAPGKEREGVSCISSSDGITFYGDFVKGRFTIS |
| 213 | | | RDNAKNSVYLQMNNLKPEDTAVYYCAAKYGPNI |
| | | | CRGWLWVPPVSGSWGQGTQVTVSS |
| SEQ ID | A08 VHH | | QVQLVESGGGVVQPGGSLRLSCAASGSTLDHYAI |
| NO: | | | GWFRQAPGKEREGVSCISRSDGITYYADSVKGRFT |
| 214 | | | ISRDNAKNTVYLQMNSLKPEDTAVYYCAAHYGPN |
| | | | VCRGWLWEPPISGSWGQGTQVTVSS |
| SEQ ID | A04 VHH | | QLQLVESGGGLVQPGESLRLSCVPSGFTLDYFAIA |
| NO: | | | WFRQAPGKEREGVSCISSGDGRTFYADSVKGRFTI |
| 215 | | | SRDNSKNTVYLQMNSLKPEDTAVYYCASHDGPNV |
| | | | CRGWLWVPQLSGSWGQGTQVTVSS |
| SEQ ID | E06 | | QLQLVESGGGLVQPGGSLRLSCAGSGRSLSTYVM |
| NO: | VHH-Fc | | GWFRQAPGKERELVAARNGMSTYYTDSVKDRSAI |
| 216 | | | SRDNAKNTVYLEMNSLEPEDTAVYYCAGDRSWS |
| | | | RLLRGEYEYWGQGTQVTVSSASEPKSSDKTHTCPP |
| | | | CPAPELLGGPSVFLFPPKPKDTLMISRTPEVTCVVV |
| | | | DVSHEDPEVKFNWYVDGVEVHNAKTKPREEQYN |
| | | | STYRVVSVLTVLHQDWLNGKEYKCKVSNKALPAP |
| | | | IEKTISKAKGQPREPQVYTLPPSRDELTKNQVSLTC |
| | | | LVKGFYPSDIAVEWESNGQPENNYKTTPPVLDSDG |
| | | | SFFLYSKLTVDKSRWQQGNVFSCSVMHEALHNHY |
| | | | TQKSLSLSPGK |
| SEQ ID | B02 | | QVQLVESGGGLVQAGGSLRLSCAASESAFSLNAIG |
| NO: | VHH-Fc | | WYRQAPGNQRELVAGIGTDGITTYYADFVKDRFTI |
| 217 | | | SRDNVKNTVYLQMNSLKPEDTAVYYCNAGSWRT |
| | | | VLSGTHVSRSWGPGTQVTVSSASEPKSSDKTHTCP |
| | | | PCPAPELLGGPSVFLFPPKPKDTLMISRTPEVTCVV |
| | | | VDVSHEDPEVKFNWYVDGVEVHNAKTKPREEQY |
| | | | NSTYRVVSVLTVLHQDWLNGKEYKCKVSNKALP |
| | | | APIEKTISKAKGQPREPQVYTLPPSRDELTKNQVSL |
| | | | TCLVKGFYPSDIAVEWESNGQPENNYKTTPPVLDS |
| | | | DGSFFLYSKLTVDKSRWQQGNVFSCSVMHEALHN |
| | | | HYTQKSLSLSPGK |
| SEQ ID | D10 | | QLQLVESGGGLVKTGGSLRLSCTDSGRDYNHFQR |
| NO: | VHH-Fc | | AWFRQAPGKEREFVARITWSGTITYNESVKGRFTI |
| 218 | | | SGDDASNTIHLQMNSLKSDDTAIYYCALKTQPPLS |
| | | | QDAGDYTYWGQGTQVTVSSASEPKSSDKTHTCPP |
| | | | CPAPELLGGPSVFLFPPKPKDTLMISRTPEVTCVVV |
| | | | DVSHEDPEVKFNWYVDGVEVHNAKTKPREEQYN |
| | | | STYRVVSVLTVLHQDWLNGKEYKCKVSNKALPAP |
| | | | IEKTISKAKGQPREPQVYTLPPSRDELTKNQVSLTC |
| | | | LVKGFYPSDIAVEWESNGQPENNYKTTPPVLDSDG |
| | | | SFFLYSKLTVDKSRWQQGNVFSCSVMHEALHNHY |
| | | | TQKSLSLSPGK |
| SEQ ID | E10 | | QVQLVESGGGLVQPGGSLRLSCTTSGSTLDDYAIG |
| NO: | VHH-Fc | | WFRQAPGKEREGVSCISSSDGITFYGDFVKGRFTIS |
| 219 | | | RDNAKNSVYLQMNNLKPEDTAVYYCAAKYGPNI |
| | | | CRGWLWVPPVSGSWGQGTQVTVSSASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| SEQ ID | A08 | | QVQLVESGGGVVQPGGSLRLSCAASGSTLDHYAI |
| NO: | VHH-Fc | | GWFRQAPGKEREGVSCISRSDGITYYADSVKGRFT |
| 220 | | | ISRDNAKNTVYLQMNSLKPEDTAVYYCAAHYGPN |
| | | | VCRGWLWEPPISGSWGQGTQVTVSSASEPKSSDK |
| | | | THTCPPCPAPELLGGPSVFLFPPKPKDTLMISRTPEV |
| | | | TCVVVDVSHEDPEVKFNWYVDGVEVHNAKTKPR |
| | | | EEQYNSTYRVVSVLTVLHQDWLNGKEYKCKVSN |
| | | | KALPAPIEKTISKAKGQPREPQVYTLPPSRDELTKN |
| | | | QVSLTCLVKGFYPSDIAVEWESNGQPENNYKTTPP |
| | | | VLDSDGSFFLYSKLTVDKSRWQQGNVFSCSVMHE |
| | | | ALHNHYTQKSLSLSPGK |
| SEQ ID | A04 | | QLQLVESGGGLVQPGESLRLSCVPSGFTLDYFAIA |
| NO: | VHH-Fc | | WFRQAPGKEREGVSCISSGDGRTFYADSVKGRFTI |
| 221 | | | SRDNSKNTVYLQMNSLKPEDTAVYYCASHDGPNV |
| | | | CRGWLWVPQLSGSWGQGTQVTVSSASEPKSSDKT |
| | | | HTCPPCPAPELLGGPSVFLFPPKPKDTLMISRTPEVT |
| | | | CVVVDVSHEDPEVKFNWYVDGVEVHNAKTKPRE |
| | | | EQYNSTYRVVSVLTVLHQDWLNGKEYKCKVSNK |
| | | | ALPAPIEKTISKAKGQPREPQVYTLPPSRDELTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |

What is claimed is:

1. A transferrin receptor-binding protein comprising a camelid heavy chain variable domain.

2. The transferrin receptor-binding protein of claim 1, wherein the protein binds to human transferrin receptor with a K_{D} of from about 1 nM to about 6 μM.

3. The transferrin receptor-binding protein of claim 1 or 2, wherein the protein binds to cynomolgus transferrin receptor with a K_{D} of from about 1 nM to about 2.5 mM.

4. A transferrin receptor-binding domain, wherein the transferrin receptor-binding domain comprises:
(a) a first sequence of G-X₁-T-X₂-X₃-X₄-X₅-X₆-M-X₇ (SEQ ID NO: 1), wherein X₁ is L or F, X₂ is S or F, X₃ is D or S or E, X₄ is T or S, X₅ is G or Y, X₆ is G or A, or X₇ is G or S;
(b) a second sequence of A-I-X₈-X₉-X₁₀-X₁₁-X₁₂-X₁₃-T-X₁₄-Y-A-D-S-V-K-G (SEQ ID NO: 2), wherein X₈ is T or S, X₉ is W or S or G or F or Y, X₁₀ is S or N, X₁₁ is G or A, X₁₂ is R or S or G, X₁₃ is H or A, or X₁₄ is L or Y; and
(c) a third sequence of A-X₁₅-D-X₁₆-X₁₇-X₁₈-X₁₉-X₂₀-X₂₁-X₂₂-X₂₃-X₂₄-X₂₅-X₂₆-D-Y (SEQ ID NO: 3), wherein X₁₅ is L or R, X₁₆ is V or A, X₁₇ is V or A, X₁₈ is G or A, X₁₉ is I or A, X₂₀ is G or A, X₂₁ is I or A, X₂₂ is E or A, X₂₃ V or A, X₂₄ is Q or A, X₂₅ is T or A, or X₂₆ is Y or F.

5. The transferrin receptor-binding domain of claim 4, wherein the transferrin receptor-binding domain comprises:
(a)
a first sequence selected from the group consist-
ing of:
(SEQ ID NO: 4)
GLTSDTGGMG,
(SEQ ID NO: 12)
GFTFSTGGMG,
(SEQ ID NO: 5)
GFTSDTGGMG,
(SEQ ID NO: 6)
GLTFDTGGMG,
(SEQ ID NO: 7)
GLTSSTGGMG,
(SEQ ID NO: 8)
GLTSETGGMG,
(SEQ ID NO: 9)
GLTSDTYGMG,
(SEQ ID NO: 10)
GLTSDTGAMG,
(SEQ ID NO: 11)
GLTSDTGGMS,
(SEQ ID NO: 13)
GFTFSTGGMS,
and
(SEQ ID NO: 14)
GFTFSSGGMS;
(b)
a second sequence selected from the group consist-
ing of:
(SEQ ID NO: 15)
AITWSGRHTLYADSVKG,
(SEQ ID NO: 16)
AISWSGRHTLYADSVKG,
(SEQ ID NO: 17)
AITSSGRHTLYADSVKG,
(SEQ ID NO: 18)
AITGSGRHTLYADSVKG,
(SEQ ID NO: 19)
AITWNGRHTLYADSVKG,
(SEQ ID NO: 20)
AITWSGSHTLYADSVKG,
(SEQ ID NO: 21)
AITWSGGHTLYADSVKG,
(SEQ ID NO: 22)
AITWSGRHTYYADSVKG,
(SEQ ID NO: 23)
AITWSARHTLYADSVKG,
(SEQ ID NO: 24)
AITWSGRATLYADSVKG,
(SEQ ID NO: 25)
AITFSGRHTLYADSVKG,
and
(SEQ ID NO: 26)
AITYSGRHTLYADSVKG;
and
(c)
a third sequence selected from the group consist-
ing of:
(SEQ ID NO: 27)
ALDVVGIGIEVQTYDY,
(SEQ ID NO: 28)
ARDVVGIGIEVQTYDY,
(SEQ ID NO: 29)
ALDAVGIGIEVQTYDY,
(SEQ ID NO: 30)
ALDVAGIGIEVQTYDY,
(SEQ ID NO: 31)
ALDVVAIGIEVQTYDY,
(SEQ ID NO: 32)
ALDVVGAGIEVQTYDY,
(SEQ ID NO: 33)
ALDVVGIAIEVQTYDY,
(SEQ ID NO: 34)
ALDVVGIGAEVQTYDY,
(SEQ ID NO: 35)
ALDVVGIGIAVQTYDY,
(SEQ ID NO: 36)
ALDVVGIGIEAQTYDY,
(SEQ ID NO: 37)
ALDVVGIGIEVATYDY,
(SEQ ID NO: 38)
ALDVVGIGIEVQAYDY,
and
(SEQ ID NO: 39)
ALDVVGIGIEVQTFDY.

6. The transferrin receptor-binding domain of claim 4 or 5, wherein the transferrin receptor-binding domain comprises:
(a)
(SEQ ID NO: 4)
GLTSDTGGMG,
(SEQ ID NO: 15)
AITWSGRHTLYADSVKG,
and
(SEQ ID NO: 27)
ALDVVGIGIEVQTYDY;
(b)
(SEQ ID NO: 12)
GFTFSTGGMG,
(SEQ ID NO: 15)
AITWSGRHTLYADSVKG,
and
(SEQ ID NO: 27)
ALDVVGIGIEVQTYDY;
(c)
(SEQ ID NO: 5)
GFTSDTGGMG,
(SEQ ID NO: 15)
AITWSGRHTLYADSVKG,
and
(SEQ ID NO: 27)
ALDVVGIGIEVQTYDY;
(d)
(SEQ ID NO: 6)
GLTFDTGGMG,
(SEQ ID NO: 15)
AITWSGRHTLYADSVKG,
and
(SEQ ID NO: 27)
ALDVVGIGIEVQTYDY;
(e)
(SEQ ID NO: 7)
GLTSSTGGMG,
(SEQ ID NO: 15)
AITWSGRHTLYADSVKG,
and
(SEQ ID NO: 27)
ALDVVGIGIEVQTYDY;
(f)
(SEQ ID NO: 8)
GLTSETGGMG,
(SEQ ID NO: 15)
AITWSGRHTLYADSVKG,
and
(SEQ ID NO: 27)
ALDVVGIGIEVQTYDY;
(g)
(SEQ ID NO: 9)
GLTSDTYGMG,
(SEQ ID NO: 15)
AITWSGRHTLYADSVKG,
and
(SEQ ID NO: 27)
ALDVVGIGIEVQTYDY;
(h)
(SEQ ID NO: 10)
GLTSDTGAMG,
(SEQ ID NO: 15)
AITWSGRHTLYADSVKG,
and
(SEQ ID NO: 27)
ALDVVGIGIEVQTYDY;
(i)
(SEQ ID NO: 11)
GLTSDTGGMS,
(SEQ ID NO: 15)
AITWSGRHTLYADSVKG,
and
(SEQ ID NO: 27)
ALDVVGIGIEVQTYDY;
(j)
(SEQ ID NO: 4)
GLTSDTGGMG,
(SEQ ID NO: 16)
AISWSGRHTLYADSVKG,
and
(SEQ ID NO: 27)
ALDVVGIGIEVQTYDY;
(k)
(SEQ ID NO: 4)
GLTSDTGGMG,
(SEQ ID NO: 17)
AITSSGRHTLYADSVKG,
and
(SEQ ID NO: 27)
ALDVVGIGIEVQTYDY;
(l)
(SEQ ID NO: 4)
GLTSDTGGMG,
(SEQ ID NO: 18)
AITGSGRHTLYADSVKG,
and
(SEQ ID NO: 27)
ALDVVGIGIEVQTYDY;
(m)
(SEQ ID NO: 4)
GLTSDTGGMG,
(SEQ ID NO: 19)
AITWNGRHTLYADSVKG,
and
(SEQ ID NO: 27)
ALDVVGIGIEVQTYDY;
(n)
(SEQ ID NO: 4)
GLTSDTGGMG,
(SEQ ID NO: 20)
AITWSGSHTLYADSVKG,
and
(SEQ ID NO: 27)
ALDVVGIGIEVQTYDY;
(o)
(SEQ ID NO: 4)
GLTSDTGGMG,
(SEQ ID NO: 21)
AITWSGGHTLYADSVKG,
and
(SEQ ID NO: 27)
ALDVVGIGIEVQTYDY;
(p)
(SEQ ID NO: 4)
GLTSDTGGMG,
(SEQ ID NO: 22)
AITWSGRHTYYADSVKG,
and
(SEQ ID NO: 27)
ALDVVGIGIEVQTYDY;
(q)
(SEQ ID NO: 4)
GLTSDTGGMG,
(SEQ ID NO: 15)
AITWSGRHTLYADSVKG,
and
(SEQ ID NO: 28)
ARDVVGIGIEVQTYDY;
(r)
(SEQ ID NO: 4)
GLTSDTGGMG,
(SEQ ID NO: 15)
AITWSGRHTLYADSVKG,
and
(SEQ ID NO: 29)
ALDAVGIGIEVQTYDY;
(s)
(SEQ ID NO: 4)
GLTSDTGGMG,
(SEQ ID NO: 15)
AITWSGRHTLYADSVKG,
and
(SEQ ID NO: 30)
ALDVAGIGIEVQTYDY;
(t)
(SEQ ID NO: 4)
GLTSDTGGMG,
(SEQ ID NO: 15)
AITWSGRHTLYADSVKG,
and
(SEQ ID NO: 31)
ALDVVAIGIEVQTYDY;
(u)
(SEQ ID NO: 4)
GLTSDTGGMG,
(SEQ ID NO: 15)
AITWSGRHTLYADSVKG,
and
(SEQ ID NO: 32)
ALDVVGAGIEVQTYDY;
(v)
(SEQ ID NO: 4)
GLTSDTGGMG,
(SEQ ID NO: 15)
AITWSGRHTLYADSVKG,
and
(SEQ ID NO: 33)
ALDVVGIAIEVQTYDY;
(w)
(SEQ ID NO: 4)
GLTSDTGGMG,
(SEQ ID NO: 15)
AITWSGRHTLYADSVKG,
and
(SEQ ID NO: 34)
ALDVVGIGAEVQTYDY;
(x)
(SEQ ID NO: 4)
GLTSDTGGMG,
(SEQ ID NO: 15)
AITWSGRHTLYADSVKG,
and
(SEQ ID NO: 35)
ALDVVGIGIAVQTYDY;
(y)
(SEQ ID NO: 4)
GLTSDTGGMG,
(SEQ ID NO: 15)
AITWSGRHTLYADSVKG,
and
(SEQ ID NO: 36)
ALDVVGIGIEAQTYDY;
(z)
(SEQ ID NO: 4)
GLTSDTGGMG,
(SEQ ID NO: 15)
AITWSGRHTLYADSVKG,
and
(SEQ ID NO: 37)
ALDVVGIGIEVATYDY;
(aa)
(SEQ ID NO: 4)
GLTSDTGGMG,
(SEQ ID NO: 15)
AITWSGRHTLYADSVKG,
and
(SEQ ID NO: 38)
ALDVVGIGIEVQAYDY;
(bb)
(SEQ ID NO: 4)
GLTSDTGGMG,
(SEQ ID NO: 15)
AITWSGRHTLYADSVKG,
and
(SEQ ID NO: 39)
ALDVVGIGIEVQTFDY;
(cc)
(SEQ ID NO: 4)
GLTSDTGGMG,
(SEQ ID NO: 23)
AITWSARHTLYADSVKG,
and
(SEQ ID NO: 27)
ALDVVGIGIEVQTYDY;
(dd)
(SEQ ID NO: 4)
GLTSDTGGMG,
(SEQ ID NO: 24)
AITWSGRATLYADSVKG,
and
(SEQ ID NO: 27)
ALDVVGIGIEVQTYDY;
(ee)
(SEQ ID NO: 4)
GLTSDTGGMG,
(SEQ ID NO: 25)
AITFSGRHTLYADSVKG,
and
(SEQ ID NO: 27)
ALDVVGIGIEVQTYDY;
(ff)
(SEQ ID NO: 13)
GFTFSTGGMS,
(SEQ ID NO: 15)
AITWSGRHTLYADSVKG,
and
(SEQ ID NO: 27)
ALDVVGIGIEVQTYDY;
(gg)
(SEQ ID NO: 14)
GFTFSSGGMS,
(SEQ ID NO: 15)
AITWSGRHTLYADSVKG,
and
(SEQ ID NO: 27)
ALDVVGIGIEVQTYDY;
(hh)
(SEQ ID NO: 13)
GFTFSTGGMS,
(SEQ ID NO: 25)
AITFSGRHTLYADSVKG,
and
(SEQ ID NO: 27)
ALDVVGIGIEVQTYDY;
or
(ii)
(SEQ ID NO: 13)
GFTFSTGGMS,
(SEQ ID NO: 26)
AITYSGRHTLYADSVKG,
and
(SEQ ID NO: 27)
ALDVVGIGIEVQTYDY.

7. A transferrin receptor-binding domain, wherein the transferrin receptor-binding domain comprises:
(a) a first sequence of
(SEQ ID NO: 183)
G-X₁-T-L-D-X₂-X₃-A-I-X₄, wherein X₁ is S or F,
wherein X₂ is D, H, or Y, X₃ is Y or F, and X₄
is G or A;
(b) a second sequence of
(SEQ ID NO: 184)
C-I-S-X₅-X₆-D-G-X₇-T-X₈-Y-X₉-D-X₁₀-V-K-G,
wherein X₅ is S or R, X₆ is S or G, X₇ is I or R,
X₈ is F or Y, X₉ is A or G, and X₁₀ is F or S; and
(c) a third sequence of
(SEQ ID NO: 185)
A-X₁₁-X₁₂-X₁₃-G-P-N-X₁₄-C-R-G-W-L-W-X₁₅-P-X₁₆-X₁₇-S-
G-S, wherein X₁₁ is A or S, X₁₂ is K or H, X₁₃ is
Y or D, X₁₄ is I or V, X₁₅ is V or E, X₁₆ is P or Q,
and X₁₇ is V, I, or L

8. The transferrin receptor-binding domain of claim 7, wherein the transferrin receptor-binding domain comprises:
(a) a first sequence selected from the group
consisting of:
(SEQ ID NO: 186)
GSTLDDYAIG,
(SEQ ID NO: 187)
GSTLDHYAIG,
and
(SEQ ID NO: 188)
GFTLDYFAIA;
(b) a second sequence selected from the group
consisting of:
(SEQ ID NO: 189)
CISSSDGITFYGDFVKG,
(SEQ ID NO: 190)
CISRSDGITYYADSVKG,
and
(SEQ ID NO: 191)
CISSGDGRTFYADSVKG;
and
(c) a third sequence selected from the group
consisting of:
(SEQ ID NO: 192)
AAKYGPNICRGWLWVPPVSGS,
(SEQ ID NO: 193)
AAHYGPNVCRGWLWEPPISGS,
and
(SEQ ID NO: 194)
ASHDGPNVCRGWLWVPQLSGS.

9. The transferrin receptor-binding domain of claim 7 or 8, wherein the transferrin receptor-binding domain comprises:
(a)
(SEQ ID NO: 186)
GSTLDDYAIG,
(SEQ ID NO: 189)
CISSSDGITFYGDFVKG,
and
(SEQ ID NO: 192)
AAKYGPNICRGWLWVPPVSGS;
(b)
(SEQ ID NO: 187)
GSTLDHYAIG,
(SEQ ID NO: 190)
CISRSDGITYYADSVKG,
and
(SEQ ID NO: 193)
AAHYGPNVCRGWLWEPPISGS;
or
(c)
(SEQ ID NO: 188)
GFTLDYFAIA,
(SEQ ID NO: 191)
CISSGDGRTFYADSVKG,
and
(SEQ ID NO: 194)
ASHDGPNVCRGWLWVPQLSGS.

10. A transferrin receptor-binding domain, wherein the transferrin receptor-binding domain comprises:
(a) a first sequence of
(SEQ ID NO: 195)
G-X₁-T-X₂-X₃-X₄-X₅-X₆-M-X₇,
wherein X₁ is L or F, X₂ is S or F, X₃ is D
or S or E, X₄ is T or S, X₅ is G or Y, X₆ is G
or A, and X₇ is G or S;
(b) a second sequence of
(SEQ ID NO: 196)
A-I-X₈-X₉-X₁₀-X₁₁-X₁₂-X₁₃-T-X₁₄-Y-A-D-S-V-K-G,
wherein X₈ is T or S, X₉ is W, S, G, F, or Y, X₁₀
is S or N, X₁₁ is G or A, X₁₂ is R, S, or G, X₁₃ is
H or A, or X₁₄ is L or Y; and
(c) a third sequence of
(SEQ ID NO: 197)
A-X₁₅-D-X₁₆-X₁₇-X₁₈-X₁₉-X₂₀-X₂₁-X₂₂-X₂₃-X₂₄-X₂₅-X₂₆-D-Y,
wherein X₁₅ is L or R, X₁₆ is V or A, X₁₇ is V or
A, X₁₈ is G or A, X₁₉ is I or A, X₂₀ is G or A,
X₂₁ is I or A, X₂₂ is E or A, X₂₃ V or A, X₂₄ is
Q or A, X₂₅ is T or A, and X₂₆ is Y or F.

11. The transferrin receptor-binding domain of claim 10, wherein the transferrin receptor-binding domain comprises: GLTSDTGGMG (SEQ ID NO: 198), AITWSGRHTLYADSVKG (SEQ ID NO: 199), and ALDVVGIGIEVQTYDY (SEQ ID NO: 200).

12. A transferrin receptor-binding domain, wherein the transferrin receptor-binding domain comprises: ESAFSLNAIG (SEQ ID NO: 201), GIGTDGITTYYADFVKD (SEQ ID NO: 202), and NAGSWRTVLSGTHVSRS (SEQ ID NO: 203).

13. A transferrin receptor-binding domain, wherein the transferrin receptor-binding domain comprises: GRDYNHFQRA (SEQ ID NO: 204), RITWSGTITYNESVKG (SEQ ID NO: 205), and ALKTQPPLSQDAGDYTY (SEQ ID NO: 206).

14. A transferrin receptor-binding domain, wherein the transferrin receptor-binding domain comprises: GSTLDDYAIG (SEQ ID NO: 186), CISSSDGITFYGDFVKG (SEQ ID NO: 189), and AAKYGPNICRGWLWVPPVSGS (SEQ ID NO: 192).

15. A transferrin receptor-binding domain, wherein the transferrin receptor-binding domain comprises: GRSLSTYVMG (SEQ ID NO: 210), ARNGMSTYYTDSVKD (SEQ ID NO: 211), and AGDRSWSRLLRGEYEY (SEQ ID NO: 212).

16. A transferrin receptor-binding domain, wherein the transferrin receptor-binding domain comprises: GSTLDHYAIG (SEQ ID NO: 187), CISRSDGITYYADSVKG (SEQ ID NO: 190), and AAHYGPNVCRGWLWEPPISGS (SEQ ID NO: 193).

17. A transferrin receptor-binding domain, wherein the transferrin receptor-binding domain comprises: GFTLDYFAIA (SEQ ID NO: 188), CISSGDGRTFYADSVKG (SEQ ID NO: 191), and ASHDGPNVCRGWLWVPQLSGS (SEQ ID NO: 194).

18. A transferrin receptor-binding domain, wherein the transferrin receptor-binding domain comprises: GLTSDTGGMG (SEQ ID NO: 198), AITWSGRHTLYADSVKG (SEQ ID NO: 199), and ALDVVGIGIEVQTYDY (SEQ ID NO: 200).

19. The transferrin receptor-binding domain of any one of claims 1-18, wherein the transferrin receptor-binding domain is a VHH domain.

20. The transferrin receptor-binding domain of claim 19, wherein the VHH domain is humanized.

21. A polypeptide comprising the transferrin receptor-binding domain of any one of claims 1-20.

22. The polypeptide of claim 21, wherein the polypeptide further comprises a fusion partner.

23. The polypeptide of claim 22, wherein the fusion partner is selected from the group consisting of: an Fc domain, an enzyme, a hormone, a growth factor, a cytokine, a chemokine, a glycolipid, a lipid, a soluble protein, a nucleic acid, and an additional antigen-binding domain.

24. The polypeptide of claim 22 or 23, wherein the polypeptide comprises a linker between the fusion partner and the transferrin receptor-binding domain.

25. The polypeptide of claim 22 or 23, wherein the fusion partner is directly adjacent to the transferrin receptor-binding domain.

26. The polypeptide of any one of claims 21-25, wherein the polypeptide is a single polypeptide.

27. The polypeptide of claim 26, wherein the single polypeptide comprises a sequence at least 80% identical to any one of SEQ ID NOs: 216-221.

28. The polypeptide of claim 27, wherein the single polypeptide comprises a sequence at least 90% identical to any one of SEQ ID NOs: 216-221.

29. The polypeptide of claim 28, wherein the single polypeptide comprises a sequence of any one of SEQ ID NOs: 216-221.

30. The polypeptide of any one of claims 21-29, wherein the polypeptide is part of a protein complex.

31. An antibody comprising the transferrin receptor-binding domain of any one of claims 1-20.

32. The antibody of claim 31, wherein the antibody is humanized.

33. The antibody of claim 31 or 32, further comprising a fusion partner.

34. The antibody of claim 33, wherein the fusion partner is selected from the group consisting of: an enzyme, a hormone, a growth factor, a cytokine, a chemokine, a glycolipid, a lipid, a soluble protein, a nucleic acid, and an additional antigen-binding domain.

35. The antibody of claim 33 or 34, wherein the fusion partner further comprises a linker between the fusion partner and the transferrin receptor-binding domain.

36. The antibody of claim 33 or 34, wherein the fusion partner is directly adjacent to the transferrin receptor-binding domain.

37. An antibody fragment comprising the transferrin receptor-binding domain of any one of claims 1-20.

38. The antibody fragment of claim 37, wherein the antibody fragment comprises a VHH domain.

39. The antibody fragment of claim 37 or 38, wherein the antibody fragment is humanized.

40. The antibody fragment of any one of claims 37-39, further comprising a fusion partner.

41. The antibody fragment of claim 40, wherein the fusion partner is selected from the group consisting of: an enzyme, a hormone, a growth factor, a cytokine, a chemokine, a glycolipid, a lipid, a soluble protein, a nucleic acid, and an additional antigen-binding domain.

42. The antibody fragment of claim 40 or 41, wherein the fusion partner further comprises a linker between the fusion partner and the transferrin receptor-binding domain.

43. The antibody fragment of claim 40 or 41, wherein the fusion partner is directly adjacent to the transferrin receptor-binding domain.

44. A composition comprising (i) the transferrin receptor-binding domain of any one of claims 1-20, the polypeptide of any one of claims 21-30, the antibody of any one of claims 31-36, or the antibody fragment of any one of claims 37-43, and (ii) a pharmaceutically acceptable carrier.

45. A kit comprising a composition of claim 44.

46. A nucleic acid encoding the transferrin receptor-binding domain of any one of claims 1-20, the polypeptide of any one of claims 21-30, the antibody of any one of claims 31-36, or the antibody fragment of any one of claims 37-43.

47. An expression vector comprising the nucleic acid of claim 46.

48. A host cell comprising the nucleic acid of claim 46 or the expression vector of claim 47.

49. A method of producing the transferrin receptor-binding domain, the polypeptide, the antibody, or the antibody fragment comprising:
(a) culturing the host cell of claim 48 in a culture medium under conditions sufficient to allow for the production of the transferrin receptor-binding domain, the polypeptide, the antibody, or the antibody fragment; and
(b) harvesting the transferrin receptor-binding domain, the polypeptide, the antibody, or the antibody fragment from the host cell or the culture medium.

50. The method of claim 49, further comprising isolating the transferrin receptor-binding domain, the polypeptide, the antibody, or the antibody fragment.

51. The method of claim 50, further comprising formulating the isolated transferrin receptor-binding domain, polypeptide, antibody, or antibody fragment.

52. A transferrin receptor-binding domain comprising a sequence that is at least 80% identical to any one of SEQ ID NOs: 40-88, 147, and 210-215.

53. The transferrin receptor-binding domain of claim 52, wherein the transferrin receptor-binding domain comprises a sequence that is at least 90% identical to any one of SEQ ID Nos: 40-88, 147, and 210-215.

54. The transferrin receptor-binding domain of claim 53, wherein the transferrin receptor-binding domain comprises a sequence that is any one of SEQ ID Nos: 40-88, 147, and 210-215.

55. The transferrin receptor-binding domain of any one of claims 52-54, wherein the transferrin receptor-binding domain is a VHH domain.

56. The transferrin receptor-binding domain of claim 55, wherein the VHH domain is humanized.

57. A polypeptide comprising the transferrin receptor-binding domain of any one of claims 52-56.

58. The polypeptide of claim 57, wherein the polypeptide further comprises a fusion partner.

59. The polypeptide of claim 58, wherein the fusion partner is selected from the group consisting of: an Fc domain, an enzyme, a hormone, a growth factor, a cytokine, a chemokine, a glycolipid, a lipid, a soluble protein, a nucleic acid, and an additional antigen-binding domain.

60. The polypeptide of claim 58 or 59, wherein the polypeptide comprises a linker between the fusion partner and the transferrin receptor-binding domain.

61. The polypeptide of claim 58 or 59, wherein the fusion partner is directly adjacent to the transferrin receptor-binding domain.

62. The polypeptide of any one of claims 57-61, wherein the polypeptide is a single polypeptide.

63. The polypeptide of claim 62, wherein the single polypeptide comprises a sequence at least 80% identical to any one of SEQ ID NOs: 216-221.

64. The polypeptide of claim 63, wherein the single polypeptide comprises a sequence at least 90% identical to any one of SEQ ID NOs: 216-221.

65. The polypeptide of claim 64, wherein the single polypeptide comprises a sequence of any one of SEQ ID NOs: 216-221.

66. The polypeptide of any one of claims 57-61, wherein the polypeptide is part of a protein complex.

67. An antibody comprising the transferrin receptor-binding domain of any one of claims 52-56.

68. The antibody of claim 67, wherein the antibody is humanized.

69. The antibody of claim 67 or 68, further comprising a fusion partner.

70. The antibody of claim 69, wherein the fusion partner is selected from the group consisting of: an enzyme, a hormone, a growth factor, a cytokine, a chemokine, a glycolipid, a lipid, a soluble protein, a nucleic acid, and an additional antigen-binding domain.

71. The antibody of claim 69 or 70, wherein the fusion partner further comprises a linker between the fusion partner and the transferrin receptor-binding domain.

72. The antibody of claim 69 or 70, wherein the fusion partner is directly adjacent to the transferrin receptor-binding domain.

73. An antibody fragment comprising the transferrin receptor-binding domain of any one of claims 52-56.

74. The antibody fragment of claim 73, wherein the antibody fragment comprises a VHH domain.

75. The antibody fragment of claim 73 or 74, wherein the antibody fragment is humanized.

76. The antibody fragment of any one of claims 73-75, further comprising a fusion partner.

77. The antibody fragment of claim 76, wherein the fusion partner is selected from the group consisting of: an enzyme, a hormone, a growth factor, a cytokine, a chemokine, a glycolipid, a lipid, a soluble protein, a nucleic acid, and an additional antigen-binding domain.

78. The antibody fragment of claim 76 or 77, wherein the fusion partner further comprises a linker between the fusion partner and the transferrin receptor-binding domain.

79. The antibody fragment of claim 76 or 77, wherein the fusion partner is directly adjacent to the transferrin receptor-binding domain.

80. A composition comprising (i) the transferrin receptor-binding domain of any one of claims 52-56, the polypeptide of any one of claims 57-66, the antibody of any one of claims 67-72, or the antibody fragment of any one of claims 73-79, and (ii) a pharmaceutically acceptable carrier.

81. A kit comprising a composition of claim 80.

82. A nucleic acid encoding the transferrin receptor-binding domain of any one of claims 52-56, the polypeptide of any one of claims 57-66, the antibody of any one of claims 67-72, or the antibody fragment of any one of claims 73-79.

83. An expression vector comprising the nucleic acid of claim 82.

84. A host cell comprising the nucleic acid of claim 82 or the expression vector of claim 82.

85. A method of producing the transferrin receptor-binding domain, the polypeptide, the antibody, or the antibody fragment comprising:
(a) culturing the host cell of claim 84 in a culture medium under conditions sufficient to allow for the production of the transferrin receptor-binding domain, the polypeptide, the antibody, or the antibody fragment; and
(b) harvesting the transferrin receptor-binding domain, the polypeptide, the antibody, or the antibody fragment from the host cell or the culture medium.

86. The method of claim 85, further comprising isolating the transferrin receptor-binding domain, the polypeptide, the antibody, or the antibody fragment.

87. The method of claim 86, further comprising formulating the isolated transferrin receptor-binding domain, polypeptide, antibody, or antibody fragment.


SEQUENCE LISTING (221 sequences)

SEQ ID NO: 1 - skipped

SEQ ID NO: 2 - protein, 17 aa, synthetic construct
  VARIANT 3: note = X can be T or S
  VARIANT 4: note = X can be W, S, G,F or Y
  VARIANT 5: note = X can be S or N
  VARIANT 6: note = X can be G or A
  VARIANT 7: note = X can be R, S or G
  VARIANT 8: note = X can be H or A
  VARIANT 10: note = X can be L or Y
AIXXXXXXTXYADSVKG

SEQ ID NO: 3 - protein, 16 aa, synthetic construct
  VARIANT 2: note = X can be L or R
  VARIANT 4..5: note = X can be V or A
  VARIANT 6: note = X can be G or A
  VARIANT 7: note = X can be I or A
  VARIANT 8: note = X can be G or A
  VARIANT 9: note = X can be I or A
  VARIANT 10: note = X can be E or A
  VARIANT 11: note = X can be V or A
  VARIANT 12: note = X can be Q or A
  VARIANT 13: note = X can be T or A
  VARIANT 14: note = X can be Y or F
AXDXXXXXXXXXXXDY

SEQ ID NO: 4 - protein, 10 aa, synthetic construct
GLTSDTGGMG

SEQ ID NO: 5 - protein, 10 aa, synthetic construct
GFTSDTGGMG

SEQ ID NO: 6 - protein, 10 aa, synthetic construct
GLTFDTGGMG

SEQ ID NO: 7 - protein, 10 aa, synthetic construct
GLTSSTGGMG

SEQ ID NO: 8 - protein, 10 aa, synthetic construct
GLTSETGGMG

SEQ ID NO: 9 - protein, 10 aa, synthetic construct
GLTSDTYGMG

SEQ ID NO: 10 - protein, 10 aa, synthetic construct
GLTSDTGAMG

SEQ ID NO: 11 - protein, 10 aa, synthetic construct
GLTSDTGGMS

SEQ ID NO: 12 - protein, 10 aa, synthetic construct
GFTFSTGGMG

SEQ ID NO: 13 - protein, 10 aa, synthetic construct
GFTFSTGGMS

SEQ ID NO: 14 - protein, 10 aa, synthetic construct
GFTFSSGGMS

SEQ ID NO: 15 - protein, 17 aa, synthetic construct
AITWSGRHTLYADSVKG

SEQ ID NO: 16 - protein, 17 aa, synthetic construct
AISWSGRHTLYADSVKG

SEQ ID NO: 17 - protein, 17 aa, synthetic construct
AITSSGRHTLYADSVKG

SEQ ID NO: 18 - protein, 17 aa, synthetic construct
AITGSGRHTLYADSVKG

SEQ ID NO: 19 - protein, 17 aa, synthetic construct
AITWNGRHTLYADSVKG

SEQ ID NO: 20 - protein, 17 aa, synthetic construct
AITWSGSHTLYADSVKG

SEQ ID NO: 21 - protein, 17 aa, synthetic construct
AITWSGGHTLYADSVKG

SEQ ID NO: 22 - protein, 17 aa, synthetic construct
AITWSGRHTYYADSVKG

SEQ ID NO: 23 - protein, 17 aa, synthetic construct
AITWSARHTLYADSVKG

SEQ ID NO: 24 - protein, 17 aa, synthetic construct
AITWSGRATLYADSVKG

SEQ ID NO: 25 - protein, 17 aa, synthetic construct
AITFSGRHTLYADSVKG

SEQ ID NO: 26 - protein, 17 aa, synthetic construct
AITYSGRHTLYADSVKG

SEQ ID NO: 27 - protein, 16 aa, synthetic construct
ALDVVGIGIEVQTYDY

SEQ ID NO: 28 - protein, 16 aa, synthetic construct
ARDVVGIGIEVQTYDY

SEQ ID NO: 29 - protein, 16 aa, synthetic construct
ALDAVGIGIEVQTYDY

SEQ ID NO: 30 - protein, 16 aa, synthetic construct
ALDVAGIGIEVQTYDY

SEQ ID NO: 31 - protein, 16 aa, synthetic construct
ALDVVAIGIEVQTYDY

SEQ ID NO: 32 - protein, 16 aa, synthetic construct
ALDVVGAGIEVQTYDY

SEQ ID NO: 33 - protein, 16 aa, synthetic construct
ALDVVGIAIEVQTYDY

SEQ ID NO: 34 - protein, 16 aa, synthetic construct
ALDVVGIGAEVQTYDY

SEQ ID NO: 35 - protein, 16 aa, synthetic construct
ALDVVGIGIAVQTYDY

SEQ ID NO: 36 - protein, 16 aa, synthetic construct
ALDVVGIGIEAQTYDY

SEQ ID NO: 37 - protein, 16 aa, synthetic construct
ALDVVGIGIEVATYDY

SEQ ID NO: 38 - protein, 16 aa, synthetic construct
ALDVVGIGIEVQAYDY

SEQ ID NO: 39 - protein, 16 aa, synthetic construct
ALDVVGIGIEVQTFDY

SEQ ID NO: 40 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 41 - protein, 123 aa, synthetic construct
EVQLVESGGGLVQPGGSLRLSCAASGLTSDTGGMGWFRQAPGKGLEWVSAITWSGRHTLYADSVKGRFTISRDNSKNTLYLQMNSLRAEDTAVYYCALDVVGIGIEVQTYDYWGQGTLVTVSS

SEQ ID NO: 42 - protein, 123 aa, synthetic construct
EVQLVESGGGLVQPGGSLRLSCAASGLTSDTGGMGWFRQAPGKELEWVSAITWSGRHTLYADSVKGRFTISRDNSKNTLYLQMNSLRAEDTAVYYCALDVVGIGIEVQTYDYWGQGTLVTVSS

SEQ ID NO: 43 - protein, 123 aa, synthetic construct
EVQLVESGGGLVQPGGSLRLSCAASGLTSDTGGMGWFRQAPGKGREWVSAITWSGRHTLYADSVKGRFTISRDNSKNTLYLQMNSLRAEDTAVYYCALDVVGIGIEVQTYDYWGQGTLVTVSS

SEQ ID NO: 44 - protein, 123 aa, synthetic construct
EVQLVESGGGLVQPGGSLRLSCAASGLTSDTGGMGWFRQAPGKEREWVSAITWSGRHTLYADSVKGRFTISRDNSKNTLYLQMNSLRAEDTAVYYCALDVVGIGIEVQTYDYWGQGTLVTVSS

SEQ ID NO: 45 - protein, 123 aa, synthetic construct
EVQLVESGGGLVQPGGSLRLSCAASGLTSDTGGMGWFRQAPGKEREFVSAITWSGRHTLYADSVKGRFTISRDNSKNTVYLQMNSLRAEDTAVYYCALDVVGIGIEVQTYDYWGQGTLVTVSS

SEQ ID NO: 46 - protein, 123 aa, synthetic construct
EVQLVESGGGLVQPGGSLRLSCAASGLTSDTGGMGWFRQAPGKEREFVSAITWSGRHTLYADSVKGRFTISRDKSKNTVYLQMNSLRAEDTAVYYCALDVVGIGIEVQTYDYWGQGTLVTVSS

SEQ ID NO: 47 - protein, 123 aa, synthetic construct
EVQLVESGGGLVQPGGSLRLSCAASGLTSDTGGMGWFRQAPGKGLEWVSAITWSGRHTLYADSVKGRFTISRDKSKNTVYLQMNSLRAEDTAVYYCALDVVGIGIEVQTYDYWGQGTLVTVSS

SEQ ID NO: 48 - protein, 123 aa, synthetic construct
EVQLVESGGGLVQPGGSLRLSCAASGLTSDTGGMGWFRQAPGKELEWVSAITWSGRHTLYADSVKGRFTISRDNSKNTVYLQMNSLRAEDTAVYYCALDVVGIGIEVQTYDYWGQGTLVTVSS

SEQ ID NO: 49 - protein, 123 aa, synthetic construct
EVQLVESGGGLVQPGGSLRLSCAASGLTSDTGGMGWFRQAPGKGREWVSAITWSGRHTLYADSVKGRFTISRDNSKNTVYLQMNSLRAEDTAVYYCALDVVGIGIEVQTYDYWGQGTLVTVSS

SEQ ID NO: 50 - protein, 123 aa, synthetic construct
EVQLVESGGGLVQPGGSLRLSCAASGLTSDTGGMGWFRQAPGKEREWVSAITWSGRHTLYADSVKGRFTISRDNSKNTVYLQMNSLRAEDTAVYYCALDVVGIGIEVQTYDYWGQGTLVTVSS

SEQ ID NO: 51 - protein, 123 aa, synthetic construct
EVQLVESGGGLVQPGGSLRLSCAASGLTSDTGGMGWFRQAPGKGLEWVSAITWSGRHTLYADSVKGRFTISRDNSKNTVYLQMNSLRAEDTAVYYCALDVVGIGIEVQTYDYWGQGTLVTVSS

SEQ ID NO: 52 - protein, 123 aa, synthetic construct
EVQLVESGGGLVQPGGSLRLSCAASGLTSDTGGMGWFRQAPGKELEWVSAITWSGRHTLYADSVKGRFTISRDKSKNTVYLQMNSLRAEDTAVYYCALDVVGIGIEVQTYDYWGQGTLVTVSS

SEQ ID NO: 53 - protein, 123 aa, synthetic construct
EVQLVESGGGLVQPGGSLRLSCAASGLTSDTGGMGWFRQAPGKGREWVSAITWSGRHTLYADSVKGRFTISRDKSKNTVYLQMNSLRAEDTAVYYCALDVVGIGIEVQTYDYWGQGTLVTVSS

SEQ ID NO: 54 - protein, 123 aa, synthetic construct
EVQLVESGGGLVQPGGSLRLSCAASGLTSDTGGMGWFRQAPGKEREWVSAITWSGRHTLYADSVKGRFTISRDKSKNTVYLQMNSLRAEDTAVYYCALDVVGIGIEVQTYDYWGQGTLVTVSS

SEQ ID NO: 55 - protein, 123 aa, synthetic construct
QVQLVESGGGLVQTGGSLRLSCTASGFTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 56 - protein, 123 aa, synthetic construct
QVQLVESGGGLVQTGGSLRLSCTASGLTFDTGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 57 - protein, 123 aa, synthetic construct
QVQLVESGGGLVQTGGSLRLSCTASGLTSSTGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 58 - protein, 123 aa, synthetic construct
QVQLVESGGGLVQTGGSLRLSCTASGLTSETGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 59 - protein, 123 aa, synthetic construct
QVQLVESGGGLVQTGGSLRLSCTASGLTSDTYGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 60 - protein, 123 aa, synthetic construct
QVQLVESGGGLVQTGGSLRLSCTASGLTSDTGAMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 61 - protein, 123 aa, synthetic construct
QVQLVESGGGLVQTGGSLRLSCTASGLTSDTGGMSWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 62 - protein, 123 aa, synthetic construct
QVQLVESGGGLVQTGGSLRLSCTASGLTSDTGGMGWFRQAAGKERDFVSAISWSGRHTLYADSVKGRFNISRDKGKNTVFLQMNSLKPEDTAVYYCALDVVGIGIEVQTYDYWGQGTQVTVSS

SEQ ID NO: 63 - protein, 123 aa, synthetic construct
QVQLVESGGGLVQTGGSLRLSCTASGLTSDTGGMGWFRQAAGKERDFVSAITSSGRHTLYADSVKGRFNISRDKGKNTVFLQMNSLKPEDTAVYYCALDVVGIGIEVQTYDYWGQGTQVTVSS

SEQ ID NO: 64 - protein, 123 aa, synthetic construct
QVQLVESGGGLVQTGGSLRLSCTASGLTSDTGGMGWFRQAAGKERDFVSAITGSGRHTLYADSVKGRFNISRDKGKNTVFLQMNSLKPEDTAVYYCALDVVGIGIEVQTYDYWGQGTQVTVSS

SEQ ID NO: 65 - protein, 123 aa, synthetic construct
QVQLVESGGGLVQTGGSLRLSCTASGLTSDTGGMGWFRQAAGKERDFVSAITWNGRHTLYADSVKGRFNISRDKGKNTVFLQMNSLKPEDTAVYYCALDVVGIGIEVQTYDYWGQGTQVTVSS

SEQ ID NO: 66 - protein, 123 aa, synthetic construct
QVQLVESGGGLVQTGGSLRLSCTASGLTSDTGGMGWFRQAAGKERDFVSAITWSGSHTLYADSVKGRFNISRDKGKNTVFLQMNSLKPEDTAVYYCALDVVGIGIEVQTYDYWGQGTQVTVSS

SEQ ID NO: 67 - protein, 123 aa, synthetic construct
QVQLVESGGGLVQTGGSLRLSCTASGLTSDTGGMGWFRQAAGKERDFVSAITWSGGHTLYADSVKGRFNISRDKGKNTVFLQMNSLKPEDTAVYYCALDVVGIGIEVQTYDYWGQGTQVTVSS

SEQ ID NO: 68 - protein, 123 aa, synthetic construct
QVQLVESGGGLVQTGGSLRLSCTASGLTSDTGGMGWFRQAAGKERDFVSAITWSGRHTYYADSVKGRFNISRDKGKNTVFLQMNSLKPEDTAVYYCALDVVGIGIEVQTYDYWGQGTQVTVSS

SEQ ID NO: 69 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCARDVVGIGIEVQTYDYWGQGTQVTVSS

SEQ ID NO: 70 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDAVGIGIEVQTYDYWGQGTQVTVSS

SEQ ID NO: 71 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVAGIGIEVQTYDYWGQGTQVTVSS

SEQ ID NO: 72 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVAIGIEVQTYDYWGQGTQVTVSS

SEQ ID NO: 73 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGAGIEVQTYDYWGQGTQVTVSS

SEQ ID NO: 74 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIAIEVQTYDYWGQGTQVTVSS

SEQ ID NO: 75 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGAEVQTYDYWGQGTQVTVSS

SEQ ID NO: 76 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIAVQTYDYWGQGTQVTVSS

SEQ ID NO: 77 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEAQTYDYWGQGTQVTVSS

SEQ ID NO: 78 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVATYDYWGQGTQVTVSS

SEQ ID NO: 79 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQAYDYWGQGTQVTVSS

SEQ ID NO: 80 - protein, 123 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTFDYWGQGTQVTVSS

SEQ ID NO: 81 - protein, 123 aa, synthetic construct
QVQLVESGGGLVQTGGSLRLSCTASGLTSDTGGMGWFRQAAGKERDFVSAITWSARHTLYADSVKGRFNISRDKGKNTVFLQMNSLKPEDTAVYYCALDVVGIGIEVQTYDYWGQGTQVTVSS

SEQ ID NO: 82 - protein, 123 aa, synthetic construct
QVQLVESGGGLVQTGGSLRLSCTASGLTSDTGGMGWFRQAAGKERDFVSAITWSGRATLYADSVKGRFNISRDKGKNTVFLQMNSLKPEDTAVYYCALDVVGIGIEVQTYDYWGQGTQVTVSS

SEQ ID NO: 83 - protein, 123 aa, synthetic construct
QVQLVESGGGLVQTGGSLRLSCTASGLTSDTGGMGWFRQAAGKERDFVSAITFSGRHTLYADSVKGRFNISRDKGKNTVFLQMNSLKPEDTAVYYCALDVVGIGIEVQTYDYWGQGTQVTVSS

SEQ ID NO: 84 - protein, 123 aa, synthetic construct
EVQLVESGGGLVQPGGSLRLSCAASGFTFSTGGMGWFRQAPGKEREWVSAITWSGRHTLYADSVKGRFTISRDKSKNTVYLQMNSLRAEDTAVYYCALDVVGIGIEVQTYDYWGQGTLVTVSS

SEQ ID NO: 85 - protein, 123 aa, synthetic construct
EVQLVESGGGLVQPGGSLRLSCAASGFTFSTGGMSWFRQAPGKEREWVSAITWSGRHTLYADSVKGRFTISRDKSKNTVYLQMNSLRAEDTAVYYCALDVVGIGIEVQTYDYWGQGTLVTVSS

SEQ ID NO: 86 - protein, 123 aa, synthetic construct
EVQLVESGGGLVQPGGSLRLSCAASGFTFSSGGMSWFRQAPGKEREWVSAITWSGRHTLYADSVKGRFTISRDKSKNTVYLQMNSLRAEDTAVYYCALDVVGIGIEVQTYDYWGQGTLVTVSS

SEQ ID NO: 87 - protein, 123 aa, synthetic construct
EVQLVESGGGLVQPGGSLRLSCAASGFTFSTGGMSWFRQAPGKEREWVSAITFSGRHTLYADSVKGRFTISRDKSKNTVYLQMNSLRAEDTAVYYCALDVVGIGIEVQTYDYWGQGTLVTVSS

SEQ ID NO: 88 - protein, 123 aa, synthetic construct
EVQLVESGGGLVQPGGSLRLSCAASGFTFSTGGMSWFRQAPGKEREWVSAITYSGRHTLYADSVKGRFTISRDKSKNTVYLQMNSLRAEDTAVYYCALDVVGIGIEVQTYDYWGQGTLVTVSS

SEQ ID NO: 89 - protein, 357 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 361 aa, synthetic construct
EVQLVESGGGLVQPGGSLRLSCAASGLTSDTGGMGWFRQAPGKGLEWVSAITWSGRHTLYADSVKGRFTISRDNSKNTLYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 361 aa, synthetic construct
EVQLVESGGGLVQPGGSLRLSCAASGLTSDTGGMGWFRQAPGKELEWVSAITWSGRHTLYADSVKGRFTISRDNSKNTLYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 361 aa, synthetic construct
EVQLVESGGGLVQPGGSLRLSCAASGLTSDTGGMGWFRQAPGKGREWVSAITWSGRHTLYADSVKGRFTISRDNSKNTLYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 361 aa, synthetic construct
EVQLVESGGGLVQPGGSLRLSCAASGLTSDTGGMGWFRQAPGKEREWVSAITWSGRHTLYADSVKGRFTISRDNSKNTLYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 361 aa, synthetic construct
EVQLVESGGGLVQPGGSLRLSCAASGLTSDTGGMGWFRQAPGKEREFVSAITWSGRHTLYADSVKGRFTISRDNSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 361 aa, synthetic construct
EVQLVESGGGLVQPGGSLRLSCAASGLTSDTGGMGWFRQAPGKEREFVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 361 aa, synthetic construct
EVQLVESGGGLVQPGGSLRLSCAASGLTSDTGGMGWFRQAPGKGLEWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 361 aa, synthetic construct
EVQLVESGGGLVQPGGSLRLSCAASGLTSDTGGMGWFRQAPGKELEWVSAITWSGRHTLYADSVKGRFTISRDNSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 361 aa, synthetic construct
EVQLVESGGGLVQPGGSLRLSCAASGLTSDTGGMGWFRQAPGKGREWVSAITWSGRHTLYADSVKGRFTISRDNSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 361 aa, synthetic construct
EVQLVESGGGLVQPGGSLRLSCAASGLTSDTGGMGWFRQAPGKEREWVSAITWSGRHTLYADSVKGRFTISRDNSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 361 aa, synthetic construct
EVQLVESGGGLVQPGGSLRLSCAASGLTSDTGGMGWFRQAPGKGLEWVSAITWSGRHTLYADSVKGRFTISRDNSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 361 aa, synthetic construct
EVQLVESGGGLVQPGGSLRLSCAASGLTSDTGGMGWFRQAPGKELEWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 361 aa, synthetic construct
EVQLVESGGGLVQPGGSLRLSCAASGLTSDTGGMGWFRQAPGKGREWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 361 aa, synthetic construct
EVQLVESGGGLVQPGGSLRLSCAASGLTSDTGGMGWFRQAPGKEREWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 361 aa, synthetic construct
QVQLVESGGGLVQTGGSLRLSCTASGFTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 361 aa, synthetic construct
QVQLVESGGGLVQTGGSLRLSCTASGLTFDTGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 361 aa, synthetic construct
QVQLVESGGGLVQTGGSLRLSCTASGLTSSTGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 361 aa, synthetic construct
QVQLVESGGGLVQTGGSLRLSCTASGLTSETGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 361 aa, synthetic construct
QVQLVESGGGLVQTGGSLRLSCTASGLTSDTYGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 361 aa, synthetic construct
QVQLVESGGGLVQTGGSLRLSCTASGLTSDTGAMGWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 361 aa, synthetic construct
QVQLVESGGGLVQTGGSLRLSCTASGLTSDTGGMSWFRQAAGKERDFVSAITWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 361 aa, synthetic construct
QVQLVESGGGLVQTGGSLRLSCTASGLTSDTGGMGWFRQAAGKERDFVSAISWSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 361 aa, synthetic construct
QVQLVESGGGLVQTGGSLRLSCTASGLTSDTGGMGWFRQAAGKERDFVSAITSSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 361 aa, synthetic construct
QVQLVESGGGLVQTGGSLRLSCTASGLTSDTGGMGWFRQAAGKERDFVSAITGSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 361 aa, synthetic construct
QVQLVESGGGLVQTGGSLRLSCTASGLTSDTGGMGWFRQAAGKERDFVSAITWNGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 361 aa, synthetic construct
QVQLVESGGGLVQTGGSLRLSCTASGLTSDTGGMGWFRQAAGKERDFVSAITWSGSHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 361 aa, synthetic construct
QVQLVESGGGLVQTGGSLRLSCTASGLTSDTGGMGWFRQAAGKERDFVSAITWSGGHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 361 aa, synthetic construct
QVQLVESGGGLVQTGGSLRLSCTASGLTSDTGGMGWFRQAAGKERDFVSAITWSGRHTYYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCARDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDAVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVAGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVAIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGAGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIAIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGAEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIAVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEAQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVATYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQAYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 361 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTFDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 361 aa, synthetic construct
QVQLVESGGGLVQTGGSLRLSCTASGLTSDTGGMGWFRQAAGKERDFVSAITWSARHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 361 aa, synthetic construct
QVQLVESGGGLVQTGGSLRLSCTASGLTSDTGGMGWFRQAAGKERDFVSAITWSGRATLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 361 aa, synthetic construct
QVQLVESGGGLVQTGGSLRLSCTASGLTSDTGGMGWFRQAAGKERDFVSAITFSGRHTLYADSVKGRFNISRDKGKNTVFLQMNSLKPEDTAVYYCALDVVGIGIEVQTYDYWGQGTQVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 361 aa, synthetic construct
EVQLVESGGGLVQPGGSLRLSCAASGFTFSTGGMGWFRQAPGKEREWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 361 aa, synthetic construct
EVQLVESGGGLVQPGGSLRLSCAASGFTFSTGGMSWFRQAPGKEREWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 361 aa, synthetic construct
EVQLVESGGGLVQPGGSLRLSCAASGFTFSSGGMSWFRQAPGKEREWVSAITWSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 361 aa, synthetic construct
EVQLVESGGGLVQPGGSLRLSCAASGFTFSTGGMSWFRQAPGKEREWVSAITFSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 361 aa, synthetic construct
EVQLVESGGGLVQPGGSLRLSCAASGFTFSTGGMSWFRQAPGKEREWVSAITYSGRHTLYADSVKGRFTISRDKSKNTVYLQMNSLRAEDTAVYYCALDVVGIGIEVQTYDYWGQGTLVTVSSGGGGASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 625 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGSARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 140 - protein, 630 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSGGGGSGGGGSARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 141 - protein, 625 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGGGGSQVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 142 - protein, 630 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGGGGSGGGGSQVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSS

SEQ ID NO: 143 - protein, 630 aa, synthetic construct
EVQLVESGGGLVQPGGSLRLSCAASGFTFSTGGMGWFRQAPGKEREWVSAITWSGRHTLYADSVKGRFTISRDKSKNTVYLQMNSLRAEDTAVYYCALDVVGIGIEVQTYDYWGQGTLVTVSSGGGGSGGGGSARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 144 - protein, 630 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQGGGGSGGGGSEVQLVESGGGLVQPGGSLRLSCAASGFTFSTGGMGWFRQAPGKEREWVSAITWSGRHTLYADSVKGRFTISRDKSKNTVYLQMNSLRAEDTAVYYCALDVVGIGIEVQTYDYWGQGTLVTVSS

SEQ ID NO: 145 - protein, 714 aa, synthetic construct
QVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSEPKSSGGGGSGGGGSTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 146 - protein, 727 aa, synthetic construct
HHHHHHENLYFQGQVQLVESGGGLVQTGGSLRLSCTASGLTSDTGGMGWFRQAAGKERDFVSAITWSGRHTLYADSVKGRFNISRDKGKNTVFLQMNSLKPEDTAVYYCALDVVGIGIEVQTYDYWGQGTQVTVSSEPKSSGGGGSGGGGSTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 147 - protein, 123 aa, synthetic construct
QVQLVESGGGLVQTGGSLRLSCTASGLTSDTGGMGWFRQAAGKERDFVSAITWSGRHTLYADSVKGRFTISRDKGKNTVFLQMNSLKPEDTAVYYCALDVVGIGIEVQTYDYWGQGTQVTVSS

SEQ ID NO: 148 - protein, 714 aa, synthetic construct
QVQLVESGGGLVQTGGSLRLSCTASGLTSDTGGMGWFRQAAGKERDFVSAITWSGRHTLYADSVKGRFTISRDKGKNTVFLQMNSLKPEDTAVYYCALDVVGIGIEVQTYDYWGQGTQVTVSSEPKSSGGGGSGGGGSTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 149 - protein, 760 aa, synthetic construct
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 150 - protein, 679 aa, synthetic construct
MIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 151 - protein, 478 aa, synthetic construct
MDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 152 - protein, 760 aa, synthetic construct; identical to SEQ ID NO: 149

SEQ ID NO: 153 - protein, 5 aa, synthetic construct
  REGION 1..5: note = The entire sequence of amino acids 1-5 can be repeated 1 to 10 times
GGGGS

SEQ ID NO: 154 - protein, 5 aa, synthetic construct
GGGGS

SEQ ID NO: 155 - protein, 10 aa, synthetic construct
GGGGSGGGGS

SEQ ID NO: 156 - protein, 15 aa, synthetic construct
GGGGSGGGGSGGGGS

SEQ ID NO: 157 - protein, 20 aa, synthetic construct
GGGGSGGGGSGGGGSGGGGS

SEQ ID NO: 158 - protein, 18 aa, synthetic construct
KESGSVSSEQLAQFRSLD

SEQ ID NO: 159 - protein, 14 aa, synthetic construct
EGKSSGSGSESKST

SEQ ID NO: 160 - protein, 8 aa, synthetic construct
GGGGGGGG

SEQ ID NO: 161 - protein, 6 aa, synthetic construct
GGGGGG

SEQ ID NO: 162 - protein, 12 aa, synthetic construct
GSAGSAAGSGEF

SEQ ID NO: 163 - protein, 5 aa, synthetic construct
  REGION 1..5: note = The entire sequence of amino acids 1-5 can be repeated 1 to 10 times
EAAAK

SEQ ID NO: 164 - protein, 6 aa, synthetic construct
  REGION 2..6: note = The amino acids 2-6 can be repeated 2 to 5 times
AEAAAK

SEQ ID NO: 165 - protein, 14 aa, synthetic construct
APAPAPAPAPAPAP

SEQ ID NO: 166 - DNA, 251 bp, synthetic construct
  source 1..251: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 167 - DNA, 418 bp, synthetic construct
  source 1..418: mol_type = other DNA
ggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 168 - DNA, 146 bp, synthetic construct
  source 1..146: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatcctc

SEQ ID NO: 169 - DNA, 202 bp, synthetic construct
  source 1..202: mol_type = other DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtc

SEQ ID NO: 170 - DNA, 366 bp, synthetic construct
  source 1..366: mol_type = other DNA
gggggaggctgctggtgaatattaaccaaggtcaccccagttatcggaggagcaaacaggggctaagtccacgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcaagaggtaagggtttaagggatggttggttggtggggnattaatgtttaattacctggagcacctgcctgaaatcactttttttcaggttgg

SEQ ID NO: 171 - DNA, 559 bp, synthetic construct
  source 1..559: mol_type = other DNA
agatctctgggcctcaaaatggagatggatcccaggtcttgtgggaccctgggatgtttggggactttctatctagcaccccagtaggcctgtcctggccagagaagactggtaggggccgagtggggtttgaaggcagccggcccggcccagcccaggagcgctatttattgcatatttattgtttggatgtcaccatcagagacgaagggaagggtagccagggagggagtccagcccagctgcctgcaggagaatctggctcagtctactatgggcagggccccccaccaagctgagccgaatggagacagctgagctgaggcctgactttttcaataaaacattgtgtagttctgggcctcctgctgccccggctctgtttcccctggcgccaagagaagaaggcggaactgaacccaggcccagagccggctccctgaggctgtgcccctttccggcaatctctggccacaacccccactggccaggccgtccctcccactggccctagggcccctcccactcccacaccagataaggacagcccagtgccgtc

SEQ ID NO: 172 - DNA, 404 bp, synthetic construct
  source 1..404: mol_type = other DNA
agatctgagagtaggtgtttgtccaaagtttatatgccaaggctgtgagtgaaacaggagcttcgatcttttggtgttccatctacaacatacacaaaacaaaagatggagaatgagaagtccaggcaaccccggaaacaacaagtttctgtcaaaagcaataatgaactgtttgtgccattaacaaaaacgttatgaagacagaaaccatctcccaaagatttcataacagagccacataagtggaaagtaaatgattaaagaatgtgggtctcagagttccattcaaatcatgatactttatcttctatttacaaagataaaagtacaccagaaaatggttaatgtttaagcgctttcatatttggctctgtctttttagcagacgaaaaccactttggcag

SEQ ID NO: 173 - DNA, 92 bp, synthetic construct
  source 1..92: mol_type = other DNA
aagaggtaagggtttaagggatggttggttggtggggnattaatgtttaattacctggagcacctgcctgaaatcactttttttcaggttgg

SEQ ID NO: 174 - DNA, 328 bp, synthetic construct
  source 1..328: mol_type = other DNA
caggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggg

SEQ ID NO: 175 - DNA, 72 bp, synthetic construct
  source 1..72: mol_type = other DNA
gggggaggctgctggtgaatattaaccaaggtcaccccagttatcggaggagcaaacaggggctaagtccac

SEQ ID NO: 176 - protein, 39 aa, synthetic construct
MEFSSPSREECPKPLSRVSIMAGSLTGLLLLQAVSWASG

SEQ ID NO: 177 - protein, 19 aa, synthetic construct
MGWSCIILFLVATATGAYA

SEQ ID NO: 178 - DNA, 1491 bp, synthetic construct
  source 1..1491: mol_type = other DNA
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccag

SEQ ID NO: 179 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 180 - DNA, 1731 bp, synthetic construct
  source 1..1731: mol_type = other DNA
acgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 181 - protein, 576 aa, synthetic construct
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 182 - protein, 13 aa, synthetic construct
HHHHHHENLYFQG

SEQ ID NO: 183 - protein, 10 aa, synthetic construct
  VARIANT 2: note = X can be S or F
  VARIANT 6: note = X can be D, H, or Y
  VARIANT 7: note = X can be Y or F
  VARIANT 10: note = X can be G or A
GXTLDXXAIX

SEQ ID NO: 184 - protein, 17 aa, synthetic construct
  VARIANT 4: note = X can be S or R
  VARIANT 5: note = X can be S or G
  VARIANT 8: note = X can be I or R
  VARIANT 10: note = X can be F or Y
  VARIANT 12: note = X can be A or G
  VARIANT 14: note = X can be F or S
CISXXDGXTXYXDXVKG

SEQ ID NO: 185 - protein, 21 aa, synthetic construct
  VARIANT 2: note = X can be A or S
  VARIANT 3: note = X can be K or H
  VARIANT 4: note = X can be Y or D
  VARIANT 8: note = X can be I or V
  VARIANT 15: note = X can be V or E
  VARIANT 17: note = X can be P or Q
  VARIANT 18: note = X can be V, I, or L
AXXXGPNXCRGWLWXPXXSGS

SEQ ID NO: 186 - protein, 10 aa, synthetic construct
GSTLDDYAIG

SEQ ID NO: 187 - protein, 10 aa, synthetic construct
GSTLDHYAIG

SEQ ID NO: 188 - protein, 10 aa, synthetic construct
GFTLDYFAIA

SEQ ID NO: 189 - protein, 17 aa, synthetic construct
CISSSDGITFYGDFVKG

SEQ ID NO: 190 - protein, 17 aa, synthetic construct
CISRSDGITYYADSVKG

SEQ ID NO: 191 - protein, 17 aa, synthetic construct
CISSGDGRTFYADSVKG

SEQ ID NO: 192 - protein, 21 aa, synthetic construct
AAKYGPNICRGWLWVPPVSGS

SEQ ID NO: 193 - protein, 21 aa, synthetic construct
AAHYGPNVCRGWLWEPPISGS

SEQ ID NO: 194 - protein, 21 aa, synthetic construct
ASHDGPNVCRGWLWVPQLSGS

SEQ ID NO: 195 - skipped

SEQ ID NO: 196 - protein, 17 aa, synthetic construct
  VARIANT 3: note = X can be T or S
  VARIANT 4: note = X can be W, S, G, F, or Y
  VARIANT 5: note = X can be S or N
  VARIANT 6: note = X can be G or A
  VARIANT 7: note = X can be R, S, or G
  VARIANT 8: note = X can be H or A
  VARIANT 10: note = X can be L or Y
AIXXXXXXTXYADSVKG

SEQ ID NO: 197 - protein, 16 aa, synthetic construct
  VARIANT 2: note = X can be L or R
  VARIANT 4..5: note = X can be V or A
  VARIANT 6: note = X can be G or A
  VARIANT 7: note = X can be I or A
  VARIANT 8: note = X can be G or A
  VARIANT 9: note = X can be I or A
  VARIANT 10: note = X can be E or A
  VARIANT 11: note = X can be V or A
  VARIANT 12: note = X can be Q or A
  VARIANT 13: note = X can be T or A
  VARIANT 14: note = X can be Y or F
AXDXXXXXXXXXXXDY

SEQ ID NO: 198 - protein, 10 aa, synthetic construct
GLTSDTGGMG

SEQ ID NO: 199 - protein, 17 aa, synthetic construct
AITWSGRHTLYADSVKG

SEQ ID NO: 200 - protein, 16 aa, synthetic construct
ALDVVGIGIEVQTYDY

SEQ ID NO: 201 - protein, 10 aa, synthetic construct
ESAFSLNAIG

SEQ ID NO: 202 - protein, 17 aa, synthetic construct
GIGTDGITTYYADFVKD

SEQ ID NO: 203 - protein, 17 aa, synthetic construct
NAGSWRTVLSGTHVSRS

SEQ ID NO: 204 - protein, 10 aa, synthetic construct
GRDYNHFQRA

SEQ ID NO: 205 - protein, 16 aa, synthetic construct
RITWSGTITYNESVKG

SEQ ID NO: 206 - protein, 17 aa, synthetic construct
ALKTQPPLSQDAGDYTY

SEQ ID NO: 207 - protein, 10 aa, synthetic construct
GRSLSTYVMG

SEQ ID NO: 208 - protein, 15 aa, synthetic construct
ARNGMSTYYTDSVKD

SEQ ID NO: 209 - protein, 16 aa, synthetic construct
AGDRSWSRLLRGEYEY

SEQ ID NO: 210 - protein, 121 aa, synthetic construct
QLQLVESGGGLVQPGGSLRLSCAGSGRSLSTYVMGWFRQAPGKERELVAARNGMSTYYTDSVKDRSAISRDNAKNTVYLEMNSLEPEDTAVYYCAGDRSWSRLLRGEYEYWGQGTQVTVSS

SEQ ID NO: 211 - protein, 124 aa, synthetic construct
QVQLVESGGGLVQAGGSLRLSCAASESAFSLNAIGWYRQAPGNQRELVAGIGTDGITTYYADFVKDRFTISRDNVKNTVYLQMNSLKPEDTAVYYCNAGSWRTVLSGTHVSRSWGPGTQVTVSS

SEQ ID NO: 212 - protein, 123 aa, synthetic construct
QLQLVESGGGLVKTGGSLRLSCTDSGRDYNHFQRAWFRQAPGKEREFVARITWSGTITYNESVKGRFTISGDDASNTIHLQMNSLKSDDTAIYYCALKTQPPLSQDAGDYTYWGQGTQVTVSS

SEQ ID NO: 213 - protein, 128 aa, synthetic construct
QVQLVESGGGLVQPGGSLRLSCTTSGSTLDDYAIGWFRQAPGKEREGVSCISSSDGITFYGDFVKGRFTISRDNAKNSVYLQMNNLKPEDTAVYYCAAKYGPNICRGWLWVPPVSGSWGQGTQVTVSS

SEQ ID NO: 214 - protein, 128 aa, synthetic construct
QVQLVESGGGVVQPGGSLRLSCAASGSTLDHYAIGWFRQAPGKEREGVSCISRSDGITYYADSVKGRFTISRDNAKNTVYLQMNSLKPEDTAVYYCAAHYGPNVCRGWLWEPPISGSWGQGTQVTVSS

SEQ ID NO: 215 - protein, 128 aa, synthetic construct
QLQLVESGGGLVQPGESLRLSCVPSGFTLDYFAIAWFRQAPGKEREGVSCISSGDGRTFYADSVKGRFTISRDNSKNTVYLQMNSLKPEDTAVYYCASHDGPNVCRGWLWVPQLSGSWGQGTQVTVSS

SEQ ID NO: 216 - protein, 355 aa, synthetic construct
QLQLVESGGGLVQPGGSLRLSCAGSGRSLSTYVMGWFRQAPGKERELVAARNGMSTYYTDSVKDRSAISRDNAKNTVYLEMNSLEPEDTAVYYCAGDRSWSRLLRGEYEYWGQGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 358 aa, synthetic construct
QVQLVESGGGLVQAGGSLRLSCAASESAFSLNAIGWYRQAPGNQRELVAGIGTDGITTYYADFVKDRFTISRDNVKNTVYLQMNSLKPEDTAVYYCNAGSWRTVLSGTHVSRSWGPGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 357 aa, synthetic construct
QLQLVESGGGLVKTGGSLRLSCTDSGRDYNHFQRAWFRQAPGKEREFVARITWSGTITYNESVKGRFTISGDDASNTIHLQMNSLKSDDTAIYYCALKTQPPLSQDAGDYTYWGQGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 362 aa, synthetic construct
QVQLVESGGGLVQPGGSLRLSCTTSGSTLDDYAIGWFRQAPGKEREGVSCISSSDGITFYGDFVKGRFTISRDNAKNSVYLQMNNLKPEDTAVYYCAAKYGPNICRGWLWVPPVSGSWGQGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 362 aa, synthetic construct
QVQLVESGGGVVQPGGSLRLSCAASGSTLDHYAIGWFRQAPGKEREGVSCISRSDGITYYADSVKGRFTISRDNAKNTVYLQMNSLKPEDTAVYYCAAHYGPNVCRGWLWEPPISGSWGQGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 362 aa, synthetic construct
QLQLVESGGGLVQPGESLRLSCVPSGFTLDYFAIAWFRQAPGKEREGVSCISSGDGRTFYADSVKGRFTISRDNSKNTVYLQMNSLKPEDTAVYYCASHDGPNVCRGWLWVPQLSGSWGQGTQVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK